(19) United States
(12) Patent Application Publication
(10) Pub. No.: US 2025/0262186 A1
(43) Pub. Date: Aug. 21, 2025

(54) NOVEL SUBSTITUTED FLUORINATED N-PROPYL-PYRROLIDINE AND N-PROPYL-AZETIDINE COMPOUNDS, PROCESSES FOR THEIR PREPARATION AND THERAPEUTIC USES THEREOF

(71) Applicant: SANOFI, Paris (FR)

(72) Inventors: Youssef EL-AHMAD, Paris (FR); Frank Halley, Paris (FR)

(21) Appl. No.: 18/856,717

(22) PCT Filed: Apr. 14, 2023
(86) PCT No.: PCT/EP2023/059811

§ 371 Date: Oct. 14, 2024

(30) Foreign Application Priority Data
Apr. 15, 2022 (EP) 22305567.4

(51) Int. Cl. A61K 31/4015; A61K 31/397; A61K 31/40; C07D 205/04; C07D 207/08; C07D 207/12; C07D 207/14

(52) U.S. Cl. CPC A61K 31/4015; A61K 31/397; A61K 31/40; C07D 205/04; C07D 207/08; C07D 207/12; C07D 207/14

(57) ABSTRACT

Disclosed herein are compounds of the formula (I), or pharmaceutically acceptable salts thereof: wherein R1 and R2 independently represent a hydrogen atom or a deuterium atom; R3 represents a hydrogen atom, a —COOH group or a —OH group; R3′ and R3″ independently represent a hydrogen atom, a methyl group, a methoxy group, a chlorine atom, a fluorine atom, or a cyano group; R4 represents a hydrogen atom or a fluorine atom; R5 and R5′ independently represent a hydrogen atom or a fluorine atom; R6 represents a phenyl group, a fused phenyl group, a bicyclic group comprising 5 to 12 carbon atoms, a heteroaryl group comprising 2 to 9 carbon atoms and comprising from 1 to 3 heteroatoms, a cycloalkyl group comprising 3 to 7 carbon atoms, a (C₃-C₆)cycloalkyl(C₁-C₃)alkyl group, a 4 to 7 membered-heterocycloalkyl group, a (C₁-C₆)alkyl group, or a phenyl(C₁-C₂)alkyl group; X represents —CH₂—, —O— or —S—; Y represents —CH₂—, —O— or —NH—; R7 independently represents a (C₁-C₃)alkyl group, such as a methyl group, a halogen atom, such as a fluorine atom, a cyano group, or a (C₁-C₃)fluoroalkyl group, such as a trifluoromethyl; R8 represents a hydrogen atom, or a (C₁-C₃)alkyl group or a cyclopropyl; same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

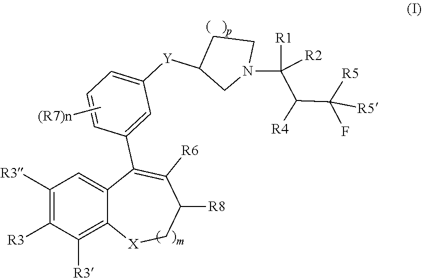

Disclosed herein are novel substituted fluorinated N-propyl-pyrrolidine and N-propyl-azetidine derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and differentiation in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1—blocking estrogen synthesis by aromatase inhibitors that are used to treat early and advanced ERα-positive breast cancer patients; 2—antagonizing estrogen ligand binding to ERα by tamoxifen which is used to treat ERα-positive breast cancer patients in both pre- and post-menopausal setting; 3—antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de-novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669, WO2018/091153 and the article of Youssef El-Ahmad et al. (J. Med. Chem., 2020, 63, 512-528) disclose some substituted 6,7-dihydro-5H-benzo[7]annulene compounds and substituted N-(3-fluoropropyl)-pyrrolidine derivatives useful as SERDs. Such compounds have O-pyrrolidine side chains in the para position and are (S) stereoisomers. However, the article of James S. Scott et al. (J. Med. Chem., 2023, 66, 2918-2945) which relates to SERD compounds with a tricyclic scaffold and a basic amine-containing lateral chain, shows that the change of the lateral chain from the para position to the meta position, has an impact on the biological profile of the compounds.

The inventors have now found novel compounds able to selectively antagonize and degrade the estrogen receptors (SERDs compounds), for use in cancer treatment.

Disclosed herein are compounds of the formula (I), or pharmaceutically acceptable salts thereof:

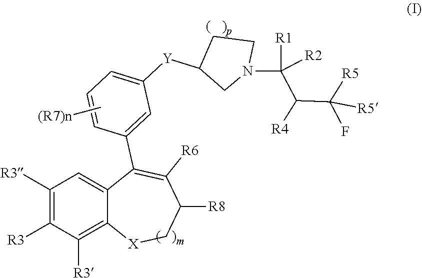

wherein:
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
R6 represents a group selected from:
   a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
   a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
   a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
   a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
   a cycloalkyl group comprising 3 to 7 carbon atoms, said cycloalkyl group being saturated or partially saturated and being optionally substituted with 1 to 4 substituents independently selected from:
      a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
      a (C₃-C₆)cycloalkyl group, and a phenyl group, said (C₃-C₆)cycloalkyl or phenyl groups being optionally substituted with one or two halogen atom(s) or (C₁-C₃)alkyl group(s);
   a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
   a 4 to 7 membered-heterocycloalkyl group comprising 1 or 2 heteroatoms independently selected from oxygen, nitrogen and sulfur, such as a tetrahydropyranyl or a tetrahydrofuranyl group, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
   a (C₁-C₆)alkyl group, such as an isobutyl group or an ethylbutyl group, said alkyl group being optionally substituted with 1 to 4 substituents independently selected from: a fluorine atom, a (C₁-C₃)alkoxy group, a (C₁-C₃)fluoroalkoxy group and a —OH group; and
   a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH₂—, —O— or —NH—;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
R8 represents a hydrogen atom or a (C₁-C₃)alkyl group or a cyclopropyl;
n is 0, 1 or 2;
m is 0 or 1; and
p is 0 or 1.

Disclosed herein are also compounds of the formula (I), or pharmaceutically acceptable salts thereof:

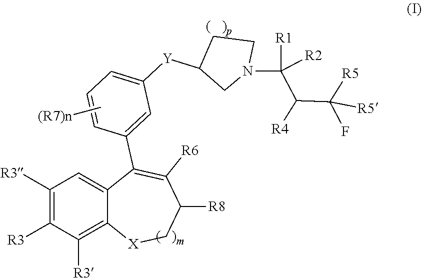

wherein:
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
R6 represents a group selected from:
   a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
   a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
   a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
   a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
   a cycloalkyl group comprising 3 to 7 carbon atoms, said cycloalkyl group being saturated or partially saturated and being optionally substituted with 1 to 4 substituents independently selected from:
      a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
      a (C₃-C₆)cycloalkyl group, and a phenyl group, said (C₃-C₆)cycloalkyl or phenyl groups being optionally substituted with one or two halogen atom(s) or (C₁-C₃)alkyl group(s);
   a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
   a 4 to 7 membered-heterocycloalkyl group comprising 1 or 2 heteroatoms independently selected from oxygen, nitrogen and sulfur, such as a tetrahydropyranyl or a tetrahydrofuranyl group, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
   a (C₁-C₆)alkyl group, such as an isobutyl group or an ethylbutyl group, said alkyl group being optionally substituted with 1 to 4 substituents independently selected from: a fluorine atom, a (C₁-C₃)alkoxy group, a (C₁-C₃)fluoroalkoxy group and a —OH group; and
   a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH₂—, —O— or —NH—;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
R8 represents a hydrogen atom, or a (C₁-C₃)alkyl group or a cyclopropyl;
n is 0, 1 or 2;
m is 0 or 1; and
p is 0 or 1;
   with the proviso that when Y represents —O— or —NH—, and p is 1, then the asymmetric carbon of the pyrrolidine linked to Y, is of (R) configuration.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), hydrochloride may be cited.

As used herein, the terms below have the following definitions unless otherwise mentioned throughout the instant specification:
a halogen atom: a fluorine, a chlorine, a bromine or an iodine atom, and in particular a fluorine and a chlorine atom;
an oxo: a “═O” group;
an alkyl group: a linear or branched saturated hydrocarbon-based aliphatic group comprising, unless otherwise mentioned, from 1 to 6 carbon atoms (noted “(C₁-C₆) alkyl”).

By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
a cycloalkyl group: a monocyclic alkyl group comprising, unless otherwise mentioned, from 3 to 7 carbon atoms, saturated or partially unsaturated and unsubstituted or substituted.

By way of examples, mention may be made of, but not limited to: cyclopropyl, cyclobutyl, cyclopentyl, cyclobutenyl, cyclopentenyl, cyclohexyl, cyclohexenyl, cycloheptyl, cycloheptenyl, groups and the like, in particular a cyclopentyl, a cyclohexyl, a cycloheptyl, a cycloheptenyl, or a cyclohexenyl;
a cycloalkylalkyl group: an alkyl group substituted with a cyclic alkyl group as defined above. Mention may be made of, but not limited to: cyclobutylmethyl;
a heterocycloalkyl group: a 4 to 7-membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl, tetrahydropyranyl, and tetrahydrothiopyranyl. The heterocycloalkyl is advantageously tetrahydrofuranyl or tetrahydropyranyl.
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of —CH₂F, —CHF₂, —CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named a (C₁-C₄)alkylsulfanyl group: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a fused phenyl group: a bicyclic radical comprising from 7 to 10 carbon atoms and that contains a phenyl moiety. Said phenyl moiety may be fused to a (C₃-C₆)cycloalkyl group, i.e. the phenyl moiety may share a bond with said (C₃-C₆)cycloalkyl group. The fused phenyl group may be bound to the rest of the molecule by its phenyl moiety. It may be substituted. Examples are, but are not limited to indanyl, bicyclo[4.2.0]octa-1(6),2,4-trienyl, tetrahydronaphthalenyl and the like;
a heteroaryl group: a cyclic 5 to 10-membered aromatic group containing between 2 and 9 carbon atoms and containing between 1 and 3 heteroatoms, such as nitrogen, oxygen or sulfur. Such nitrogen atom may be substituted with an oxygen atom in order to form a —N—O bond. Such —N—O bond can be in a form of a N-oxide (—N⁺—O—). Said heteroaryl group may be monocyclic or bicyclic. By way of examples of heteroaryl groups, mention may be made of, but not limited to: thiophene, furan, thiadiazole, thiazole, imidazole, pyridazine, triazine, pyrazine, oxadiazole, pyrazole, isothiazole, oxazole, isoxazole, pyridine, pyrimidine, benzotriazole, benzoxazole, pyrrolo[2,3-b]pyridine, benzimidazole, benzoxadiazole, benzothiazole, benzothiadiazole, benzofuran, indole, isoquinoline, indazole, benzisoxazole, benzisothiazole, pyridone groups and the like. The heteroaryl group is advantageously pyridine, pyrrole, imidazole, pyrazine, furane, thiazole, pyrazole, thiadiazole, pyridazine, pyridone and pyrimidine, and more particularly pyridine, pyridone and pyrrole;
a bicyclic group, generally comprising 5 to 12 carbon atoms, is a hydrocarbon group selected from groups comprising two rings connected through:
a single common atom: a “spirobicyclic ring”. Such spiro bicyclic alkyl generally comprises 5 to 11 carbon atoms referring to a “spiro(C₅-C₁₁)bicyclic ring”. The rings may be saturated or partially unsaturated. Such spirobicyclic ring may be unsubstituted or substituted, in particular by at least one (C₁-C₃)alkyl group such as methyl or a fluorine. By way of examples of spiro(C₅-C₁₁)bicyclic ring as for the definition of R6, mention may be made of, but not limited to: spiro[2.3]hexane, spiro[3.3]heptane, spiro[3.3]heptene, spiro[2.5]octane and 7-azaspiro[3.5]nonane. The spiro(C₅-C₁₁)bicyclic ring is advantageously spiro[3.3]heptane or spiro[3.3]heptene still for the R6 group.
two common atoms: In that case the bicyclic group comprises 7 to 12 carbon atoms and optionally comprises 1 to 2 unsaturations. By way of examples of such bicyclic groups, mention may be made of, but not limited to: cis-1,3a,4,5,6,6a-hexahydropentalenyl group, bicyclo[3.1.0]hexan-1-yl, bicyclo[4.1.0]heptanyl and octahydropentalenyl.
three or more common atoms: In that case the bicyclic group comprises 6 to 10 carbon atoms, such bicyclic group may be referred to as a “bridged (C₆-C₁₀)cycloalkyl” group, the rings share three or more atoms and the bridge contains at least one atom, for example 1, 2 or 3 atoms and preferentially 1 atom. By way of examples of such bridged cycloalkyl groups, mention may be made of, but not limited to bicyclo[3.2.1]octan-3-yl and bicyclo[2.2.1]heptan-2-yl.
a zwitterion means: a globally neutral molecule with a positive and a negative electrical charge and having an acidic group and a basic group.

In another embodiment, in the compounds of formula (I) as defined above, the asymmetric carbon of the pyrrolidine or of the azetidine, linked to Y, is of (R) configuration.

In another embodiment, in the compounds of formula (I) as defined above, R1 and R2 are a hydrogen atom.

In another embodiment, in the compounds of formula (I) as defined above, R3 is a —COOH group.

In another embodiment, in the compounds of formula (I) as defined above, R3 is a hydrogen atom.

In another embodiment, in the compounds of formula (I) as defined above, R3 is a —OH group.

In another embodiment, in the compounds of formula (I) as defined above, R3′ and R3″ represent a hydrogen atom.

In another embodiment, in the compounds of formula (I) as defined above, R4 represents a hydrogen atom.

In another embodiment, in the compounds of formula (I) as defined above, X represents —O— or —CH₂—, and preferably X represents —CH₂—.

In another embodiment, in the compounds of formula (I) as defined above, X represents —CH₂—.

In another embodiment, in the compounds of formula (I) as defined above, X represents —O—.

In another embodiment, in the compounds of formula (I) as defined above, R5 and R5′ represent a hydrogen atom.

In another embodiment, in the compounds of formula (I) as defined above, R6 represents:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group; or
a cycloalkyl group comprising 3 to 7 carbon atoms, said cycloalkyl group being saturated or partially saturated and being optionally substituted with 1 to 4 substituents independently selected from:
   a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
   a (C₃-C₆)cycloalkyl group, and a phenyl group, said (C₃-C₆)cycloalkyl or phenyl groups being optionally substituted with one or two halogen atom(s) or (C₁-C₃)alkyl group(s).

In another embodiment, in the compounds of formula (I) as defined above, R6 represents a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group.

In another embodiment, in the compounds of formula (I) as defined above, R6 represents a phenyl group, said phenyl group being optionally substituted with 1 or 2 substituents independently selected from a chlorine atom, a fluorine atom, and a methyl group.

In another embodiment, in the compounds of formula (I) as defined above, R6 represents a phenyl group.

In another embodiment, in the compounds of formula (I) as defined above, R6 represents a phenyl group, said phenyl group being substituted with 1 or 2 substituents independently selected from a chlorine atom, a fluorine atom, and a methyl group.

In another embodiment, in the compounds of formula (I) as defined above, R6 represents a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group, and a phenyl group, said (C₃-C₆)cycloalkyl or phenyl groups being optionally substituted with one or two halogen atom(s) or (C₁-C₃)alkyl group(s).

In another embodiment, in the compounds of formula (I) as defined above, R6 represents a cyclohexyl group, said cyclohexyl group being substituted with 1 or 2 substituents independently selected from a chlorine atom and a methyl group.

In another embodiment, in the compounds of formula (I) as defined above, R6 represents a cyclopentyl group.

In another embodiment, in the compounds of formula (I) as defined above, R7 represents a methyl group or a fluorine atom and n is 0 or 1.

In another embodiment, in the compounds of formula (I) as defined above, R8 represents a hydrogen atom or a methyl group.

In another embodiment, in the compounds of formula (I) as defined above, Y represents —O—.

In another embodiment, in the compounds of formula (I) as defined above, Y represents —O—, and the asymmetric carbon of the pyrrolidine or of the azetidine linked to —O— is of (R) configuration.

In another embodiment, in the compounds of formula (I) as defined above, Y represents —O—, p is 1, and the asymmetric carbon of the pyrrolidine linked to —O— is of (R) configuration.

In another embodiment, in the compounds of formula (I) as defined above, Y represents —CH₂—.

In another embodiment, in the compounds of formula (I) as defined above, Y represents —NH—.

In another embodiment, in the compounds of formula (I) as defined above, Y represents —NH—, and the asymmetric carbon of the pyrrolidine or of the azetidine linked to —NH— is of (R) configuration.

In another embodiment, in the compounds of formula (I) as defined above, Y represents —NH—, p is 1, and the asymmetric carbon of the pyrrolidine linked to —NH— is of (R) configuration.

In another embodiment, in the compounds of formula (I) as defined above, m is 1.

In another embodiment, in the compounds of formula (I) as defined above, m is 0.

In another embodiment, in the compounds of formula (I) as defined above, p is 1.

In another embodiment, in the compounds of formula (I) as defined above, p is 0.

In another embodiment, in the compounds of formula (I) as defined above, R3 is a —COOH group and R6 is a phenyl group, said phenyl group being optionally substituted with 1 or 2 substituents independently selected from a chlorine atom, a fluorine atom, and a methyl group. In such embodiment, R3′ and R3″ are in particular hydrogen atoms. Still in such embodiment, R1, R2, R4, R5, R5′ and R8 are hydrogen atoms. In such embodiment, Y is —O—, p is equal to 1, m is equal to 1 and n is equal to 0. Still in such embodiment, X is a —CH₂— group.

In another embodiment, in the compounds of formula (I) as defined above, R3 is a —COOH group and R6 is a cyclohexyl group, said cyclohexyl group being substituted with 1 or 2 substituents independently selected from a chlorine atom and a methyl group. In such embodiment, R3′ and R3″ are in particular hydrogen atoms. Still in such embodiment, R1, R2, R4, R5, R5′ and R8 are hydrogen atoms. In such embodiment, Y is —O—, p is equal to 1, m is equal to 1 and n is equal to 0. Still in such embodiment, X is a —CH₂— group.

In another embodiment, in the compounds of formula (I) as defined above, R3 is a —COOH group and R6 is a cyclopentyl group. In such embodiment, R3′ and R3″ are in particular hydrogen atoms. Still in such embodiment, R1, R2, R4, R5, R5′ and R8 are hydrogen atoms. In such embodiment, Y is —O—, p is equal to 1, mis equal to 1 and n is equal to 0. Still in such embodiment, X is a —CH₂— group.

Among the compounds of formula (I) described herein, mention may be made in particular of the following compounds or a pharmaceutically acceptable salt thereof, in particular hydrochloride salt thereof:
(R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylic acid, (1)
(R)-9-(3-((1-(3-fluoropropyl)pyrrolidin-3-yl)oxy)phenyl)-8-phenyl-6,7-dihydro-5H-benzo[7]annulene-3-carboxylic acid, (2)
(R)-8-(2-chlorophenyl)-9-(3-((1-(3-fluoropropyl)pyrrolidin-3-yl)oxy)phenyl)-6,7-dihydro-5H-benzo[7]annulene-3-carboxylic acid, (3)
(R)-8-(2-chloro-4-methylphenyl)-9-(3-((1-(3-fluoropropyl)pyrrolidin-3-yl)oxy)phenyl)-6,7-dihydro-5H-benzo[7]annulene-3-carboxylic acid, (4)
(R)-8-(2-chloro-4-fluorophenyl)-9-(3-((1-(3-fluoropropyl)pyrrolidin-3-yl)oxy)phenyl)-6,7-dihydro-5H-benzo[7]annulene-3-carboxylic acid, (5)
(R)-8-(4-chloro-2-fluorophenyl)-9-(3-((1-(3-fluoropropyl)pyrrolidin-3-yl)oxy)phenyl)-6,7-dihydro-5H-benzo[7]annulene-3-carboxylic acid, (6)
(R)-8-(2-fluoro-4-methylphenyl)-9-(3-((1-(3-fluoropropyl)pyrrolidin-3-yl)oxy)phenyl)-6,7-dihydro-5H-benzo[7]annulene-3-carboxylic acid, (7)
(R)-8-(2,4-dimethylphenyl)-9-(3-((1-(3-fluoropropyl)pyrrolidin-3-yl)oxy)phenyl)-6,7-dihydro-5H-benzo[7]annulene-3-carboxylic acid, (8)
(R)-8-(2-chloro-3-fluorophenyl)-9-(3-((1-(3-fluoropropyl)pyrrolidin-3-yl)oxy)phenyl)-6,7-dihydro-5H-benzo[7]annulene-3-carboxylic acid, (9)
(R)-8-(4,4-difluorocyclohexyl)-9-(3-((1-(3-fluoropropyl)pyrrolidin-3-yl)oxy)phenyl)-6,7-dihydro-5H-benzo[7]annulene-3-carboxylic acid hydrochloride, (10)
(R)-9-(3-((1-(3-fluoropropyl)pyrrolidin-3-yl)oxy)phenyl)-8-(4-methylcyclohexyl)-6,7-dihydro-5H-benzo[7]annulene-3-carboxylic acid, Isomer 1, (11)
(R)-9-(3-((1-(3-fluoropropyl)pyrrolidin-3-yl)oxy)phenyl)-8-(4-methylcyclohexyl)-6,7-dihydro-5H-benzo[7]annulene-3-carboxylic acid, Isomer 2, (12)
(R)-8-(4,4-dimethylcyclohexyl)-9-(3-((1-(3-fluoropropyl)pyrrolidin-3-yl)oxy)phenyl)-6,7-dihydro-5H-benzo[7]annulene-3-carboxylic acid, hydrochloride, (13)
(R)-8-cyclopentyl-9-(3-((1-(3-fluoropropyl)pyrrolidin-3-yl)oxy)phenyl)-6,7-dihydro-5H-benzo[7]annulene-3-carboxylic acid, (14)
(R)-9-(3-((1-(3-fluoropropyl)pyrrolidin-3-yl)oxy)-2-methylphenyl)-8-phenyl-6,7-dihydro-5H-benzo[7]annulene-3-carboxylic acid, (15)
(R)-8-(2-chlorophenyl)-9-(3-((1-(3-fluoropropyl)pyrrolidin-3-yl)oxy)-2-methylphenyl)-6,7-dihydro-5H-benzo[7]annulene-3-carboxylic acid, (16)
(R)-8-(2,4-dichlorophenyl)-9-(3-((1-(3-fluoropropyl)pyrrolidin-3-yl)oxy)-2-methylphenyl)-6,7-dihydro-5H-benzo[7]annulene-3-carboxylic acid, (17)
(R)-8-(4-fluoro-2-methylphenyl)-9-(3-((1-(3-fluoropropyl)pyrrolidin-3-yl)oxy)-2-methylphenyl)-6,7-dihydro-5H-benzo[7]annulene-3-carboxylic acid, (18)
(R)-9-(2-fluoro-3-((1-(3-fluoropropyl)pyrrolidin-3-yl)oxy)phenyl)-8-phenyl-6,7-dihydro-5H-benzo[7]annulene-3-carboxylic acid, hydrochloride, (19)
(R)-8-(2-chlorophenyl)-9-(2-fluoro-3-((1-(3-fluoropropyl)pyrrolidin-3-yl)oxy)phenyl)-6,7-dihydro-5H-benzo[7]annulene-3-carboxylic acid, hydrochloride, (20)
(R)-8-(2,4-dichlorophenyl)-9-(2-fluoro-3-((1-(3-fluoropropyl)pyrrolidin-3-yl)oxy)phenyl)-6,7-dihydro-5H-benzo[7]annulene-3-carboxylic acid, hydrochloride, (21)
(R)-9-(5-((1-(3-fluoropropyl)pyrrolidin-3-yl)oxy)-2-methylphenyl)-8-phenyl-6,7-dihydro-5H-benzo[7]annulene-3-carboxylic acid, (22)
(R)-8-(2-chlorophenyl)-9-(5-((1-(3-fluoropropyl)pyrrolidin-3-yl)oxy)-2-methylphenyl)-6,7-dihydro-5H-benzo[7]annulene-3-carboxylic acid, (23)
(R)-8-(2-chlorophenyl)-9-(5-((1-(3-fluoropropyl)pyrrolidin-3-yl)oxy)-2-methylphenyl)-6,7-dihydro-5H-benzo[7]annulene-3-carboxylic acid, (24)
(R)-9-(2-fluoro-5-((1-(3-fluoropropyl)pyrrolidin-3-yl)oxy)phenyl)-8-phenyl-6,7-dihydro-5H-benzo[7]annulene-3-carboxylic acid, hydrochloride, (25)
(R)-8-(2-chlorophenyl)-9-(2-fluoro-5-((1-(3-fluoropropyl)pyrrolidin-3-yl)oxy)phenyl)-6,7-dihydro-5H-benzo[7]annulene-3-carboxylic acid, hydrochloride, (26)
(R)-8-(2,4-dichlorophenyl)-9-(2-fluoro-5-((1-(3-fluoropropyl)pyrrolidin-3-yl)oxy)phenyl)-6,7-dihydro-5H-benzo[7]annulene-3-carboxylic acid hydrochloride, (27)
8-(2,4-dichlorophenyl)-9-(3-((1-(3-fluoropropyl)pyrrolidin-3-yl)methyl)phenyl)-6,7-dihydro-5H-benzo[7]annulene-3-carboxylic acid, Isomer 1, (28)
8-(2-chlorophenyl)-9-(3-((1-(3-fluoropropyl)azetidin-3-yl)oxy)phenyl)-6,7-dihydro-5H-benzo[7]annulene-3-carboxylic acid hydrochloride, (29)
8-(2,4-dichlorophenyl)-9-(3-((1-(3-fluoropropyl)azetidin-3-yl)oxy)phenyl)-6,7-dihydro-5H-benzo[7]annulene-3-carboxylic acid hydrochloride, (30)
8-(2,4-dichlorophenyl)-9-(3-((1-(3-fluoropropyl)azetidin-3-yl)methyl)phenyl)-6,7-dihydro-5H-benzo[7]annulene-3-carboxylic acid, (31)
8-(2,4-dichlorophenyl)-9-(3-((1-(3-fluoropropyl)azetidin-3-yl)amino)phenyl)-6,7-dihydro-5H-benzo[7]annulene-3-carboxylic acid, (32)
(R)-8-(2,4-dichlorophenyl)-9-(3-((1-(3-fluoropropyl)pyrrolidin-3-yl)amino)phenyl)-6,7-dihydro-5H-benzo[7]annulene-3-carboxylic acid hydrochloride, (33)
(R)-8-(2,4-dichlorophenyl)-9-(3-((1-(3-fluoropropyl)pyrrolidin-3-yl)oxy)phenyl)-6,7-dihydro-5H-benzo[7]annulen-3-ol, (34)
(R)-4-(2,4-dichlorophenyl)-5-(3-((1-(3-fluoropropyl)pyrrolidin-3-yl)oxy)phenyl)-2,3-dihydrobenzo[b]oxepine-8-carboxylic acid hydrochloride, (35)
8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylic acid, Isomer 1, (36)
8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylic acid, Isomer 2, (37)
(R)-3-(3-(8-(2,4-dichlorophenyl)-6,7-dihydro-5H-benzo[7]annulen-9-yl)phenoxy)-1-(3-fluoropropyl)pyrrolidine, (38)
(R)-3-(3-(8-(2chlorophenyl)-6,7-dihydro-5H-benzo[7]annulen-9-yl)phenoxy)-1-(3-fluoropropyl)pyrrolidine, (39)
(R)-6-(2,4-dichlorophenyl)-5-(3-((1-(3-fluoropropyl)pyrrolidin-3-yl)oxy)phenyl)-7,8-dihydronaphthalene-2-carboxylic acid hydrochloride, (40), and
(R)-4-(2,4-dichlorophenyl)-5-(3-((1-(3-fluoropropyl)pyrrolidin-3-yl)oxy)phenyl)-2,3-dihydrobenzo[b]thiepin-8-ol, (41).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) optionally modified by the use of appropriate reagents and conditions for the introduction of the various moieties found in the formula (I) as described below.

The following abbreviations and empirical formulae are used:
Ar Argon
BOC₂O di-ter-butyldicarbonate
CO Carbon monoxide
Cs₂CO₃ Cesium carbonate
DABCO 1,4-diazabicyclo[2.2.2]octane
DBU 1,8-Diazabicyclo[5.4.0]undec-7-ene
DCM Dichloromethane
DIEA Diisopropylethylamine
DMF N,N-dimethylformamide
DMAP Dimethylaminopyridine
DMSO Dimethyl sulfoxide
Et₂O Diethyl ether
EtOAc Ethyl acetate
EtOH Ethanol
H₂ Hydrogen
HCl Hydrochloric acid
HPLC High performance liquid chromatography
K₂CO₃ Potassium carbonate
KHMDS Potassium hexamethyldisilazane
KOH Potassium hydroxide
LiHMDS Lithium bis(trimethylsilyl)amide
LiOH Lithium hydroxide
MeCN Acetonitrile
MeOH Methanol
MeTHF 2-Methyltetrahydrofuran
MgSO₄ Magnesium sulfate
MTBE Methyl tert-butyl ether
n-BuLi n-Butyllithium
Na₂SO₄ Sodium sulfate
NaH Sodium hydride
NaHCO₃Sodium bicarbonate
NaOH Sodium hydroxide
NH₄OH Ammonium hydroxide
Pd₂(dba)₃ tris(dibenzylideneacetone)dipalladium(0)
Pd/C Palladium on carbon
Pd(dppf)Cl₂ [1,1′-Bis(diphenylphosphino)ferrocene]dichloropalladium(II)
Pd(OAc)₂ Palladium acetate
Pd(PPh₃)₂Cl₂ bis(triphenylphosphine) palladium(II) dichloride
PhOK Potassium phenolate
PPh₃ Triphenylphosphine
RT Room temperature
SCX Strong cation exchange
SFC Supercritical Fluid Chromatography
TEA Triethylamine
TFA Trifluoroacetic acid
THF Tetrahydrofuran
XANTPHOS (9,9-dimethyl-9H-xanthene-4,5-diyl)bis(diphenylphosphane)

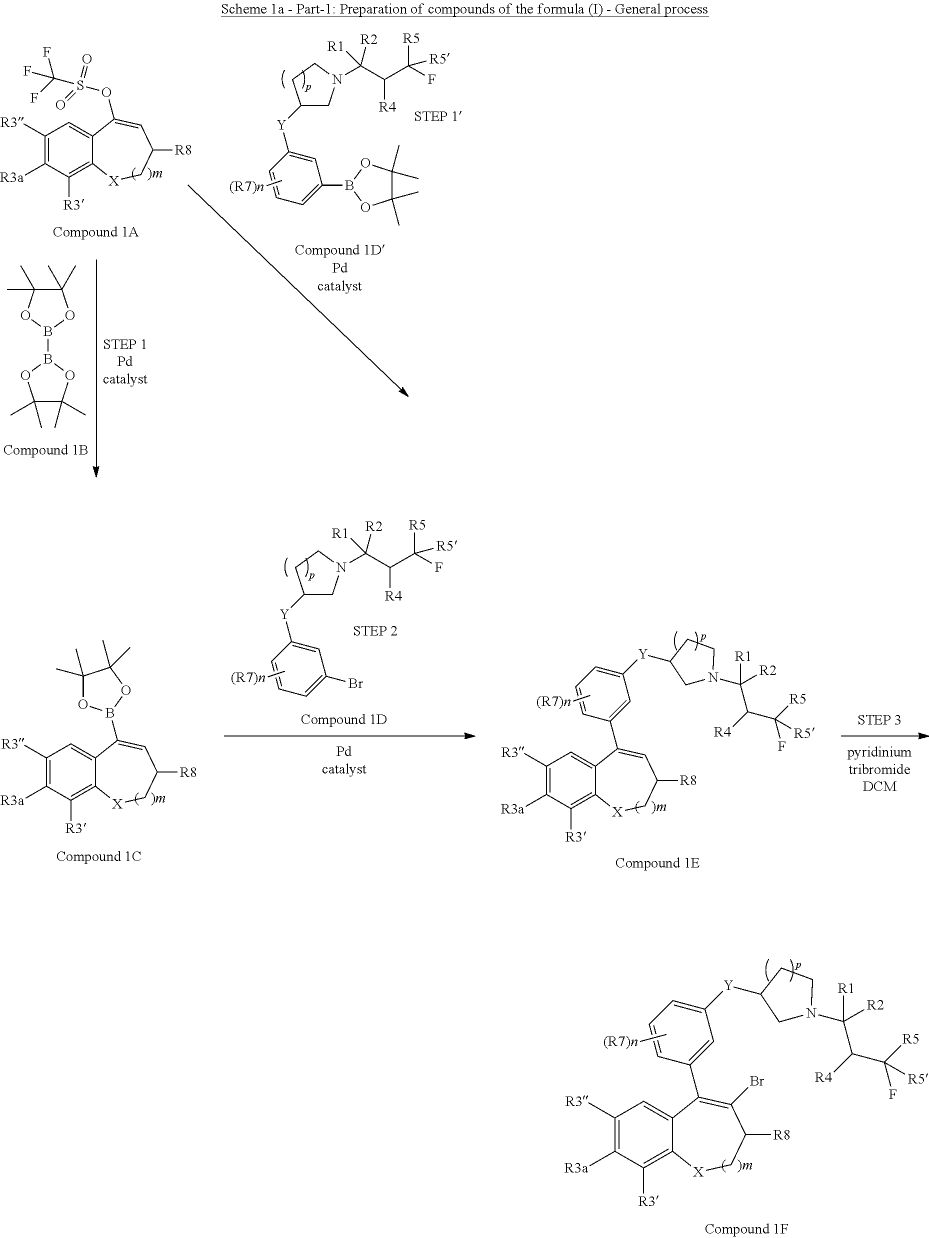

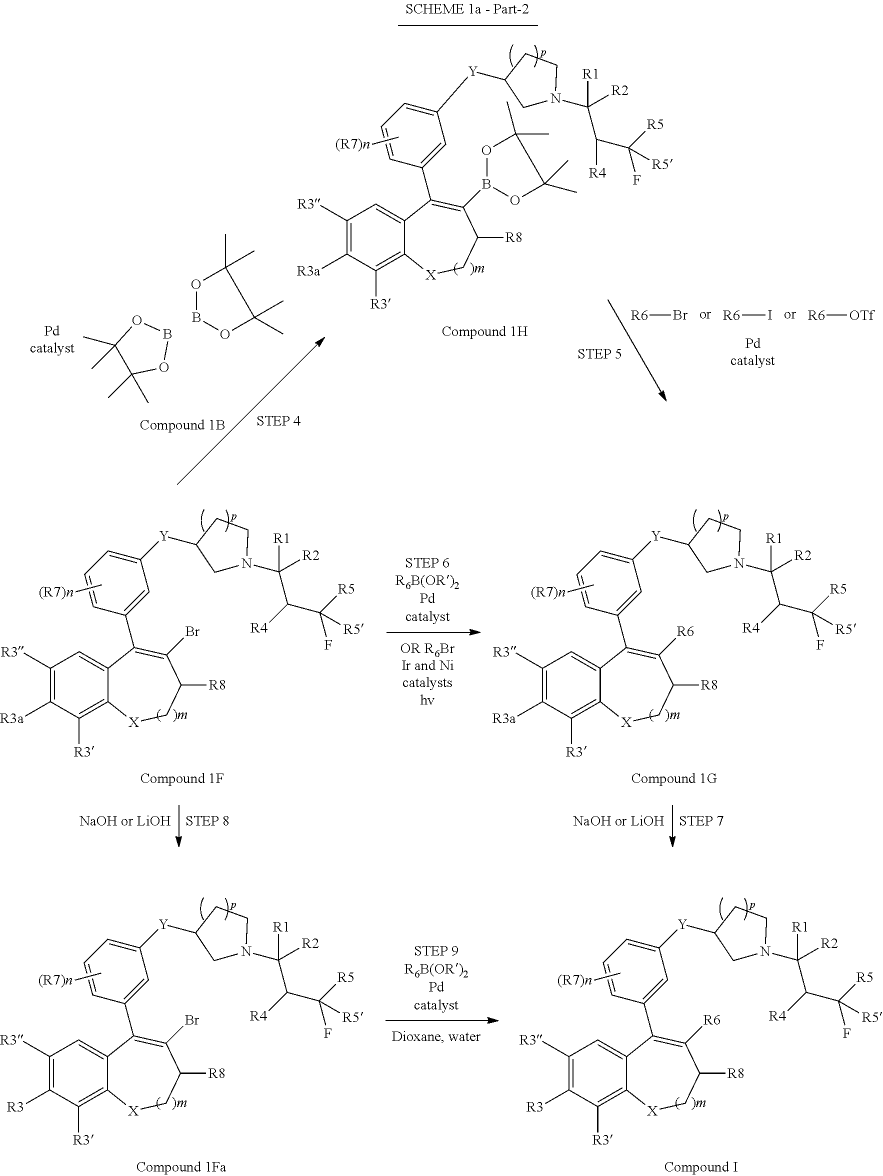

According to SCHEME 1a—Part-1 and Part-2, in which R3a is a hydrogen atom or a carboxylic ester such as —COOMe, —COOEt, or protected —OH such as O-pivaloyl for example, R1, R2, R3, R3′, R3″, R4, R5, R5′, R6, R7, R8, X, m, n, p and Y are defined as described above, compound 1A (prepared according to WO2017140669 when X=C) can be converted in STEP 1 to compound 1C by treatment with compound 1B in the presence of a palladium catalyst, for example bis(triphenylphosphine)palladium(II) dichloride Pd(PPh₃)₂Cl₂, and a phosphine such as triphenylphosphine in solution in toluene by heating up to reflux of solvent, in presence of a base such as KOPh.

Compound 1C can be converted in STEP 2 to compound 1E by treatment with compound 1D in a Suzuki coupling reaction using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃) by heating up to reflux of solvent.

Alternatively, compound 1E can be obtained in STEP 1′ by Suzuki coupling between compound 1A and compound 1D′ using for example [1,1′-bis(diphenylphosphino) ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1E can be converted in STEP 3 to compound 1F by treatment for example with pyridinium tribromide in DCM or THF at room temperature.

Compound 1F can be converted in STEP 4 to compound 1H by treatment with compound 1B in the presence of a palladium catalyst, for example bis(triphenylphosphine) palladium(II) dichloride Pd(PPh₃)₂Cl₂, and a phosphine such as triphenylphosphine in solution in toluene by heating up to reflux of solvent in presence of a base such as KOPh or AcOK.

Compound 1G wherein R6 is phenyl or heteroaryl can be prepared in a Suzuki coupling reaction between compounds 1H and R6Br or R6I or R6OTf in STEP 5 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1G can also be prepared from compound 1F in STEP 6 in a Suzuki coupling with a suitable boronic reagent R6B(OR′)₂, wherein —B(OR′)₂ is a boronic acid or a pinacolate ester and R6 is defined as above, using for example Pd(dppf)Cl₂, complex with DCM, as catalyst, in a mixture of dioxane and water as solvent and in the presence of a base, for example Cs₂CO₃, at room temperature or by heating up to reflux of solvent. When R6 is a substituted cycloalkene, heterocycloalkene, it may be reduced by hydrogenation with a catalyst such as Pd/C under hydrogen pressure (H₂) around 5 bars for example at temperature up to 70° C. to give the corresponding saturated compound 1G.

Alternatively, compound 1F can be subjected to a photocatalyzed coupling reaction with R6Br, where R6 is an alkyl group, a cycloalkyl or a spiro bicyclic alkyl as defined above, using catalysts such as (Ir[dF(CF₃)ppy]₂(dtbpy))PF₆ and nickel(II) chloride ethylene glycol dimethyl ether complex in presence of tris(trimethylsilyl)silane and bases such as 4,4′-di-tert-butyl-2,2′-bipyridine and sodium carbonate, to give the corresponding compound 1G.

Compound 1G can be converted in STEP 7 to compound of formula (I) in presence of a source of hydroxide ions such as NaOH or LiOH in solution in methanol (MeOH).

Compound 1F can be converted in STEP 8 to compound 1Fa in the presence of a source of hydroxide ions such as NaOH or LiOH in solution in methanol (MeOH).

This compound 1Fa can be converted in STEP 9 to compound of formula (I) through Suzuki conditions using a suitable boronic reagent R6B(OR′)₂, wherein —B(OR′)₂ is a boronic acid or a pinacolate ester and R6 is as above defined, using for example Pd(dppf)Cl₂, complex with DCM, as catalyst, in a mixture of dioxane and water as solvent and in the presence of a base, for example Cs₂CO₃, at room temperature or by heating up to reflux of solvents. When R6 is a substituted cycloalkene, heterocycloalkene, it may be reduced by hydrogenation with a catalyst, such as Pd/C under hydrogen (H₂) pressure around 5 bars, for example at temperature up to 70° C., to give the corresponding saturated compound of formula (I).

When R3a is —COOMe, —COOEt, or a protected —OH such as O-pivaloyl, deprotection can be performed in STEP 7 by treatment with an aqueous solution of sodium hydroxide (NaOH) 2N or lithium hydroxide (LiOH) in methanol (MeOH). When R3 is —COOH, extraction of the product can give the sodium or lithium salt of compound of formula (I). The acidification with an aqueous solution of HCl 2N to pH 6-7 can give the neutral form of compound of formula (I). The acidification with an aqueous solution of HCl 2N to pH 1-2 can give the hydrochloride salt of compound of formula (I). The purification using HPLC in presence of formic acid or trifluoroacetic acid in the eluent can give the formate or trifluoroacetate salt of compound of formula (I).

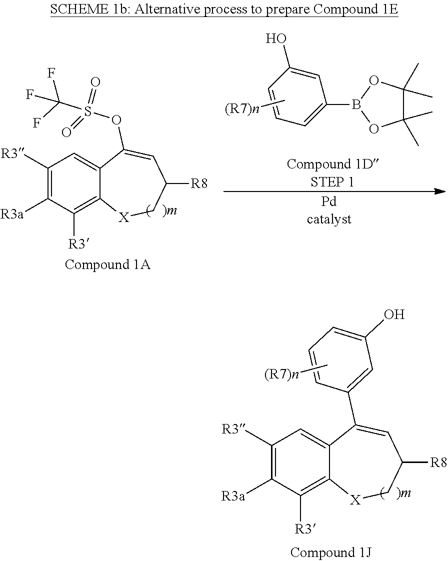

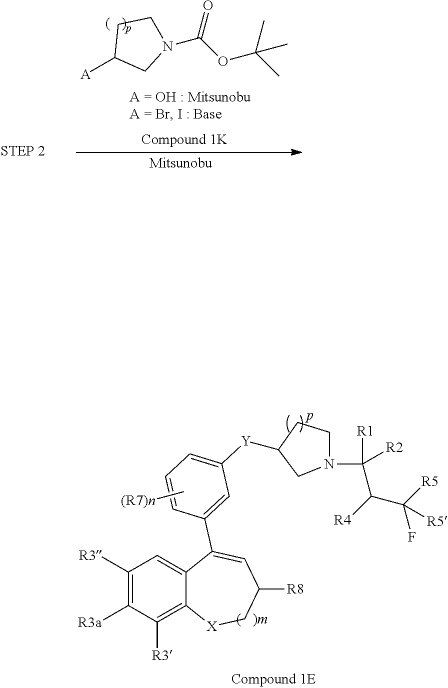

According to SCHEME 1b, in which R3a is a hydrogen atom or a carboxylic ester such as —COOMe, —COOEt, or protected —OH such as 0-pivaloyl for example, R1, R2, R3a, R3′, R3″, R4, R5, R5′, R7, R8, X, m, n, and p are defined as described above and Y is —O—, compound 1A (prepared according to WO2017140669 when X=C), can be converted in STEP 1 to compound 1J by treatment for example with compound 1D″ using a palladium catalyst, for example bis(triphenylphosphine)palladium(II) dichloride Pd(PPh₃)₂Cl₂, in a mixture of dioxane and water as solvent and in the presence of a base, for example cesium carbonate (Cs₂CO₃) at room temperature or by heating up to reflux of solvents.

Compound 1J can be converted in STEP 2 to compound 1E (Y=—O—) by reaction with compound 1K (A=—Br or —I) in presence of a base such as cesium carbonate (Cs₂CO₃) in DMF or MeCN, or under Mitsunobu conditions, when (A=—OH), using for example N,N,N′,N′-tetramethylazodicarboxamide and triphenylphosphine as coupling agents in THF as a solvent.

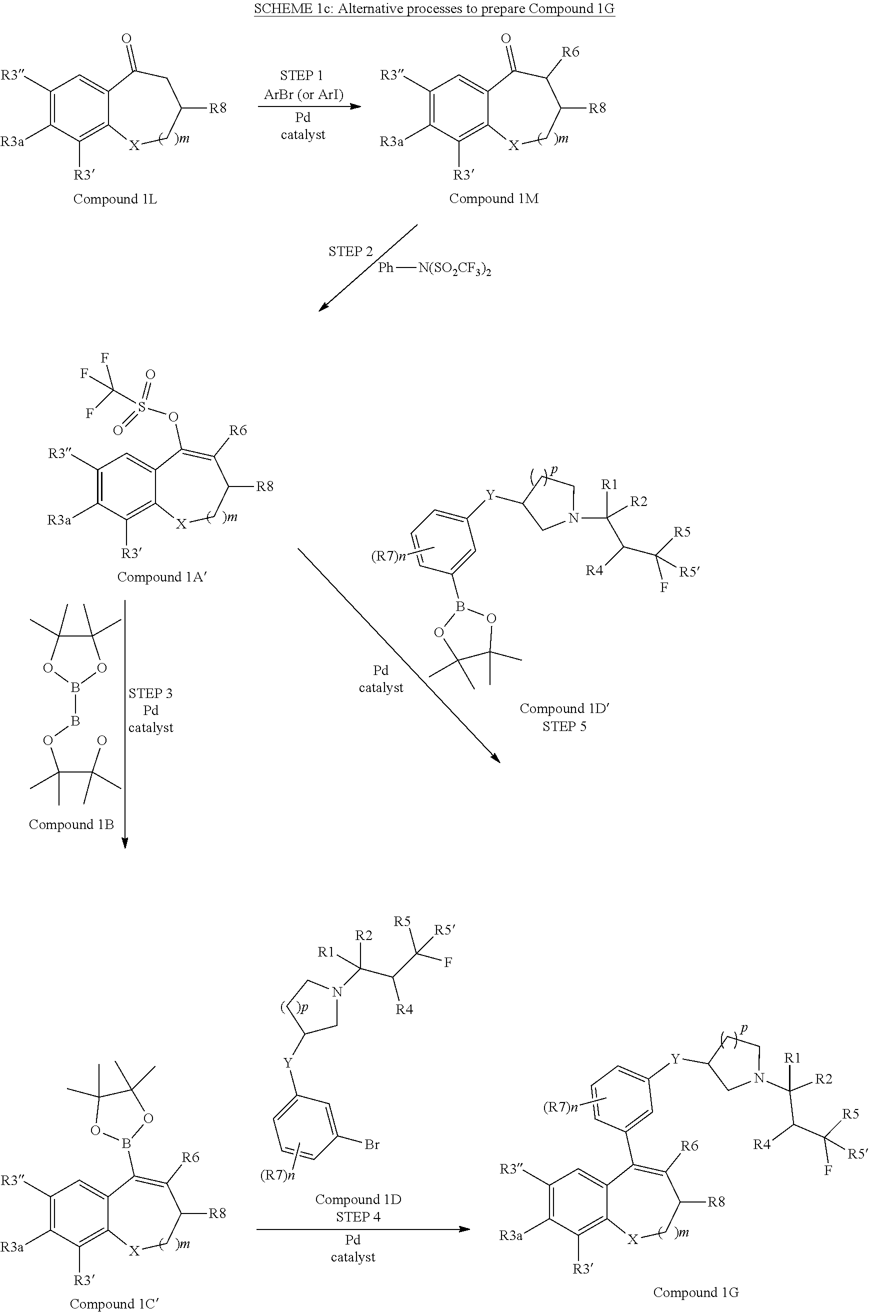

According to SCHEME 1c, in which R3a is a hydrogen atom or a carboxylic ester such as —COOMe, —COOEt, or protected —OH such as 0-pivaloyl for example, R1, R2, R3a, R3′, R3″, R4, R5, R5′, R7, R8, X, Y, m, n, and p are defined as described above, and R6 is phenyl or heteroaryl, compound 1L can be converted in STEP 1 to compound 1M by treatment with aryl and heteroaryl bromide or iodide in the presence of a palladium catalyst, for example tris(dibenzylideneacetone)dipalladium(0) Pd₂(dba)₃, and a phosphine such as (9,9-dimethyl-9H-xanthene-4,5-diyl)bis(diphenylphosphane) (XANTPHOS) in solution in toluene or xylene by heating up to reflux of solvent, in presence of a base such as K₂CO₃ or Cs₂CO₃. Alternative way to prepare compound 1M, wherein R6 can be any of the groups defined above for R6 in formula (I), is described in SCHEME if below.

Compound 1M can be converted in STEP 2 to compound 1A′ by treatment with N,N-bis(trifluoromethylsulfonyl)aniline in the presence of base such as DBU or NaH, or KHMDS at −50° C., in a solvent such as MeTHF.

Compound 1A′ can be converted in STEP 3 to compound IC′ by treatment with compound 1B and with a palladium catalyst, for example bis(triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1G can be prepared in a Suzuki coupling reaction either between compounds 1C′ and 1D in STEP 4 or between compounds 1A′ and 1D′ in STEP 5 using for example [1,1′-bis(diphenylphosphino)ferrocene]dichloropalladium(II) (Pd(dppf)Cl₂), complex with DCM, as catalyst, in a mixture of dioxane and water and in the presence of a base, for example Cs₂CO₃, by heating up to reflux of solvent.

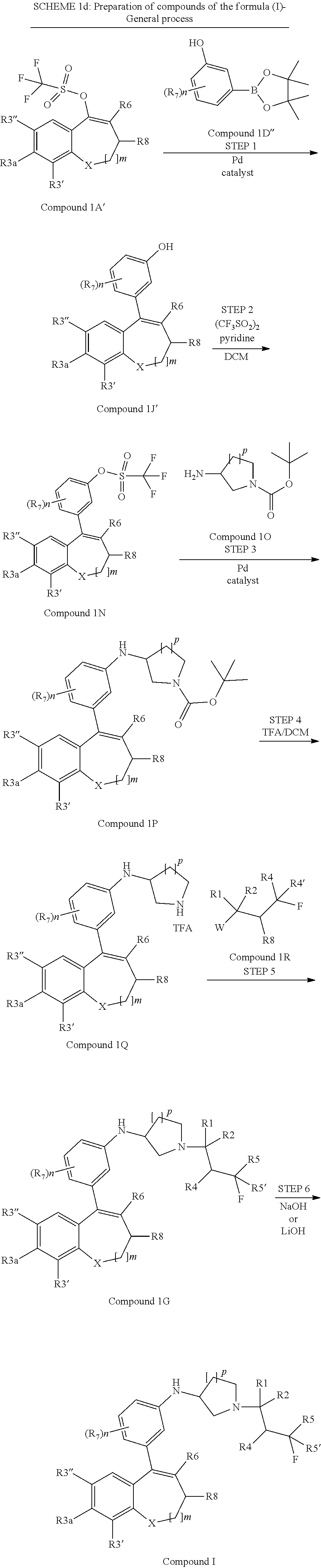

According to SCHEME id, in which R3a is a hydrogen atom or a carboxylic ester such as —COOMe, —COOEt, or protected —OH such as O-pivaloyl for example, R1, R2, R3, R3′, R3″, R4, R5, R5′, R6, R7, R8, X, m, n, and p are defined as described above and Y is —NH—, Compound 1A′ can be converted in STEP 1 to compound 1J′ by treatment with compound 1D″ using for example [1,1′-bis(diphenylphosphino)ferrocene]dichloropalladium(I) (Pd(dppf)Cl₂), complex with DCM, as catalyst, in a mixture of dioxane and water and in the presence of a base, for example Cs₂CO₃, by heating up to reflux of solvent.

Compound 1J′ can be converted in STEP 2 to compound 1N by treatment with trifluoromethanesulfonic anhydride in the presence of pyridine in DCM.

Compound 1N can be converted in STEP 3 to compound 1P in a Buchwald coupling conditions with compound 10 in dioxane at 130° C. in microwave using for example xantphos and Pd(OAc)₂ as catalytic system.

Compound 1P can be converted in STEP 4 to compound 1Q by treatment with TFA in DCM.

Compound 1Q can be converted in STEP 5 to compound 1G by treatment with compound 1R, wherein W is Br, I or OSO₂R with R=CH₃, PhMe, CF₃ or CF₂CF₂CF₂CF₃, in presence of a base such as K₂CO₃ in DMF at 70° C. or in presence of NaOH or KOH in THF at room temperature or in presence of aqueous NaOH in DCM at room temperature.

When R3a is —COOMe, —COOEt, or a protected —OH such as O-pivaloyl, deprotection can be performed in STEP 6 by treatment with an aqueous solution of NaOH 2N or LiOH in MeOH. When R3 is —COOH, extraction of the product can give the sodium or lithium salt of compound of formula (I). The acidification with an aqueous solution of HCl 2N to pH 6-7 can give the neutral form of compound of formula (I). The acidification with an aqueous solution of HC 2N to pH 1-2 can give the hydrochloride salt of compound of formula (I). The purification using HPLC in presence of formic acid or trifluoroacetic acid in the eluent can give the formate or trifluoroacetate salt of compound of formula (I).

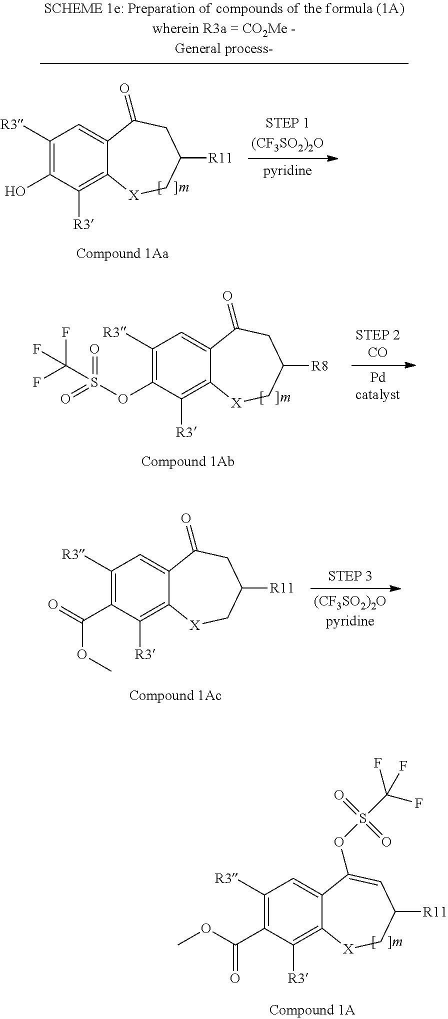

According to SCHEME 1e, in which X, m, R3′, R3″ and R8 are defined as described above, compound 1A can be prepared as follows: compound 1Aa (commercially available or prepared according to WO2017140669 and WO2018091153) can be converted in STEP 1 to compound 1Ab by treatment with trifluoromethanesulfonic anhydride, in solution in DCM, in the presence of pyridine as a base.

Compound 1Ab can be converted in STEP 2 to compound 1Ac by carbonylation with carbon monoxide, in solution in DMF and MeOH, in the presence of a palladium catalyst, for example [1,1′-bis(diphenylphosphino)ferrocene]dichloropalladium(II) (Pd(dppf)Cl₂), complex with DCM.

Compound 1Ac can be converted in STEP 3 to compound 1A wherein R3a=CO₂Me by treatment with trifluoromethanesulfonic anhydride, in solution in DCM, in the presence of pyridine as a base.

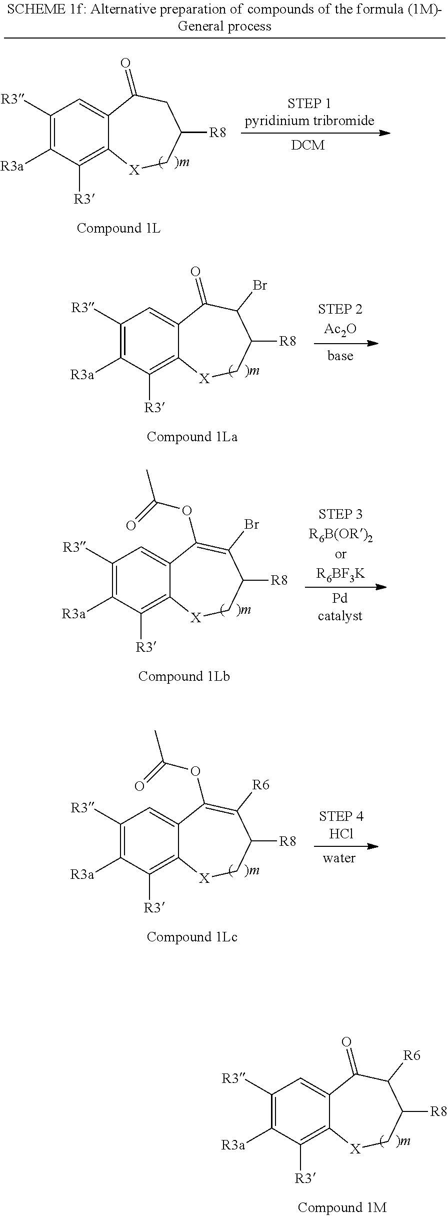

According to SCHEME if, in which R3a is a hydrogen atom, a carboxylic ester such as COOMe, COOEt, or protected OH such as O-pivaloyl for example, R3′, R3″, R8, R6, X and m are defined as described above, compound 1M can alternatively be prepared as follows: compound 1L can be converted in STEP 1 to compound 1La by treatment with pyridinium tribromide in DCM or THF at room temperature for example.

Compound 1La can be converted in STEP 2 to compound 1Lb by deprotonation with a base such as LiHMDS in THF followed by treatment with acetic anhydride.

Compound 1Lc can be prepared in STEP 3 in a Suzuki coupling reaction between compounds 1Lb and R₆B(OR′)₂ or R₆BF₃K using for example [1,1′-bis(diphenylphosphino)ferrocene]dichloropalladium(II) (Pd(dppf)Cl₂), complex with DCM, as catalyst, in a mixture of toluene and water and in the presence of a base, for example cesium carbonate (Cs₂CO₃), by heating up to reflux of solvent.

Compound 1Lc can be converted in STEP 4 to compound 1M by hydrolysis with aqueous HCl solution by heating in methanol and DCM for example.

Herein is also provided a process for preparing a compound of formula (I) as defined above, wherein a compound of formula 1G:

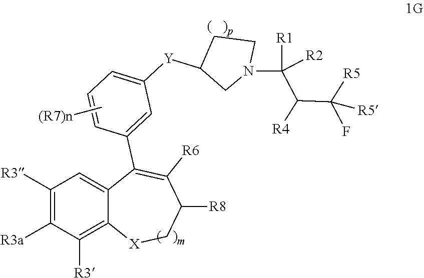

wherein R1, R2, R3′, R3″, R4, R5, R5′, R6, R7, R8, m, n, p, X and Y are as defined above and R3a is a hydrogen atom or carboxylic ester such as —COOMe, —COOEt, or -protected OH such as O-pivaloyl for example, is converted to compound of formula (I), in presence of a source of hydroxide ions, such as NaOH or LiOH in solution in methanol, said step being optionally preceded by a step for obtaining compound 1G, wherein a compound of formula 1F:

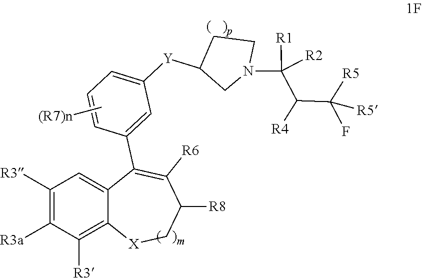

wherein, R1, R2, R3′, R3″, R4, R5, R5′, R7, R8, m, n, p, X and Y are as described above and R3a is as defined above,
is subjected to a Suzuki coupling with a boronic reagent R6B(OR′)₂, wherein —B(OR′)₂ is a boronic acid or a pinacolate ester and R6 is as defined above.

Herein is also provided a process for preparing a compound of formula (I) as described above, wherein a compound of formula 1Fa:

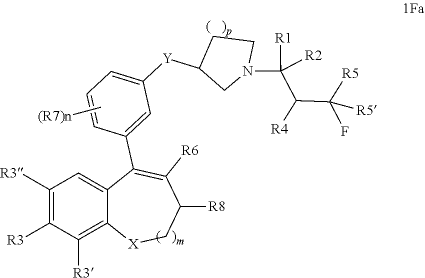

wherein R1, R2, R3, R3′, R3″, R4, R5, R5′, R7, R8, m, n, p, X and Y are as described above, is submitted to a Suzuki coupling with a boronic reagent R6B(OR′)₂, wherein —B(OR′)₂ is a boronic acid or a pinacolate ester and R6 is defined above, said step being optionally preceded by a step for obtaining compound 1Fa, wherein a compound of formula 1F:

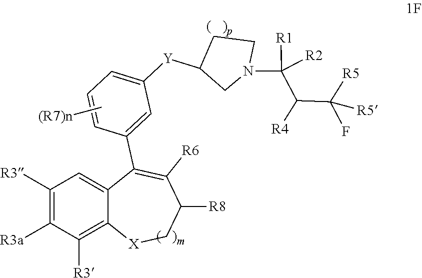

wherein R1, R2, R3′, R3″, R4, R5, R5′, R7, R8, m, n, p, X and Y are as described above and R3a is a hydrogen atom or a carboxylic ester such as —COOMe, —COOEt, or protected —OH such as O-pivaloyl, is converted to a compound 1Fa in the presence of a source of hydroxide ions, such as NaOH or LiOH in solution in methanol.

Herein are also provided the intermediates compounds selected from compounds of formula 1E, 1F, 1G and 1Fa, or any of its pharmaceutically acceptable salt:

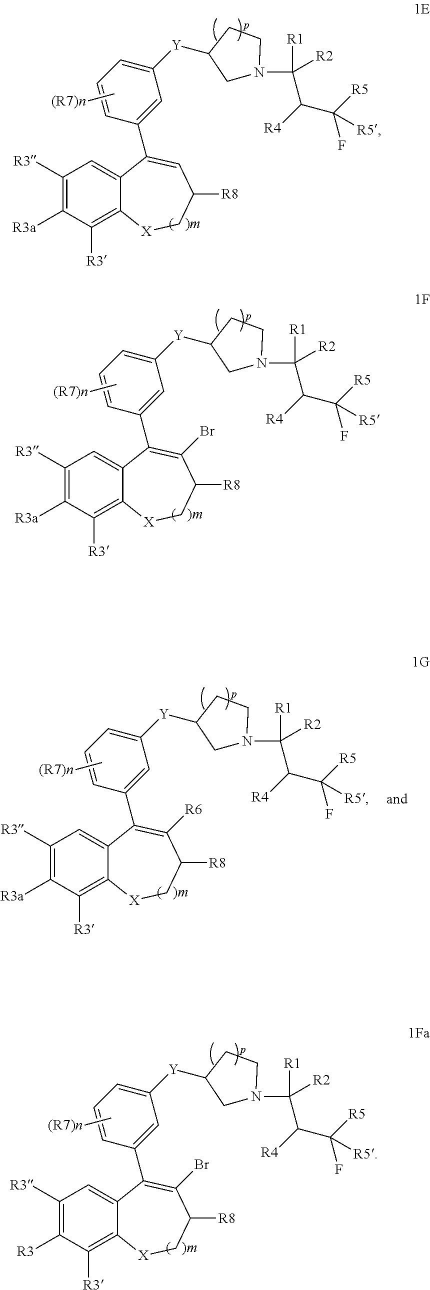

wherein R1, R2, R3, R3′, R3″, R4, R5, R5′, R6, R7, R8, m, n, p, X and Y are as defined above and R3a is a hydrogen atom or carboxylic ester such as —COOMe, —COOEt, or protected —OH such as 0-pivaloyl.

Herein is further provided the intermediate compound of formula 1D′, or any of its pharmaceutically acceptable salt:

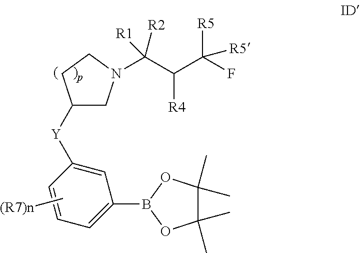

wherein R1, R2, R4, R5, R5′, R7, Y, p and n are as described above.

The present application also describes the intermediate compound of formula 1C′, or any of its pharmaceutically acceptable salt:

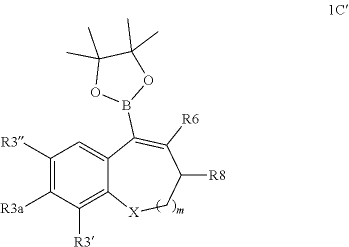

wherein R3a, R3′, R3″, X, m, R6 and R8 are as described above.

In another aspect, herein is also provided a process for the preparation of a compound of formula (I), wherein R3 is a —COOH group, comprising a deprotection step of a compound of formula 1G as defined above, optionally followed by a purification step.

Said purification step may for example consist, as illustrated in step 2 of example 1 herein after, in an acidification step, for example with an aqueous solution of hydrochloric acid.

The ¹H NMR Spectra at 400 and 500 MHz were performed on a Bruker Avance DRX-400 and Bruker Avance DPX-500 spectrometer, respectively, with the chemical shifts (δ in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

TABLE 1a

| Ex | Structure | Name |
|---|---|---|
| 1 | 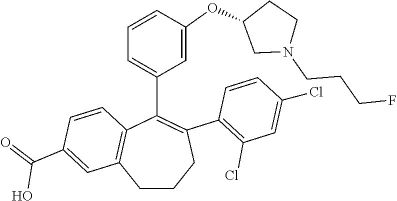 | (R)-8-(2,4-dichlorophenyl)-9-(3-((1-(3- fluoropropyl)pyrrolidin-3-yl)oxy)phenyl)- 6,7-dihydro-5H-benzo[7]annulene-3- carboxylic acid |
| 1a Comparative example | 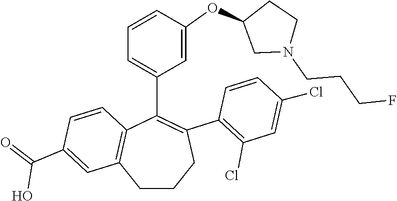 | (S)-8-(2,4-dichlorophenyl)-9-(3-((1-(3- fluoropropyl)pyrrolidin-3-yl)oxy)phenyl)- 6,7-dihydro-5H-benzo[7]annulene-3- carboxylic acid |
| 2 | 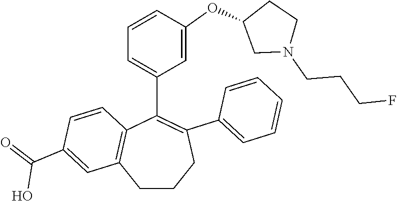 | (R)-9-(3-((1-(3-fluoropropyl)pyrrolidin-3- yl)oxy)phenyl)-8-phenyl-6,7-dihydro-5H- benzo[7]annulene-3-carboxylic acid |
| 2a Comparative example | 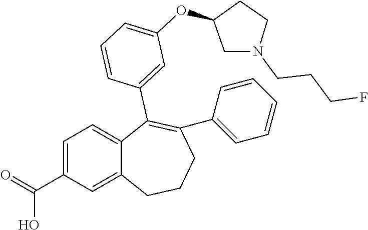 [SMILES: O=C(O)c1ccc2c(c1)CCCC(c1ccccc1)=C2c1cccc(O[C@H]2CCN(CCCF)C2)c1] | (S)-9-(3-((1-(3-fluoropropyl)pyrrolidin-3- yl)oxy)phenyl)-8-phenyl-6,7-dihydro-5H- benzo[7]annulene-3-carboxylic acid |
| 3 | 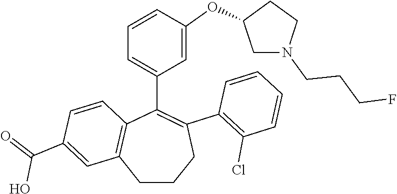 | (R)-8-(2-chlorophenyl)-9-(3-((1-(3- fluoropropyl)pyrrolidin-3-yl)oxy)phenyl)- 6,7-dihydro-5H-benzo[7]annulene-3- carboxylic acid |
| 4 | 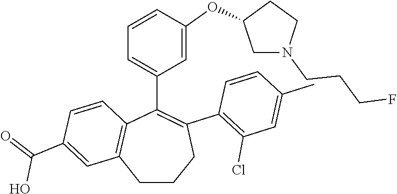 | (R)-8-(2-chloro-4-methylphenyl)-9-(3-((1- (3-fluoropropyl)pyrrolidin-3- yl)oxy)phenyl)-6,7-dihydro-5H- benzo[7]annulene-3-carboxylic acid |
| 5 | 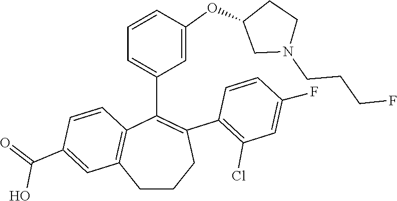 [SMILES: O=C(O)c1ccc2c(c1)CCCC(c1ccc(F)cc1Cl)=C2c1cccc(O[C@@H]2CCN(CCCF)C2)c1] | (R)-8-(2-chloro-4-fluorophenyl)-9-(3-((1- (3-fluoropropyl)pyrrolidin-3- yl)oxy)phenyl)-6,7-dihydro-5H- benzo[7]annulene-3-carboxylic acid |
| 6 | 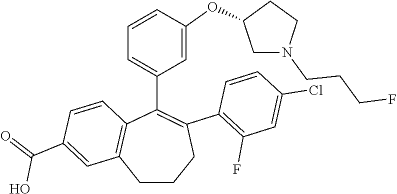 | (R)-8-(4-chloro-2-fluorophenyl)-9-(3-((1- (3-fluoropropyl)pyrrolidin-3- yl)oxy)phenyl)-6,7-dihydro-5H- benzo[7]annulene-3-carboxylic acid |
| 7 | 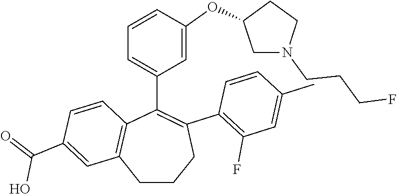 | (R)-8-(2-fluoro-4-methylphenyl)-9-(3-((1- (3-fluoropropyl)pyrrolidin-3- yl)oxy)phenyl)-6,7-dihydro-5H- benzo[7]annulene-3-carboxylic acid |
| 8 | 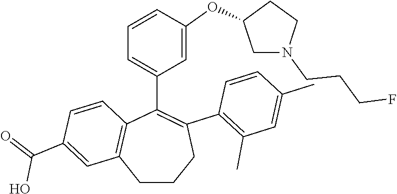 | (R)-8-(2,4-dimethylphenyl)-9-(3-((1-(3- fluoropropyl)pyrrolidin-3-yl)oxy)phenyl)- 6,7-dihydro-5H-benzo[7]annulene-3- carboxylic acid |
| 9 | 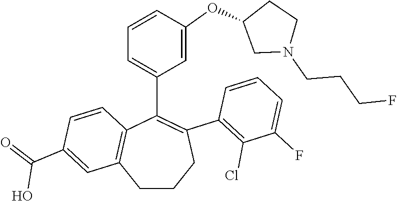 | (R)-8-(2-chloro-3-fluorophenyl)-9-(3-((1- (3-fluoropropyl)pyrrolidin-3- yl)oxy)phenyl)-6,7-dihydro-5H- benzo[7]annulene-3-carboxylic acid |
| 10 | 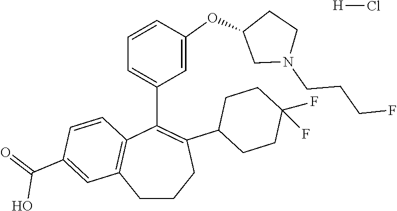 | (R)-8-(4,4-difluorocyclohexyl)-9-(3-((1-(3- fluoropropyl)pyrrolidin-3-yl)oxy)phenyl)- 6,7-dihydro-5H-benzo[7]annulene-3- carboxylic acid hydrochloride |
| 11 | 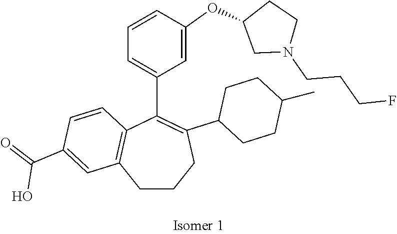 | (R)-9-(3-((1-(3-fluoropropyl)pyrrolidin-3- yl)oxy)phenyl)-8-(4-methylcyclohexyl)-6,7- dihydro-5H-benzo[7]annulene-3-carboxylic acid, Isomer 1 |
| 12 | 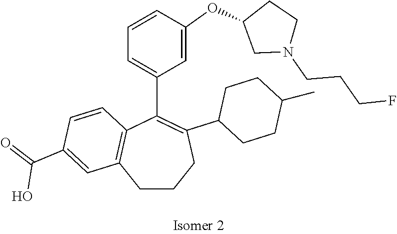 | (R)-9-(3-((1-(3-fluoropropyl)pyrrolidin-3- yl)oxy)phenyl)-8-(4-methylcyclohexyl)-6,7- dihydro-5H-benzo[7]annulene-3-carboxylic acid, Isomer 2 |
| 13 | 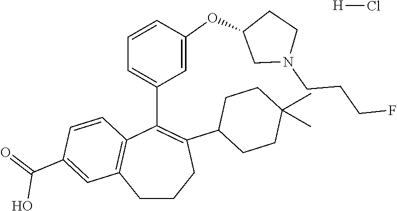 | (R)-8-(4,4-dimethylcyclohexyl)-9-(3-((1-(3- fluoropropyl)pyrrolidin-3-yl)oxy)phenyl)- 6,7-dihydro-5H-benzo[7]annulene-3- carboxylic acid, hydrochloride |
| 14 | 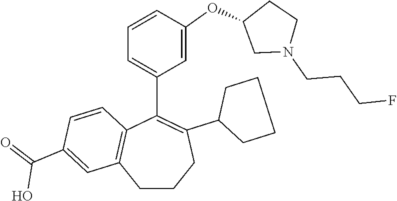 | (R)-8-cyclopentyl-9-(3-((1-(3- fluoropropyl)pyrrolidin-3-yl)oxy)phenyl)- 6,7-dihydro-5H-benzo[7]annulene-3- carboxylic acid |
| 15 | 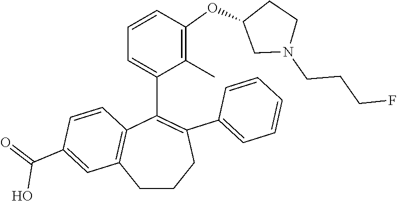 | (R)-9-(3-((1-(3-fluoropropyl)pyrrolidin-3- yl)oxy)-2-methylphenyl)-8-phenyl-6,7- dihydro-5H-benzo[7]annulene-3-carboxylic acid |
| 16 | 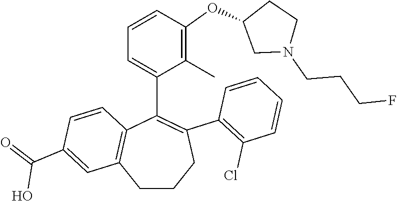 | (R)-8-(2-chlorophenyl)-9-(3-((1-(3- fluoropropyl)pyrrolidin-3-yl)oxy)-2- methylphenyl)-6,7-dihydro-5H- benzo[7]annulene-3-carboxylic acid |
| 17 | 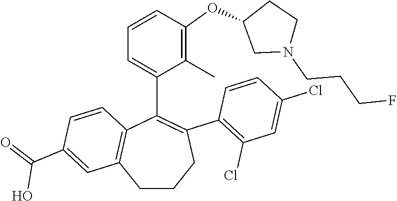 | (R)-8-(2,4-dichlorophenyl)-9-(3-((1-(3- fluoropropyl)pyrrolidin-3-yl)oxy)-2- methylphenyl)-6,7-dihydro-5H- benzo[7]annulene-3-carboxylic acid |
| 18 | 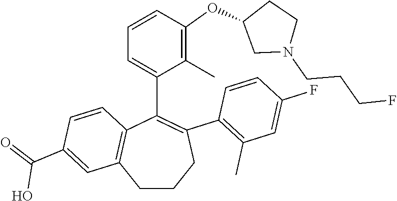 | (R)-8-(4-fluoro-2-methylphenyl)-9-(3-((1- (3-fluoropropyl)pyrrolidin-3-yl)oxy)-2- methylphenyl)-6,7-dihydro-5H- benzo[7]annulene-3-carboxylic acid |
| 19 | 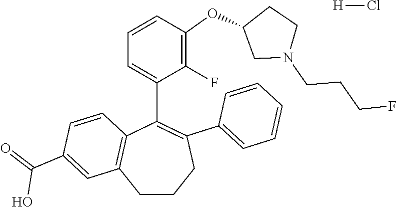 | (R)-9-(2-fluoro-3-((1-(3- fluoropropyl)pyrrolidin-3-yl)oxy)phenyl)-8- phenyl-6,7-dihydro-5H-benzo[7]annulene- 3-carboxylic acid, hydrochloride |
| 20 | 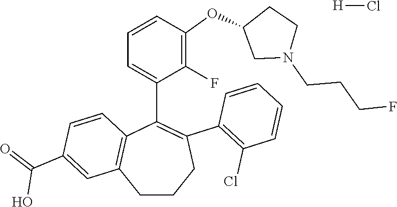 | (R)-8-(2-chlorophenyl)-9-(2-fluoro-3-((1- (3-fluoropropyl)pyrrolidin-3- yl)oxy)phenyl)-6,7-dihydro-5H- benzo[7]annulene-3-carboxylic acid, hydrochloride |
| 21 | 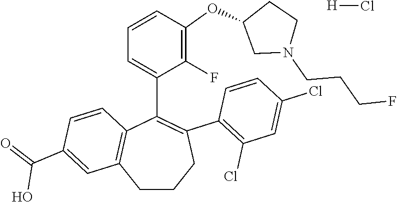 | (R)-8-(2,4-dichlorophenyl)-9-(2-fluoro-3- ((1-(3-fluoropropyl)pyrrolidin-3- yl)oxy)phenyl)-6,7-dihydro-5H- benzo[7]annulene-3-carboxylic acid, hydrochloride |
| 22 | 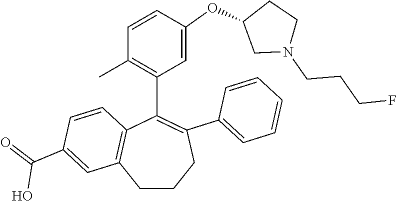 | (R)-9-(5-((1-(3-fluoropropyl)pyrrolidin-3- yl)oxy)-2-methylphenyl)-8-phenyl-6,7- dihydro-5H-benzo[7]annulene-3-carboxylic acid |
| 23 | 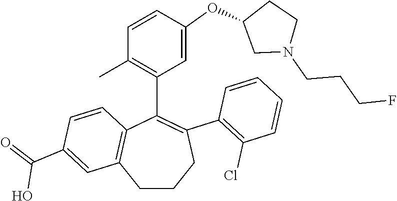 | (R)-8-(2-chlorophenyl)-9-(5-((1-(3- fluoropropyl)pyrrolidin-3-yl)oxy)-2- methylphenyl)-6,7-dihydro-5H- benzo[7]annulene-3-carboxylic acid |
| 24 | 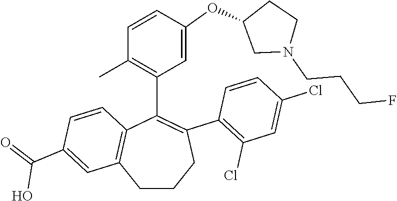 | (R)-8-(2-chlorophenyl)-9-(5-((1-(3- fluoropropyl)pyrrolidin-3-yl)oxy)-2- methylphenyl)-6,7-dihydro-5H- benzo[7]annulene-3-carboxylic acid |
| 25 | 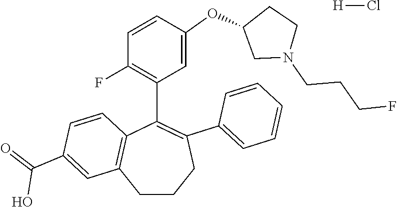 | (R)-9-(2-fluoro-5-((1-(3- fluoropropyl)pyrrolidin-3-yl)oxy)phenyl)-8- phenyl-6,7-dihydro-5H-benzo[7]annulene- 3-carboxylic acid, hydrochloride |
| 26 | 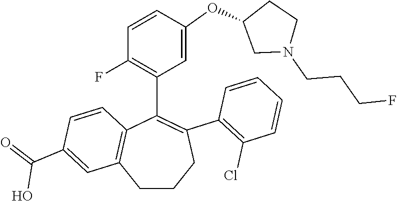 | (R)-8-(2-chlorophenyl)-9-(2-fluoro-5-((1- (3-fluoropropyl)pyrrolidin-3- yl)oxy)phenyl)-6,7-dihydro-5H- benzo[7]annulene-3-carboxylic acid, hhdrochloride |
| 27 | 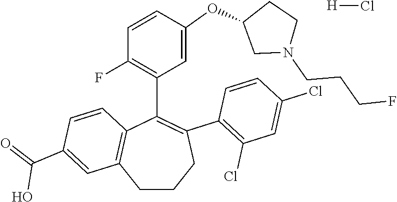 | (R)-8-(2,4-dichlorophenyl)-9-(2-fluoro-5- ((1-(3-fluoropropyl)pyrrolidin-3- yl)oxy)phenyl)-6,7-dihydro-5H- benzo[7]annulene-3-carboxylic acid hydrochloride |
| 28 | 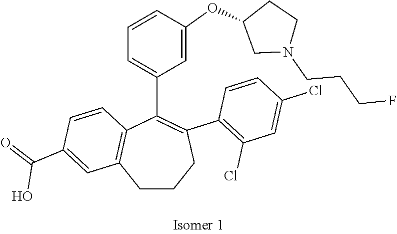 | 8-(2,4-dichlorophenyl)-9-(3-((1-(3- fluoropropyl)pyrrolidin-3- yl)methyl)phenyl)-6,7-dihydro-5H- benzo[7]annulene-3-carboxylic acid, Isomer 1 |
| 29 | 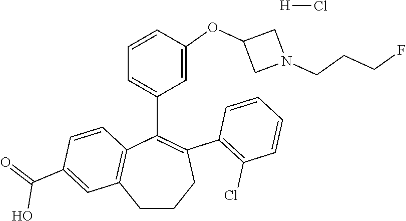 | 8-(2-chlorophenyl)-9-(3-((1-(3- fluoropropyl)azetidin-3-yl)oxy)phenyl)-6,7- dihydro-5H-benzo[7]annulene-3-carboxylic acid hydrochloride |
| 30 | 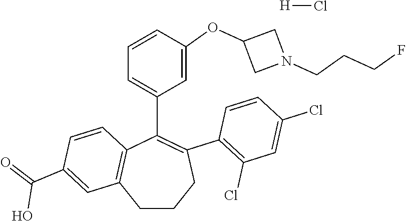 | 8-(2,4-dichlorophenyl)-9-(3-((1-(3- fluoropropyl)azetidin-3-yl)oxy)phenyl)-6,7- dihydro-5H-benzo[7]annulene-3-carboxylic acid hydrochloride |
| 31 | 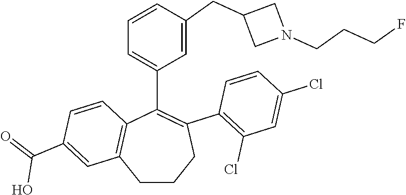 [SMILES: O=C(O)c1ccc2c(c1)CCCC(c1ccc(Cl)cc1Cl)=C2c1cccc(CC2CN(CCCF)C2)c1] | 8-(2,4-dichlorophenyl)-9-(3-((1-(3- fluoropropyl)azetidin-3-yl)methyl)phenyl)- 6,7-dihydro-5H-benzo[7]annulene-3- carboxylic acid |
| 32 | 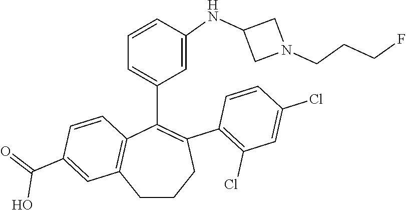 [SMILES: O=C(O)c1ccc2c(c1)CCCC(c1ccc(Cl)cc1Cl)=C2c1cccc(NC2CN(CCCF)C2)c1] | 8-(2,4-dichlorophenyl)-9-(3-((1-(3- fluoropropyl)azetidin-3-yl)amino)phenyl)- 6,7-dihydro-5H-benzo[7]annulene-3- carboxylic acid |
| 33 | 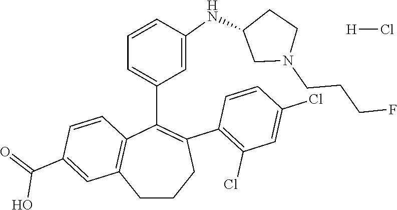 [SMILES: O=C(O)c1ccc2c(c1)CCCC(c1ccc(Cl)cc1Cl)=C2c1cccc(N[C@@H]2CCN(CCCF)C2)c1 ; Cl] | (R)-8-(2,4-dichlorophenyl)-9-(3-((1-(3- fluoropropyl)pyrrolidin-3- yl)amino)phenyl)-6,7-dihydro-5H- benzo[7]annulene-3-carboxylic acid hydrochloride |
| 34 | 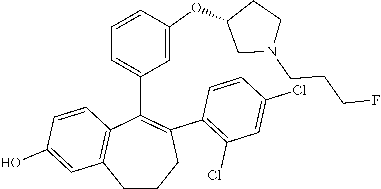 | (R)-8-(2,4-dichlorophenyl)-9-(3-((1-(3- fluoropropyl)pyrrolidin-3-yl)oxy)phenyl)- 6,7-dihydro-5H-benzo[7]annulen-3-ol |
| 35 | 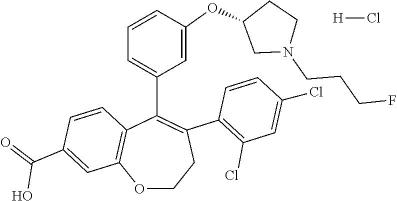 | (R)-4-(2,4-dichlorophenyl)-5-(3-((1-(3- fluoropropyl)pyrrolidin-3-yl)oxy)phenyl)- 2,3-dihydrobenzo[b]oxepine-8-carboxylic acid hydrochloride |
| 36 | 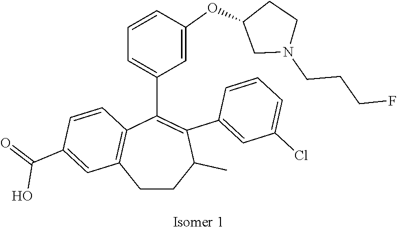 | 8-(3-chlorophenyl)-9-(3-(((R)-1-(3- fluoropropyl)pyrrolidin-3-yl)oxy)phenyl)-7- methyl-6,7-dihydro-5H-benzo[7]annulene- 3-carboxylic acid, Isomer 1 |
| 37 | 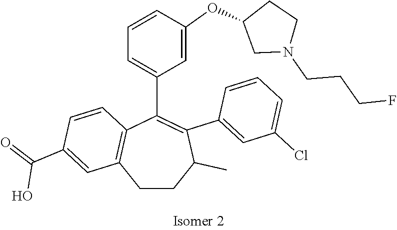 | 8-(3-chlorophenyl)-9-(3-(((R)-1-(3- fluoropropyl)pyrrolidin-3-yl)oxy)phenyl)-7- methyl-6,7-dihydro-5H-benzo[7]annulene- 3-carboxylic acid, Isomer 2 |
| 38 | 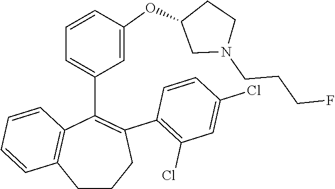 | (R)-3-(3-(8-(2,4-dichlorophenyl)-6,7- dihydro-5H-benzo[7]annulen-9- yl)phenoxy)-1-(3-fluoropropyl)pyrrolidine |
| 39 | 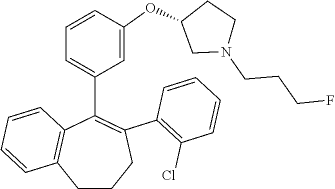 | (R)-3-(3-(8-(2-chlorophenyl)-6,7-dihydro- 5H-benzo[7]annulen-9-yl)phenoxy)-1-(3- fluoropropyl)pyrrolidine |
| 40 | 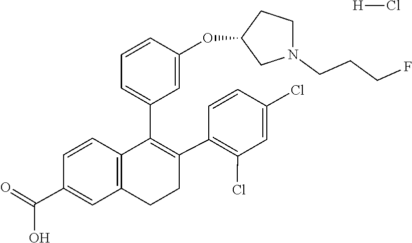 [SMILES: Cl ; O=C(O)c1ccc2c(c1)CCC(c1ccc(Cl)cc1Cl)=C2c1cccc(O[C@@H]2CCN(CCCF)C2)c1] | (R)-6-(2,4-dichlorophenyl)-5-(3-((1-(3- fluoropropyl)pyrrolidin-3-yl)oxy)phenyl)- 7,8-dihydronaphthalene-2-carboxylic acid hydrochloride |
| 41 | 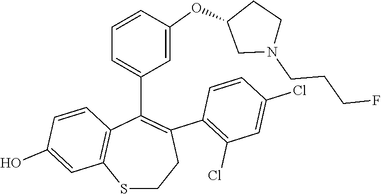 | (R)-4-(2,4-dichlorophenyl)-5-(3-((1-(3- fluoropropyl)pyrrolidin-3-yl)oxy)phenyl)- 2,3-dihydrobenzo[b]thiepin-8-ol |

(the first column ″Ex″ corresponds to the compound and example number)

TABLE 1b

| | | | MASS: |
|---|---|---|---|
| | | | LC/MS |
| | Preparation | | (m/z, |
| Ex | Method | NMR | MH+): |
| 1 | A | 1H NMR (400 MHz, DMSO-d6) δ ppm 1.45-1.69 | 554 |
| | | (m, 1 H), 1.70-1.87 (m, 2 H), 1.98-2.27 (m, 5 H), | |
| | | 2.28-2.39 (m, 2 H), 2.41-2.47 (m, 2 H), 2.56-2.65 | |
| | | (m, 2 H), 2.87-3.00 (m, 2 H), 4.47 (dt, J = 47, 6 Hz, 2 | |
| | | H), 4.58 (td, J = 6, 3 Hz, 1 H), 6.32-6.37 (m, 1 H), | |
| | | 6.41 (d, J = 8 Hz, 1 H), 6.65 (dd, J = 8, 2 Hz, 1 H), 6.89 | |
| | | (d, J = 8 Hz, 1 H), 7.06 (t, J = 8 Hz, 1 H), 7.18-7.31 (m, | |
| | | 2 H), 7.60 (d, J = 2 Hz, 1 H), 7.75 (dd, J = 8, 2 Hz, 1 H), | |
| | | 7.92 (d, J = 2 Hz, 1 H), 11.49-14.29 (m, 1 H) | |
| 1a | A | 1H NMR (400 MHz, DMSO-d6) δ ppm 1.62-2.53 | 554 |
| | | (m, 4 H), 2.11-2.25 (m, 4 H), 2.83-4.09 (m partially | |
| | | hidden, 6 H), 2.91-3.01 (m, 2 H), 4.51 (dt, J = 47.0, | |
| | | 5.4 Hz, 2 H), 4.73-5.14 (m, 1 H), 6.42 (br s, 1 H), | |
| | | 6.50 (br d, J = 6.7 Hz, 1 H), 6.74 (br d, J = 6.7 Hz, 1 H), | |
| | | 6.89 (d, J = 8.0 Hz, 1 H), 7.11 (br t, J = 7.9 Hz, 1 H), | |
| | | 7.22-7.27 (m, 1 H), 7.26-7.34 (m, 1 H), 7.61 (d, | |
| | | J = 1.8 Hz, 1 H), 7.76 (dd, J = 8.0, 1.8 Hz, 1 H), 7.93 (d, | |
| | | J = 1.7 Hz, 1 H), 9.96-10.58 (m, 1 H), 12.89 (br s, 1 | |
| | | H) | |
| 2 | A | 1H NMR (400 MHz, DMSO-d6) δ ppm 1.53 (m, 1 H), | 486 |
| | | 1.66-1.85 (m, 2 H), 1.98 (m, 1 H), 2.08-2.20 (m, 2 | |
| | | H), 2.21-2.37 (m, 4 H), 2.42 (t, J = 7 Hz, 2 H), 2.54- | |
| | | 2.64 (m, 1 H), 2.86 (t, J = 7 Hz, 2 H), 4.46 (dt, J = 48, 5 | |
| | | Hz, 2 H), 4.52-4.52 (m, 1 H), 4.52-4.60 (m, 1 H), | |
| | | 6.31-6.36 (m, 1 H), 6.41 (d, J = 8 Hz, 1 H), 6.62 (dd, | |
| | | J = 8, 2 Hz, 1 H), 6.88 (d, J = 8 Hz, 1 H), 7.04 (t, J = 8 Hz, | |
| | | 1 H), 7.10-7.27 (m, 5 H), 7.74 (dd, J = 8, 2 Hz, 1 H), | |
| | | 7.90 (d, J = 2 Hz, 1 H), 9.97-14.06 (m, 1 H) | |
| 2a | A | 1H NMR (400 MHz, DMSO-d6) δ ppm 1.59-2.45 (m, | 486 |
| | | 4 H), 2.09-2.19 (m, 2 H), 2.25-2.32 (m, 2 H), 2.69- | |
| | | 3.99 (m partially hidden, 6 H), 2.87 (br t, J = 6.8 Hz, 2 | |
| | | H), 4.51 (dt, J = 47.0, 6.0 Hz, 2 H), 4.70-5.05 (m, 1 H), | |
| | | 6.42 (br s, 1 H), 6.49 (br d, J = 7.3 Hz, 1 H), 6.71 (br d, | |
| | | J = 7.3 Hz, 1 H), 6.88 (d, J = 8.0 Hz, 1 H), 7.09 (br t, | |
| | | J = 7.3 Hz, 1 H), 7.12-7.27 (m, 5 H), 7.75 (dd, J = 8.1, | |
| | | 1.8 Hz, 1 H), 7.91 (d, J = 1.7 Hz, 1 H), 9.87-10.62 (m, | |
| | | 1 H), 12.48-13.26 (m, 1 H) | |
| 3 | A | 1H NMR (400 MHz, DMSO-d6) δ ppm 1.42-1.69 (m, | 520 |
| | | 1 H), 1.70-1.87 (m, 2 H), 2.11-2.28 (m, 4 H), 2.29- | |
| | | 2.38 (m, 2 H), 2.39-2.47 (m, 3 H), 2.55-2.75 (m, 2 | |
| | | H), 2.87-3.00 (m, 2 H), 4.31-4.58 (m, 3 H), 6.36 (s, | |
| | | 1 H), 6.41 (d, J = 8 Hz, 1 H), 6.61 (dd, J = 8, 2 Hz, 1 H), | |
| | | 6.88 (d, J = 8 Hz, 1 H), 7.02 (t, J = 8 Hz, 1 H), 7.12-7.28 | |
| | | (m, 3 H), 7.43 (d, J = 7 Hz, 1 H), 7.75 (dd, J = 8, 2 Hz, 1 | |
| | | H), 7.91 (d, J = 2 Hz, 1 H) | |
| 4 | A | 1H NMR (400 MHz, DMSO-d6) δ ppm 1.66-2.23 (m, | 534 |
| | | 8 H), 2.25 (s, 3 H), 2.89-2.99 (m, 2 H), 3.02-3.30 | |
| | | (m, 4 H), 3.45-3.94 (m, 2 H), 4.53 (dt, J = 47, 6 Hz, 2 | |
| | | H), 4.75-4.98 (m, 1 H), 6.43 (s, 1 H), 6.51 (d, J = 7 Hz, | |
| | | 1 H), 6.68-6.78 (m, 1 H), 6.88 (d, J-8 Hz, 1 H), 6.98- | |
| | | 7.03 (m, 1 H), 7.04-7.14 (m, 2 H), 7.27 (s, 1 H), 7.76 | |
| | | (dd, J = 8, 2 Hz, 1 H), 7.92 (d, J = 2 Hz, 1 H), 9.83- | |
| | | 11.28 (m, 1 H), 12.91 (br s, 1 H) | |
| 5 | A | 1H NMR (400 MHz, DMSO-d6) δ ppm 1.72-2.57 (m | 538 |
| | | partially hidden, 8 H), 2.96 (m, 2 H), 3.05-3.27 (m, 4 | |
| | | H), 3.46-4.04 (m, 2 H), 4.52 (dt, J = 47, 6 Hz, 2 H), | |
| | | 4.80-5.10 (m, 1 H), 6.43 (s, 1 H), 6.51 (d, J = 7 Hz, 1 | |
| | | H), 6.74 (d, J = 8 Hz, 1 H), 6.89 (d, J = 8 Hz, 1 H), 7.04- | |
| | | 7.16 (m, 2 H), 7.27 (dd, J = 9, 6 Hz, 1 H), 7.44 (dd, J = 9, | |
| | | 3 Hz, 1 H), 7.76 (dd, J = 8, 2 Hz, 1 H), 7.93 (d, J = 2 Hz, | |
| | | 1 H), 9.64-11.34 (m, 1 H), 12.92 (br s, 1 H) | |
| 6 | A | 1H NMR (400 MHz, DMSO-d6) δ ppm 1.68-2.45 (m, | 538 |
| | | 8 H), 2.88 (t, J = 6 Hz, 2 H), 3.03-3.30 (m, 4 H), 3.43- | |
| | | 4.03 (m, 2 H), 4.53 (dt, J = 47, 6 Hz, 2 H), 4.82-5.18 | |
| | | (m, 1 H), 6.43 (br s, 1 H), 6.51 (d, J = 8 Hz, 1 H), 6.77 | |
| | | (d, J = 7 Hz, 1 H), 6.89 (d, J = 8 Hz, 1 H), 7.13 (t, J = 8 | |
| | | Hz, 1 H), 7.18 (dd, J = 8, 2 Hz, 1 H), 7.28 (t, J = 8 Hz, 1 | |
| | | H), 7.34 (dd, J = 10, 2 Hz, 1 H), 7.77 (dd, J = 8, 2 Hz, 1 | |
| | | H), 7.93 (d, J = 2 Hz, 1 H), 9.78-10.98 (m, 1 H), 12.93 | |
| | | (br s, 1 H) | |
| 7 | A | 1H NMR (400 MHz, DMSO-d6) δ ppm 1.52-1.66 (m, | 518 |
| | | 1 H), 1.71-1.93 (m, 2 H), 1.98-2.09 (m, 1 H), 2.09- | |
| | | 2.22 (m, 4 H), 2.25 (s, 3 H), 2.29-2.82 (m hidden, 6 | |
| | | H), 2.87 (t, J = 7 Hz, 2 H), 4.47 (dt, J = 47, 6 Hz, 2 H), | |
| | | 4.61 (m, 1 H), 6.36 (s, 1 H), 6.41 (br d, J = 8 Hz, 1 H), | |
| | | 6.65 (br d, J = 9 Hz, 1 H), 6.83-6.90 (m, 2 H), 6.92 (d, | |
| | | J = 11 Hz, 1 H), 7.06 (dt, J = 11, 8 Hz, 2 H), 7.75 (dd, | |
| | | J = 8, 2 Hz, 1 H), 7.91 (d, J = 2 Hz, 1 H), 12.88 (br s, 1 | |
| | | H) | |
| 8 | A | 1H NMR (400 MHz, DMSO-d6) δ ppm 2.14 (s, 11 H), | 514 |
| | | 2.21 (s, 3 H), 2.79-2.96 (m, 2 H), 2.98-3.31 (m, 4 | |
| | | H), 3.39-3.87 (m, 2 H), 4.53 (dt, J = 47, 6 Hz, 2 H), | |
| | | 4.73-4.92 (m, 1 H), 6.36 (br s, 1 H), 6.44 (d, J = 8 Hz, | |
| | | 1 H), 6.69 (d, J = 7 Hz, 1 H), 6.84-6.92 (m, 2 H), 6.93- | |
| | | 7.00 (m, 2 H), 7.06 (t, J = 8 Hz, 1 H), 7.75 (dd, J = 8, 2 | |
| | | Hz, 1 H), 7.92 (d, J = 2 Hz, 1 H), 9.67-11.12 (m, 1 H), | |
| | | 12.88 (s, 1 H) | |
| 9 | A | 1H NMR (400 MHz, DMSO-d6) δ ppm 1.64-2.31 (m, | 538 |
| | | 8 H), 2.39 (m, 1 H), 2.97 (t, J = 4 Hz, 2 H), 3.05-3.30 | |
| | | (m, 3 H), 3.44-3.98 (m, 2 H), 4.53 (dt, J = 47, 6 Hz, 2 | |
| | | H), 4.93 (m, 1 H), 6.43 (br s, 1 H), 6.52 (d, J = 8 Hz, 1 | |
| | | H), 6.75 (d, J = 8 Hz, 1 H), 6.90 (d, J = 8 Hz, 1 H), 7.02- | |
| | | 7.16 (m, 2 H), 7.19-7.38 (m, 2 H), 7.77 (dd, J = 8, 2 | |
| | | Hz, 1 H), 7.94 (d, J = 2 Hz, 1 H), 10.08-10.75 (m, 1 | |
| | | H), 12.94 (br s, 1 H) | |
| 10 | B | 1H NMR (400 MHz, DMSO-d6) δ ppm 1.52-1.78 (m, | 528 |
| | | 6 H), 1.80-1.92 (m, 2 H), 1.93-2.29 (m, 6 H), 2.36- | |
| | | 3.53 (m hidden, 6 H), 2.74 (t, J = 7 Hz, 2 H), 3.55-3.85 | |
| | | (m, 2 H), 4.53 (dt, J = 47, 6 Hz, 3 H), 5.07-5.22 (m, 1 | |
| | | H), 6.62 (m, 1 H), 6.74 (br d, J = 7 Hz, 1 H), 6.79 (d, | |
| | | J = 8 Hz, 1 H), 6.92 (br d, J = 8 Hz, 1 H), 7.33 (t, J = 8 Hz, | |
| | | 1 H), 7.68 (dd, J = 8, 2 Hz, 1 H), 7.84 (d, J = 2 Hz, 1 H), | |
| | | 9.79-11.05 (m, 1 H), 12.82 (s, 1 H) | |
| 11 | B | 1H NMR (400 MHz, DMSO-d6) δ ppm 0.99 (d, J = 7 | 506 |
| | | Hz, 3 H), 1.30-1.41 (m, 3 H), 1.42-1.57 (m, 2 H), | |
| | | 1.57-1.75 (m, 2 H), 1.80-1.97 (m, 3 H), 1.99-2.28 | |
| | | (m, 5 H), 2.33-2.41 (m, 1 H), 2.74 (t, J = 7 Hz, 2 H), | |
| | | 2.98-3.52 (m partially hidden, 6 H), 3.53-4.04 (m, 2 | |
| | | H), 4.54 (dt, J = 47, 6 Hz, 2 H), 5.09-5.23 (m, 1 H), | |
| | | 6.61 (br s, 1 H), 6.73 (br d, J = 7 Hz, 1 H), 6.80 (d, J = 8 | |
| | | Hz, 1 H), 6.91 (br dd, J = 8, 2 Hz, 1 H), 7.32 (t, J = 8 Hz, | |
| | | 1 H), 7.68 (dd, J = 8, 2 Hz, 1 H), 7.84 (d, J = 2 Hz, 1 H), | |
| | | 9.55-11.55 (m, 1 H), 12.80 (br s, 1 H) | |
| 12 | B | 1H NMR (400 MHz, DMSO-d6) δ ppm 0.48-1.00 (m, | 506 |
| | | 5 H), 1.20-1.40 (m, 1 H), 1.41-1.55 (m, 2 H), 1.56- | |
| | | 1.78 (m, 4 H), 1.81-1.97 (m, 2 H), 1.98-2.26 (m, 6 | |
| | | H), 2.31-2.38 (m, 1 H), 2.73 (t, J = 7 Hz, 2 H), 3.08- | |
| | | 3.49 (m partially hidden, 4 H), 3.52-4.15 (m, 2 H), | |
| | | 4.54 (dt, J = 47, 6 Hz, 2 H), 5.07-5.24 (m, 1 H), 6.61 | |
| | | (br s, 1 H), 6.72 (br d, J = 8 Hz, 1 H), 6.79 (d, J = 8 Hz, 1 | |
| | | H), 6.91 (dd, J = 8, 2 Hz, 1 H), 7.32 (t, J = 8 Hz, 1 H), | |
| | | 7.67 (dd, J = 8, 2 Hz, 1 H), 7.83 (d, J = 2 Hz, 1 H), 9.72- | |
| | | 11.38 (m, 1 H), 12.80 (br s, 1 H) | |
| 13 | B | 1H NMR (400 MHz, DMSO-d6) δ ppm 0.84 (s, 3 H), | 520 |
| | | 0.91 (s, 3 H), 0.95-1.08 (m, 2 H), 1.31-1.48 (m, 4 | |
| | | H), 1.53-1.70 (m, 2 H), 1.84-1.95 (m, 2 H), 1.96- | |
| | | 2.32 (m, 7 H), 2.73 (t, J = 7 Hz, 2 H), 3.01-3.44 (m | |
| | | partially hidden, 4 H), 3.47-4.05 (m, 2 H), 4.53 (dt, | |
| | | J = 47, 6 Hz, 2 H), 5.07-5.23 (m, 1 H), 6.60 (br s, 1 H), | |
| | | 6.72 (br d, J = 7 Hz, 1 H), 6.79 (d, J = 8 Hz, 1 H), 6.90 | |
| | | (dd, J = 8, 2 Hz, 1 H), 7.31 (t, J = 8 Hz, 1 H), 7.67 (dd, | |
| | | J = 8, 2 Hz, 1 H), 7.83 (d, J = 2 Hz, 1 H), 10.10-10.99 | |
| | | (m, 1 H), 12.80 (br s, 1 H) | |
| 14 | B | 1H NMR (400 MHz, DMSO-d6) δ ppm 1.39-1.56 (m, | 478 |
| | | 4 H), 1.58-1.71 (m, 4 H), 1.71-1.91 (m, 5 H), 2.06- | |
| | | 2.27 (m partially hidden, 4 H), 2.29-2.64 (m, 4 H), | |
| | | 2.70-2.82 (m, 4 H), 4.47 (dt, J = 47, 6 Hz, 2 H), 4.77- | |
| | | 4.89 (m, 1 H), 6.53 (s, 1 H), 6.61 (d, J = 8 Hz, 1 H), | |
| | | 6.76 (d, J = 8 Hz, 1 H), 6.79 (dd, J = 8, 2 Hz, 1 H), 7.24 | |
| | | (t, J = 8 Hz, 1 H), 7.65 (dd, J = 8, 2 Hz, 1 H), 7.82 (d, J = 2 | |
| | | Hz, 1 H), 11.83-13.43 (m, 1 H) | |
| 15 | A | 1H NMR (400 MHz, DMSO-d6) δ ppm 1.62-1.89 (m, | 500 |
| | | 7 H), 2.12-2.32 (m, 6 H), 2.35-2.48 (m, 2 H), 2.63 | |
| | | (m, 1 H), 2.79 (m, 1 H), 2.86-2.96 (m, 2 H), 4.33- | |
| | | 4.58 (m, 2 H), 4.75 (m, 1 H), 6.49 (d, J = 8 Hz, 1 H), | |
| | | 6.67 (dd, J = 8, 4 Hz, 1 H), 6.73 (dd, J = 8, 1 Hz, 1 H), | |
| | | 6.91 (t, J = 8 Hz, 1 H), 7.05-7.21 (m, 5 H), 7.70 (dd, | |
| | | J = 8, 2 Hz, 1 H), 7.90 (d, J = 2 Hz, 1 H), 11.68-13.73 | |
| | | (m, 1 H) | |
| 16 | A | 1H NMR (400 MHz, DMSO-d6) δ ppm 1.58-1.93 (m, | 534 |
| | | 4 H), 2.08-2.31 (m, 4 H), 2.35-3.13 (m partially | |
| | | hidden, 11 H), 4.32-4.59 (m, 2 H), 4.67-4.79 (m, 1 | |
| | | H), 6.65 (br d, J = 8 Hz, 1 H), 6.76 (d, J = 8 Hz, 1 H), | |
| | | 6.90 (t, J = 8 Hz, 1 H), 7.00-7.23 (m, 3 H), 7.25-7.36 | |
| | | (m, 1 H), 7.40 (br d, J = 8 Hz, 1 H), 7.72 (dd, J = 8, 2 Hz, | |
| | | 1 H), 7.91 (d, J = 1 Hz, 1 H) | |
| 17 | A | 1H NMR (400 MHz, DMSO-d6) δ ppm 1.61-1.92 (m, | 568 |
| | | 7 H), 2.14-2.31 (m, 6 H), 2.36-2.66 (m partially | |
| | | hidden, 3 H), 2.78 (m, 1 H), 2.87-3.09 (m, 2 H), 4.30- | |
| | | 4.60 (m, 2 H), 4.66-4.83 (m, 1 H), 6.49-6.70 (m, 2 | |
| | | H), 6.75 (d, J = 8 Hz, 1 H), 6.85-6.97 (m, 1 H), 6.98- | |
| | | 7.63 (m, 3 H), 7.65-7.78 (m, 1 H), 7.91 (s, 1 H), | |
| | | 11.24-14.12 (m, 1 H) | |
| 18 | A | 1H NMR (400 MHz, DMSO-d6) δ ppm 1.59-1.88 (m, | 532 |
| | | 6 H), 2.04-2.36 (m, 8 H), 2.36-2.47 (m, 4 H), 2.62 | |
| | | (m, 1 H), 2.74-3.09 (m, 3 H), 4.46 (dt, J = 47, 5 Hz, 2 | |
| | | H), 4.73 (m, 1 H), 6.49 (d, J = 7 Hz, 1 H), 6.58-6.79 | |
| | | (m, 3 H), 6.80-7.01 (m, 3 H), 7.67 (dd, J = 8, 2 Hz, 1 | |
| | | H), 7.87 (d, J = 2 Hz, 1 H), 11.98-13.51 (m, 1 H) | |
| 19 | A | 1H NMR (400 MHz, DMSO-d6) δ ppm 1.76-2.27 (m, | 504 |
| | | 6 H), 2.32 (ddd, J = 6, 4, 2 Hz, 2 H), 2.89 (br t, J = 7 Hz, | |
| | | 2 H), 3.04-3.46 (m partially hidden, 4 H), 3.52-4.03 | |
| | | (m, 2 H), 4.52 (dt, J = 47, 6 Hz, 2 H), 4.97-5.08 (m, 1 | |
| | | H), 6.64 (t, J = 6 Hz, 1 H), 6.87 (d, J = 8 Hz, 1 H), 6.94 | |
| | | (t, J = 8 Hz, 1 H), 6.98-7.08 (m, 1 H), 7.11-7.28 (m, 5 | |
| | | H), 7.74 (dd, J = 8, 2 Hz, 1 H), 7.91 (d, J = 2 Hz, 1 H), | |
| | | 10.07-11.12 (m, 1 H), 12.89 (br s, 1 H) | |
| 20 | A | 1H NMR (400 MHz, DMSO-d6) δ ppm 1.76-2.31 (m, | 538 |
| | | 8 H), 2.86-3.05 (m, 2 H), 3.07-3.30 (m, 4 H), 3.66 | |
| | | (br s, 2 H), 4.38-4.63 (m, 2 H), 4.91-5.11 (m, 1 H), | |
| | | 6.61-6.78 (m, 1 H), 6.83-6.95 (m, 2 H), 6.96-7.08 | |
| | | (m, 1 H), 7.09-7.28 (m, 3 H), 7.31-7.56 (m, 1 H), | |
| | | 7.76 (dd, J = 8, 2 Hz, 1 H), 7.92 (d, J = 2 Hz, 1 H), 9.92- | |
| | | 11.02 (m, 1 H), 12.91 (br s, 1 H) | |
| 21 | A | 1H NMR (400 MHz, DMSO-d6) δ ppm 1.85-2.35 (m, | 572 |
| | | 8 H), 2.84-3.06 (m, 2 H), 3.06-3.52 (m partially | |
| | | hidden, 4 H), 3.59-4.04 (m, 2 H), 4.36-4.69 (m, 2 | |
| | | H), 5.04 (br s, 1 H), 6.56-6.75 (m, 1 H), 6.89 (br d, | |
| | | J = 8 Hz, 1 H), 6.96 (td, J = 8, 0 Hz, 1 H), 7.00-7.39 (m, | |
| | | 3 H), 7.51-7.69 (m, 1 H), 7.76 (dd, J = 8, 2 Hz, 1 H), | |
| | | 7.93 (d, J = 2 Hz, 1 H), 10.17-11.26 (m, 1 H), 12.93 | |
| | | (br s, 1 H) | |
| 22 | A | 1H NMR (400 MHz, DMSO-d6) δ ppm 1.83 (s, 3 H), | 500 |
| | | 1.89-2.47 (m partially hidden, 8 H), 2.92 ( t, J = 7 Hz, | |
| | | 2 H), 3.01-3.29 (m partially hidden, 4 H), 3.45-3.91 | |
| | | (m, 2 H), 4.44-4.63 (m, 2 H), 4.92 (ddd, J = 5, 3, 1 Hz, | |
| | | 1 H), 6.37-6.55 (m, 1 H), 6.60-6.74 (m, 1 H), 6.81 | |
| | | (d, J = 8 Hz, 1 H), 7.02 (dd, J = 8, 3 Hz, 1 H), 7.08-7.24 | |
| | | (m, 5 H), 7.66-7.79 (m, 1 H), 7.92 (d, J = 1 Hz, 1 H), | |
| | | 9.73-11.06 (m, 1 H), 12.88 (br s, 1 H) | |
| 23 | A | 1H NMR (400 MHz, DMSO-d6) δ ppm 1.47-2.33 (m, | 534 |
| | | 11 H), 2.80-3.30 (m, 6 H), 3.49-4.03 (m, 2 H), 4.43- | |
| | | 4.70 (m, 2 H), 4.78-5.03 (m, 1 H), 6.59 (br dd, J = 7, 2 | |
| | | Hz, 1 H), 6.62-6.77 (m, 1 H), 6.84 (d, J = 8 Hz, 1 H), | |
| | | 6.92-7.04 (m, 1 H), 7.05-7.36 (m, 3 H), 7.46 (br t, | |
| | | J = 9 Hz, 1 H), 7.74 (d, J = 8 Hz, 1 H), 7.94 (d, J = 2 Hz, 1 | |
| | | H), 9.97-11.34 (m, 1 H), 12.91 (br s, 1 H) | |
| 24 | A | 1H NMR (400 MHz, DMSO-d6) δ ppm 1.87-2.40 (m, | 568 |
| | | 11 H), 2.83-3.31 (m, 6 H), 3.49-4.04 (m, 2 H), 4.39- | |
| | | 4.68 (m, 2 H), 4.85-5.07 (m, 1 H), 6.57 (br d, J = 5 Hz, | |
| | | 1 H), 6.65-6.78 (m, 1 H), 6.84 (br d, J = 8 Hz, 1 H), | |
| | | 6.94-7.05 (m, 1 H), 7.06-7.69 (m, 3 H), 7.70-7.78 | |
| | | (m, 1 H), 7.94 (d, J = 2 Hz, 1 H), 9.99-11.13 (m, 1 H), | |
| | | 12.92 (br s, 1 H) | |
| 25 | A | 1H NMR (400 MHz, DMSO-d6) δ ppm 1.92-2.13 (m, | 504 |
| | | 2 H), 2.14-2.24 (m, 2 H), 2.26-2.39 (m, 2 H), 2.51- | |
| | | 2.60 (m hidden, 2 H), 2.89 (t, J = 7 Hz, 2 H), 3.06-3.31 | |
| | | (m partially hidden, 5 H), 3.43-3.86 (m, 1 H), 4.54 | |
| | | (dt, J = 47, 6 Hz, 2 H), 4.85-4.98 (m, 1 H), 6.51-6.63 | |
| | | (m, 1 H), 6.75-6.85 (m, 1 H), 6.92 (d, J = 8 Hz, 1 H), | |
| | | 7.01 (t, J = 9 Hz, 1 H), 7.13-7.27 (m, 5 H), 7.75 (dd, | |
| | | J = 8, 2 Hz, 1 H), 7.91 (d, J = 2 Hz, 1 H), 9.56-10.99 (m, | |
| | | 1 H), 12.89 (s, 1 H) | |
| 26 | A | 1H NMR (400 MHz, DMSO-d6) δ ppm 1.52-2.44 (m, | 538 |
| | | 8 H), 2.86-3.31 (m partially hidden, 6 H), 3.39-4.03 | |
| | | (m, 2 H), 4.55 (br d, J = 47 Hz, 2 H), 4.73-5.01 (m, 1 | |
| | | H), 6.60 (br dd, J = 3, 3 Hz, 1 H), 6.81 (dt, J = 4, 2 Hz, 1 | |
| | | H), 6.91-7.07 (m, 2 H), 7.11-7.34 (m, 3 H), 7.36- | |
| | | 7.59 (m, 1 H), 7.77 (dd, J = 8, 2 Hz, 1 H), 7.93 (d, J = 2 | |
| | | Hz, 1 H), 9.47-11.39 (m, 1 H), 12.92 (br s, 1 H) | |
| 27 | A | 1H NMR (400 MHz, DMSO-d6) δ ppm 1.51-2.34 (m, | 572 |
| | | 8 H), 2.85-3.04 (m, 2 H), 3.06-3.31 (m partially | |
| | | hidden, 4 H), 3.45-3.96 (m, 2 H), 4.54 (dt, J = 47, 5 | |
| | | Hz, 2 H), 4.82-5.11 (m, 1 H), 6.51-6.64 (m, 1 H), | |
| | | 6.84 (dt, J = 5, 2 Hz, 1 H), 6.90-6.97 (m, 1 H), 7.01 (br | |
| | | t, J = 9 Hz, 1 H), 7.11-7.68 (m, 3 H), 7.77 (dd, J = 8, 2 | |
| | | Hz, 1 H), 7.93 (d, J = 2 Hz, 1 H), 9.69-11.36 (m, 1 H), | |
| | | 12.94 (br s, 1 H) | |
| 28 | C | 1H NMR (400 MHz, DMSO-d6) δ ppm 1.57-1.95 (m, | 552 |
| | | 6 H), 2.09-2.31 (m, 5 H), 2.35-2.48 (m, 4 H), 2.62 | |
| | | (m, 1 H), 2.79(m, 1 H), 2.88-3.11 (m, 2 H), 4.33- | |
| | | 4.59 (m, 2 H), 4.74 (br s, 1 H), 6.51-6.64 (m, 1 H), | |
| | | 6.67 (d, J = 8 Hz, 1 H), 6.76 (dd, J = 8, 1 Hz, 1 H), 6.93 | |
| | | (t, J = 8 Hz, 1 H), 7.05-7.41 (m, 2 H), 7.54 (m, 1 H), | |
| | | 7.72 (dd, J = 8, 2 Hz, 1 H), 7.91 (d, J = 2 Hz, 1 H), | |
| | | 11.88-13.97 (m, 1 H) | |
| 29 | A | 1H NMR (400 MHz, DMSO-d6) δ ppm 1.77-1.99 (m, | 506 |
| | | 2 H), 2.06-2.28 (m, 4 H), 2.86-3.03 (m, 2 H), 3.20- | |
| | | 3.30 (m, 2 H), 3.74-4.53 (m, 4 H), 4.52 (dt, J = 47, 6 | |
| | | Hz, 2 H), 4.68-5.01 (m, 1 H), 6.32 (br s, 1 H), 6.54 | |
| | | (d, J = 8 Hz, 1 H), 6.63 (d, J = 7 Hz, 1 H), 6.88 (d, J = 8 | |
| | | Hz, 1 H), 7.10 (t, J = 8 Hz, 1 H), 7.17-7.32 (m, 3 H), | |
| | | 7.45 (br d, J = 8 Hz, 1 H), 7.77 (dd, J = 8, 2 Hz, 1 H), | |
| | | 7.94 (s, 1 H), 10.06-11.34 (m, 1 H), 12.92 (br s, 1 H) | |
| 30 | A | 1H NMR (400 MHz, DMSO-d6) δ ppm 1.75-1.99 (m, | 540 |
| | | 2 H), 2.07-2.27 (m, 4 H), 2.85-3.06 (m, 2 H), 3.21- | |
| | | 3.30 (m, 2 H), 3.83-4.14 (m, 2 H), 4.16-4.50 (m, 2 | |
| | | H), 4.52 (dt, J = 47, 6 Hz, 2 H), 4.74-5.02 (m, 1 H), | |
| | | 6.34 (br s, 1 H), 6.55 (br d, J = 8 Hz, 1 H), 6.66 (d, J = 7 | |
| | | Hz, 1 H), 6.87 (d, J = 8 Hz, 1 H), 7.14 (t, J = 8 Hz, 1 H), | |
| | | 7.21-7.34 (m, 2 H), 7.62 (d, J = 2 Hz, 1 H), 7.76 (dd, | |
| | | J = 8, 2 Hz, 1 H), 7.93 (d, J = 1 Hz, 1 H), 10.00-11.00 | |
| | | (m, 1 H), 12.94 (br s, 1 H) | |
| 31 | A | 1H NMR (400 MHz, DMSO-d6) δ ppm 1.73-1.96 (m, | 538 |
| | | 2 H), 2.07-2.28 (m, 4 H), 2.70-2.88 (m, 3 H), 2.89- | |
| | | 3.05 (m, 2 H), 3.06-3.25 (m, 2 H), 3.47-3.73 (m, 2 | |
| | | H), 3.75-4.00 (m, 2 H), 4.51 (dt, J = 47, 6 Hz, 2 H), | |
| | | 6.70 (s, 1 H), 6.73 (d, J = 8 Hz, 1 H), 6.85 (d, J = 8 Hz, 1 | |
| | | H), 6.98 (d, J = 8 Hz, 1 H), 7.11 (t, J = 7 Hz, 1 H), 7.22 | |
| | | (d, J = 9 Hz, 1 H), 7.28 (dd, J = 8, 2 Hz, 1 H), 7.60 (d, | |
| | | J = 2 Hz, 1 H), 7.76 (dd, J = 8, 2 Hz, 1 H), 7.94 (d, J = 2 | |
| | | Hz, 1 H), 10.18 (br s, 1 H), 12.94 (br s, 1 H) | |
| 32 | D | 1H NMR (400 MHz, DMSO-d6) δ ppm 1.74-2.01 (m, | 539 |
| | | 2 H), 2.07-2.25 (m, 4 H), 2.93 (br d, J = 3 Hz, 2 H), | |
| | | 3.19-3.31 (m, 2 H), 3.55-4.42 (m, 5 H), 4.52 (dt, | |
| | | J = 47, 6 Hz, 2 H), 6.03 (br d, J = 1 Hz, 1 H), 6.16-6.24 | |
| | | (m, 2 H), 6.31 (br d, J = 7 Hz, 1 H), 6.85-6.97 (m, 2 | |
| | | H), 7.20 (d, J = 9 Hz, 1 H), 7.28 (dd, J = 8, 3 Hz, 1 H), | |
| | | 7.62 (d, J = 2 Hz, 1 H), 7.76 (dd, J = 8, 2 Hz, 1 H), 7.92 | |
| | | (d, J = 2 Hz, 1 H), 10.16 (br s, 1 H), 12.91 (br s, 1 H) | |
| 33 | D | 1H NMR (400 MHz, DMSO-d6) δ ppm 1.81 (m, 1 H), | 553 |
| | | 1.93-2.25 (m, 7 H), 2.43-2.46 (m, 2 H), 2.85-2.98 | |
| | | (m, 2 H), 3.02-3.53 (m, 4 H), 3.80-4.01 (m, 1 H), | |
| | | 4.53 (dt, J = 47, 5 Hz, 2 H), 5.73-5.94 (m, 1 H), 6.10 | |
| | | (br s, 1 H), 6.17 (m, 1 H), 6.37 (d, J = 8 Hz, 1 H), 6.84- | |
| | | 6.99 (m, 2 H), 7.15-7.34 (m, 2 H), 7.61 (s, 1 H), 7.76 | |
| | | (br d, J = 8 Hz, 1 H), 7.91 (s, 1 H), 10.20 (br s, 1 H), | |
| | | 12.69-13.02 (m, 1 H) | |
| 34 | F | 1H NMR (400 MHz, DMSO-d6) δ ppm 1.39-1.68 (m, | 526 |
| | | 1 H), 1.71-1.89 (m, 2 H), 1.95-2.25 (m, 5 H), 2.30- | |
| | | 2.88 (m, 9 H), 4.47 (dt, J = 47, 6 Hz, 1 H), 4.58 (m, 1 | |
| | | H), 6.33 (d, J = 2 Hz, 1 H), 6.40(m, 1 H), 6.57 (d, J = 1 | |
| | | Hz, 2 H), 6.60 (ddd, J = 8, 3, 1 Hz, 1 H), 6.72 (t, J = 1 | |
| | | Hz, 1 H), 7.02 (t, J = 8 Hz, 1 H), 7.14 (m, 1 H), 7.25 (m, | |
| | | 1 H), 7.56 (d, J = 2 Hz, 1 H), 9.44 (s, 1 H) | |
| 35 | E | 1H NMR (400 MHz, DMSO-d6) δ ppm 1.68-2.19 (m, | 556 |
| | | 3 H), 2.50 (m partially hidden, 6 H), 3.01-3.27 (m | |
| | | hidden, 4 H), 3.64 (m, 2 H), 4.34-4.72 (m, 3 H), | |
| | | 4.88-5.10 (m, 1 H), 6.48 (br s, 1 H), 6.57 (br d, J = 8 | |
| | | Hz, 1 H), 6.78 (m, 1 H), 6.86 (d, J = 8 Hz, 1 H), 7.07- | |
| | | 7.22 (m, 2 H), 7.28 (dd, J = 8, 2 Hz, 1 H), 7.63 (s, 2 H), | |
| | | 10.04-10.73 (m, 1 H) | |
| 36 | G | 1H NMR (400 MHz, DMSO-d6) δ ppm 0.74 (d, J = 7 | 534 |
| | | Hz, 3 H), 1.52-1.67 (m, 1 H), 1.69-2.04 (m, 3 H), | |
| | | 2.08-2.29 (m, 2 H), 2.50 (m partially hidden, 7 H), | |
| | | 2.80-2.87 (m, 1 H), 2.99-3.11 (m, 1 H), 4.48 (dt, | |
| | | J = 48, 6 Hz, 2 H), 4.65-4.76 (m, 1 H), 6.45-6.54 (m, | |
| | | 2 H), 6.59 (d, J = 8 Hz, 1 H), 6.95 (d, J = 8 Hz, 1 H), 7.02 | |
| | | (t, J = 8 Hz, 1 H), 7.12 (d, J = 7 Hz, 1 H), 7.16-7.28 (m, | |
| | | 3 H), 7.75 (dd, J = 8, 2 Hz, 1 H), 7.90 (s, 1 H), 12.01- | |
| | | 13.35 (m, 1 H) | |
| 37 | G | 1H NMR (400 MHz, DMSO-d6) δ ppm 0.74 (d, J = 7 | 534 |
| | | Hz, 3 H), 1.66-1.71 (m, 1 H), 1.71-1.88 (m, 2 H), | |
| | | 1.92-2.04 (m, 1 H), 2.13-2.29 (m, 2 H), 2.38-2.47 | |
| | | (m, 2 H), 2.58 (m partially hidden, 1 H), 2.69-2.91 | |
| | | (m, 4 H), 2.96-3.15 (m, 2 H), 4.48 (dt, J = 47, 6 Hz, 2 | |
| | | H), 4.69 (m, 1 H), 6.45-6.52 (m, 2 H), 6.59 (m, 1 H), | |
| | | 6.95 (d, J = 8 Hz, 1 H), 7.02 (t, J = 6 Hz, 1 H), 7.11-7.31 | |
| | | (m, 4 H), 7.75 (dd, J = 8, 2 Hz, 1 H), 7.90 (d, J = 1 Hz, 1 | |
| | | H), 11.60-14.16 (m, 1 H) | |
| 38 | A | 1H NMR (400 MHz, DMSO-d6) δ ppm 1.62 (m, 1 H), | 510 |
| | | 1.71-1.88 (m, 2 H), 1.98-2.24 (m, 5 H), 2.36-2.44 | |
| | | (m, 2 H), 2.45-2.48 (m hidden, 2 H), 2.56-2.75 (m, 3 | |
| | | H), 2.84-2.92 (m, 2 H), 4.46 (dt, J = 47, 6 Hz, 2 H), | |
| | | 4.56 (m, 1 H), 6.38 (d, J = 3 Hz, 1 H), 6.43 (m, 1 H), | |
| | | 6.62 (ddd, J = 8, 3, 1 Hz, 1 H), 6.80 (dd, J = 8, 2 Hz, 1 | |
| | | H), 7.02 (t, J = 8 Hz, 1 H), 7.10-7.27 (m, 3 H), 7.31 | |
| | | (dd, J = 7, 1 Hz, 1 H), 7.50 (d, J = 2 Hz, 1 H) | |
| 39 | A | 1H NMR (400 MHz, DMSO-d6) δ ppm 1.42-1.68 (m, | 476 |
| | | 1 H), 1.70-1.91 (m, 2 H), 1.95-2.25 (m, 5 H), 2.28- | |
| | | 2.69 (m partially hidden, 6 H), 2.78-3.00 (m, 2 H), | |
| | | 4.47 (dt, J = 46, 6 Hz, 2 H), 4.47-4.50 (m, 1 H), 6.36 | |
| | | (s, 1 H), 6.40 (d, J = 8 Hz, 1 H), 6.59 (dd, J = 8, 2 Hz, 1 | |
| | | H), 6.79 (dd, J = 8, 1 Hz, 1 H), 7.01 (t, J = 8 Hz, 1 H), | |
| | | 7.12-7.27 (m, 5 H), 7.33 (d, J = 6 Hz, 1 H), 7.42 (br d, | |
| | | J = 7 Hz, 1 H) | |
| 40 | E | 1H NMR (400 MHz, DMSO-d6) δ ppm 1.87-2.19 (m, | 540 |
| | | 3 H), 2.51-2.76 (m partially hidden, 6 H), 3.03-3.09 | |
| | | (m, 2 H), 3.11-3.28 (m partially hidden, 3 H), 4.54 | |
| | | (dt, J = 47, 6 Hz, 2 H), 4.97-5.14 (m, 1 H), 6.61-6.88 | |
| | | (m, 4 H), 7.16-7.30 (m, 3 H), 7.55 (d, J = 2 Hz, 1 H), | |
| | | 7.70 (dd, J = 8, 2 Hz, 1 H), 7.85 (d, J = 2 Hz, 1 H), | |
| | | 10.01-10.64 (m, 1 H), 12.85 (br s, 1 H) | |
| 41 | F | 1H NMR (400 MHz, DMSO-d6) δ ppm 1.40-1.90 (m, | 544 |
| | | 3 H), 1.97-2.21 (m, 1 H), 2.27-2.87 (m partially | |
| | | hidden, 8 H), 3.12 (m, 1 H), 3.43 (m, 1 H), 4.47 (dt, | |
| | | J = 48, 6 Hz, 2 H), 4.58 (m, 1 H), 6.29-6.42 (m, 3 H), | |
| | | 6.64 (br d, J = 8 Hz, 1 H), 6.70 (br d, J = 8 Hz, 1 H), | |
| | | 7.03-7.14 (m, 2 H), 7.27 (br d, J = 8 Hz, 1 H), 7.44 | |
| | | (br d, J = 8 Hz, 1 H), 7.66 (br s, 1 H), 9.61 (s, 1 H) | |

The examples which follow describe the preparation of some compounds of formula (I) described herein. The numbers of the compounds exemplified below match those given in the Tables 1a and 1b above. All reactions are performed under inert atmosphere, unless otherwise stated.

In the following examples, when the source of the starting products is not specified, it should be understood that said products are known compounds.

### INTERMEDIATES

### Intermediate 1: (R)-1-(3-Fluoropropyl)-3-[3-(4,4,5,5-tetramethyl-1,3,2-dioxaborolan-2-yl)phenoxy]pyrrolidine

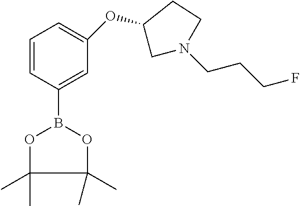 [SMILES: CC1(C)OB(c2cccc(O[C@@H]3CCN(CCCF)C3)c2)OC1(C)C]

### Step 1: Tert-butyl (R)-3-(3-(4,4,5,5-tetramethyl-1,3,2-dioxaborolan-2-yl)phenoxy)pyrrolidine-1-carboxylate

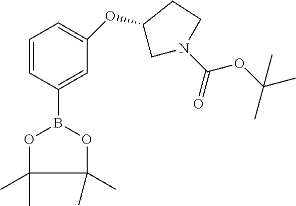 [SMILES: CC(C)(C)OC(=O)N1CC[C@@H](Oc2cccc(B3OC(C)(C)C(C)(C)O3)c2)C1]

To a mixture of 3-(4,4,5,5-tetramethyl-1,3,2-dioxaborolan-2-yl)phenol (1.01 g, 4.50 mmol), (S)-1-N-boc-3-hydroxypyrrolidine (0.97 g, 5.08 mmol), and N,N,N′,N′-tetramethylazodicarboxamide (1.25 g, 7.11 mmol) in THF (20 ml) was added PPh₃ (1.84 g, 6.87 mmol). The reaction mixture was stirred at RT for 18 hours. Diisopropyl ether (50 ml) and water (30 ml) were added. The organic phase was washed with water (50 ml), dried over Na₂SO₄ filtered and concentrated under reduced pressure and the residue obtained by flash chromatography, eluting with heptane/EtOAc: 80/20 to give 1.36 g (78%) of tert-butyl (R)-3-(3-(4,4,5,5-tetramethyl-1,3,2-dioxaborolan-2-yl)phenoxy)pyrrolidine-1-carboxylate.

LC/MS (m/z, MH+): 390

### Step 2: (R)-3-(3-(4,4,5,5-Tetramethyl-1,3,2-dioxaborolan-2-yl)phenoxy)pyrrolidine, hydrochloride

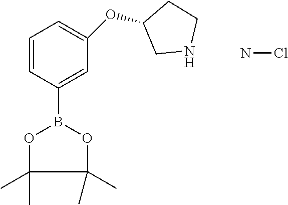

To a solution of tert-butyl (R)-3-(3-(4,4,5,5-tetramethyl-1,3,2-dioxaborolan-2-yl)phenoxy)pyrrolidine-1-carboxylate (1.03, 2.65 mmol) in MeOH (10 ml) cooled at 5° C., was added HCl 4M in dioxane (5.2 ml, 20.80 mmol). The reaction mixture was stirred at RT for 2 hours. The reaction mixture was concentrated under reduced pressure and the residue obtained was triturated with diisopropyl ether. The solid was filtered and dried to give 0.76 g (99%) of (R)-3-(3-(4,4,5,5-tetramethyl-1,3,2-dioxaborolan-2-yl)phenoxy)pyrrolidine, hydrochloride.

LC/MS (m/z, MH+): 290

### Step 3: (R)-1-(3-fluoropropyl)-3-(3-(4,4,5,5-tetramethyl-1,3,2-dioxaborolan-2-yl)phenoxy)pyrrolidine

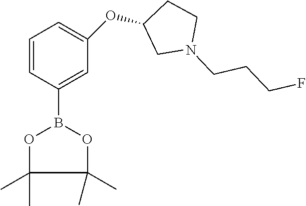 [SMILES: CC1(C)OB(c2cccc(O[C@@H]3CCN(CCCF)C3)c2)OC1(C)C]

A mixture of (R)-3-(3-(4,4,5,5-tetramethyl-1,3,2-dioxaborolan-2-yl)phenoxy)pyrrolidine, hydrochloride (0.75 g, 2.62 mmol), K₂CO₃ (0.73 g, 5.24 mmol), 1-fluoro-3-iodo-propane (0.475 g, 2.48 mmol) in MeCN (7 ml) was heated at 70° C. for 2 hours. After cooling to RT, DCM (20 ml) and water (10 ml) were added. The organic phase was washed over Na₂SO₄ filtered and concentrated under reduced pressure and the residue obtained was purified by flash chromatography, eluting with DCM/MeOH: 95/05 to give 0.4 g (44%) of (R)-1-(3-fluoropropyl)-3-(3-(4,4,5,5-tetramethyl-1,3,2-dioxaborolan-2-yl)phenoxy)pyrrolidine.

LC/MS (m/z, MH+): 350

### Intermediate 2: Methyl (R)-8-bromo-9-(3-((1-(3-fluoropropyl)pyrrolidin-3-yl)oxy)phenyl)-6,7-dihydro-5H-benzo[7]annulene-3-carboxylate

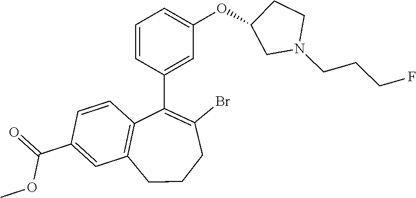 [SMILES: COC(=O)c1ccc2c(c1)CCCC(Br)=C2c1cccc(O[C@@H]2CCN(CCCF)C2)c1]

### Step 1: Methyl 9-(3-hydroxyphenyl)-6,7-dihydro-5H-benzo[7]annulene-3-carboxylate

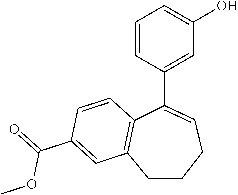 [SMILES: COC(=O)c1ccc2c(c1)CCCC=C2c1cccc(O)c1]

A mixture of methyl 9-(((trifluoromethyl)sulfonyl)oxy)-6,7-dihydro-5H-benzo[7]annulene-3-carboxylate (25 g, 71.37 mol) (prepared according to WO2017140669), 3-(4,4,5,5-tetramethyl-1,3,2-dioxaborolan-2-yl)phenol (17.28 g, 78.50 mol), Cs₂CO₃ (46.55 g, 142.73 mmol), Pd(dppf)Cl₂ complex with DCM (4.70 g, 6.42 mmol), in dioxane (204 ml) and water (72 ml) was stirred at RT for 30 minutes. The reaction mixture was quenched by addition of water (500 ml) and extracted with EtOAc (2×500 ml). The organic phase was dried over Na₂SO₄ filtered and concentrated under reduced pressure and the residue obtained by flash chromatography, eluting with heptane/EtOAc: 80/20 to give 20.5 g (98%) of methyl 9-(3-hydroxyphenyl)-6,7-dihydro-5H-benzo[7]annulene-3-carboxylate.

LC/MS (m/z, MH+): 295

### Step 2: Methyl (R)-9-(3-((1-(3-fluoropropyl)pyrrolidin-3-yl)oxy)phenyl)-6,7-dihydro-5H-benzo[7]annulene-3-carboxylate

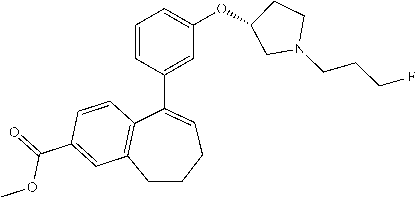 [SMILES: COC(=O)c1ccc2c(c1)CCCC=C2c1cccc(O[C@@H]2CCN(CCCF)C2)c1]

### Method 1

To a mixture of methyl 9-(3-hydroxyphenyl)-6,7-dihydro-5H-benzo[7]annulene-3-carboxylate (0.5 g, 1.70 mol), (S)-1-(3-fluoropropyl)pyrrolidin-3-ol (0.276 g, 1.88 mol) (prepared according WO2018091153), and N,N,N′,N′-tetramethylazodicarboxamide (0.39 g, 2.23 mmol) in THF (5 ml) was added PPh₃ (0.59 g, 2.21 mmol). The reaction mixture was stirred at RT for 18 hours. Addition of diisopropyl ether (20 ml) and water (5 ml). The organic phase was washed with water (50 ml), dried over Na₂SO₄ filtered and concentrated under reduced pressure to give 0.86 g (crude) of methyl (R)-9-(3-((1-(3-fluoropropyl)pyrrolidin-3-yl)oxy)phenyl)-6,7-dihydro-5H-benzo[7]annulene-3-carboxylate which was used as such in the next step.

LC/MS (m/z, MH+): 424

### Method 2

A mixture of methyl 9-(((trifluoromethyl)sulfonyl)oxy)-6,7-dihydro-5H-benzo[7]annulene-3-carboxylate (0.72 g, 2.06 mmol) (prepared according to WO2017140669), (R)-1-(3-fluoropropyl)-3-(3-(4,4,5,5-tetramethyl-1,3,2-dioxaborolan-2-yl)phenoxy)pyrrolidine (Intermediate 1) (0.63 g, 1.79 mmol), Cs₂CO₃ (1.55 g, 4.66 mmol), Pd(dppf)Cl₂ complex with DCM (91 mg, 0.1 mmol), in dioxane (8 ml) and water (2 ml) was stirred at RT for 20 minutes. The reaction mixture was quenched by addition of water (5 ml) and DCM (10 ml) and extracted with hydrophobic column. The organic phase was dried over Na₂SO₄ filtered and concentrated under reduced pressure and the residue obtained by flash chromatography, eluting with DCM/MeOH: 95/05 to give 0.75 g (99%) of methyl (R)-9-(3-((1-(3-fluoropropyl)pyrrolidin-3-yl)oxy)phenyl)-6,7-dihydro-5H-benzo[7]annulene-3-carboxylate.

### Step 3: Methyl (R)-8-bromo-9-(3-((1-(3-fluoropropyl)pyrrolidin-3-yl)oxy)phenyl)-6,7-dihydro-5H-benzo[7]annulene-3-carboxylate

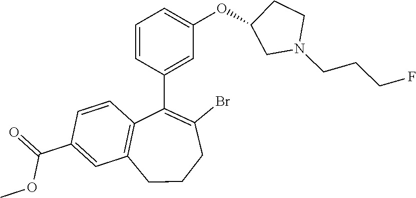 [SMILES: COC(=O)c1ccc2c(c1)CCCC(Br)=C2c1cccc(O[C@@H]2CCN(CCCF)C2)c1]

To a mixture of methyl (R)-9-(3-((1-(3-fluoropropyl)pyrrolidin-3-yl)oxy)phenyl)-6,7-dihydro-5H-benzo[7]annulene-3-carboxylate (0.75 g, 1.77 mmol) in DCM (8 ml) was added pyridinium tribromide (0.69 g, 1.95 mmol). The reaction mixture was stirred for 1.5 hours at room temperature. The reaction was quenched with concentrated solution of NaHCO₃ and extracted with hydrophobic column. The organic phase was dried over Na₂SO₄, filtered, concentrated under reduced pressure and the residue obtained was purified by flash chromatography eluting with a gradient of DCM/MeOH: from 100/00 to 90/10 to give 0.48 g (54%) of methyl (R)-8-bromo-9-(3-((1-(3-fluoropropyl)pyrrolidin-3-yl)oxy)phenyl)-6,7-dihydro-5H-benzo[7]annulene-3-carboxylate.

LC/MS (m/z, MH+): 502

### Intermediate 3: Methyl (R)-8-bromo-9-(2-fluoro-5-((1-(3-fluoropropyl)pyrrolidin-3-yl)oxy)phenyl)-6,7-dihydro-5H-benzo[7]annulene-3-carboxylate

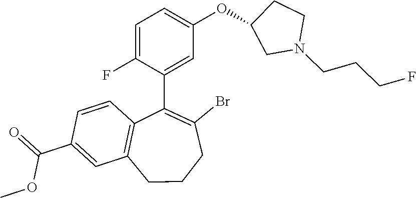 [SMILES: COC(=O)c1ccc2c(c1)CCCC(Br)=C2c1cc(O[C@@H]2CCN(CCCF)C2)ccc1F]

### Step 1: Methyl 9-(2-fluoro-5-hydroxyphenyl)-6,7-dihydro-5H-benzo[7]annulene-3-carboxylate

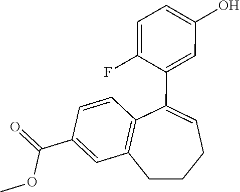 [SMILES: COC(=O)c1ccc2c(c1)CCCC=C2c1cc(O)ccc1F]

Step 1 of Intermediate 3 was prepared following a similar procedure to that of Step 1 Intermediate 2 from methyl 9-(((trifluoromethyl)sulfonyl)oxy)-6,7-dihydro-5H-benzo[7]annulene-3-carboxylate (prepared according to WO2017140669) and 2-fluoro-5-hydroxyphenylboronic acid to give 1.52 g (85%) of methyl 9-(2-fluoro-5-hydroxyphenyl)-6,7-dihydro-5H-benzo[7]annulene-3-carboxylate.

LC/MS (m/z, MH+): 313

### Step 2: Methyl (R)-9-(2-fluoro-5-((1-(3-fluoropropyl)pyrrolidin-3-yl)oxy)phenyl)-6,7-dihydro-5H-benzo[7]annulene-3-carboxylate

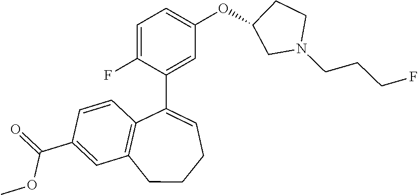 [SMILES: COC(=O)c1ccc2c(c1)CCCC=C2c1cc(O[C@@H]2CCN(CCCF)C2)ccc1F]

Step 2 of Intermediate 3 was prepared following a similar procedure to that of Step 2 Method 1 of Intermediate 2 from methyl 9-(2-fluoro-5-hydroxyphenyl)-6,7-dihydro-5H-benzo[7]annulene-3-carboxylate and (S)-1-(3-fluoropropyl)pyrrolidin-3-ol (prepared according WO2018091153), to give 0.7 g (99%) of methyl (R)-9-(2-fluoro-5-((1-(3-fluoropropyl)pyrrolidin-3-yl)oxy)phenyl)-6,7-dihydro-5H-benzo[7]annulene-3-carboxylate.

LC/MS (m/z, MH+): 442

### Step 3: Methyl (R)-8-bromo-9-(2-fluoro-5-((1-(3-fluoropropyl)pyrrolidin-3-yl)oxy)phenyl)-6,7-dihydro-5H-benzo[7]annulene-3-carboxylate

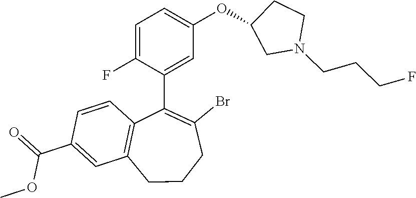 [SMILES: COC(=O)c1ccc2c(c1)CCCC(Br)=C2c1cc(O[C@@H]2CCN(CCCF)C2)ccc1F]

Step 3 of Intermediate 3 was prepared following a similar procedure to that of Step 3 of Intermediate 2 from methyl (R)-9-(2-fluoro-5-((1-(3-fluoropropyl)pyrrolidin-3-yl)oxy)phenyl)-6,7-dihydro-5H-benzo[7]annulene-3-carboxylate to give 0.36 g (42%) of methyl (R)-8-bromo-9-(2-fluoro-5-((1-(3-fluoropropyl)pyrrolidin-3-yl)oxy)phenyl)-6,7-dihydro-5H-benzo[7]annulene-3-carboxylate.

LC/MS (m/z, MH+): 520

### Intermediate 4: Methyl (R)-8-bromo-9-(5-((1-(3-fluoropropyl)pyrrolidin-3-yl)oxy)-2-methylphenyl)-6,7-dihydro-5H-benzo[7]annulene-3-carboxylate

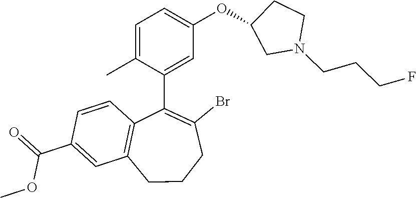 [SMILES: COC(=O)c1ccc2c(c1)CCCC(Br)=C2c1cc(O[C@@H]2CCN(CCCF)C2)ccc1C]

### Step 1: Methyl 9-(5-hydroxy-2-methylphenyl)-6,7-dihydro-5H-benzo[7]annulene-3-carboxylate

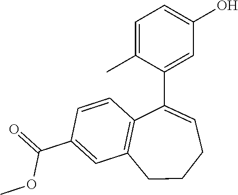 [SMILES: COC(=O)c1ccc2c(c1)CCCC=C2c1cc(O)ccc1C]

Step 1 of Intermediate 4 was prepared following a similar procedure to that of Step 1 Intermediate 2 from methyl 9-(((trifluoromethyl)sulfonyl)oxy)-6,7-dihydro-5H-benzo[7]annulene-3-carboxylate (prepared according to WO2017140669) and 4-methyl-3-(4,4,5,5-tetramethyl-1,3,2-dioxaborolan-2-yl)phenol to give 1.43 g (81%) of methyl 9-(5-hydroxy-2-methylphenyl)-6,7-dihydro-5H-benzo[7]annulene-3-carboxylate.

LC/MS (m/z, MH+): 309

### Step 2: Methyl (R)-9-(5-((1-(3-fluoropropyl)pyrrolidin-3-yl)oxy)-2-methylphenyl)-6,7-dihydro-5H-benzo[7]annulene-3-carboxylate

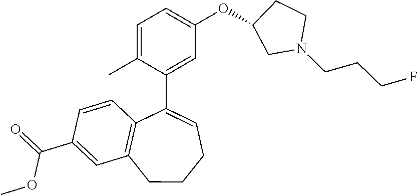 [SMILES: COC(=O)c1ccc2c(c1)CCCC=C2c1cc(O[C@@H]2CCN(CCCF)C2)ccc1C]

Step 2 of Intermediate 4 was prepared following a similar procedure to that of Step 2 Method 1 of Intermediate 2 from methyl 9-(5-hydroxy-2-methylphenyl)-6,7-dihydro-5H-benzo[7]annulene-3-carboxylate and (S)-1-(3-fluoropropyl)pyrrolidin-3-ol (prepared according WO2018091153), to give 0.7 g (99%) of methyl (R)-9-(5-((1-(3-fluoropropyl)pyrrolidin-3-yl)oxy)-2-methylphenyl)-6,7-dihydro-5H-benzo[7]annulene-3-carboxylate.

LC/MS (m/z, MH+): 438

### Step 3: Methyl (R)-8-bromo-9-(5-((1-(3-fluoropropyl)pyrrolidin-3-yl)oxy)-2-methylphenyl)-6,7-dihydro-5H-benzo[7]annulene-3-carboxylate

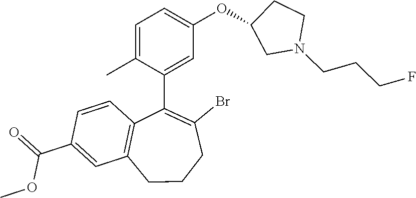 [SMILES: COC(=O)c1ccc2c(c1)CCCC(Br)=C2c1cc(O[C@@H]2CCN(CCCF)C2)ccc1C]

Step 3 of Intermediate 4 was prepared following a similar procedure to that of Step 3 of Intermediate 2 from methyl (R)-9-(5-((1-(3-fluoropropyl)pyrrolidin-3-yl)oxy)-2-methylphenyl)-6,7-dihydro-5H-benzo[7]annulene-3-carboxylate to give 0.34 g (41%) of methyl (R)-8-bromo-9-(5-((1-(3-fluoropropyl)pyrrolidin-3-yl)oxy)-2-methylphenyl)-6,7-dihydro-5H-benzo[7]annulene-3-carboxylate.

LC/MS (m/z, MH+): 516

### Intermediate 5: Methyl (R)-8-bromo-9-(2-fluoro-3-((1-(3-fluoropropyl)pyrrolidin-3-yl)oxy)phenyl)-6,7-dihydro-5H-benzo[7]annulene-3-carboxylate

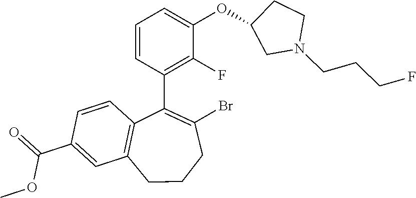 [SMILES: COC(=O)c1ccc2c(c1)CCCC(Br)=C2c1cccc(O[C@@H]2CCN(CCCF)C2)c1F]

### Step 1: Methyl 9-(2-fluoro-3-hydroxyphenyl)-6,7-dihydro-5H-benzo[7]annulene-3-carboxylate

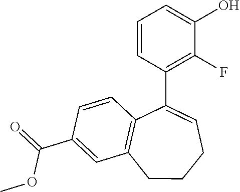 [SMILES: COC(=O)c1ccc2c(c1)CCCC=C2c1cccc(O)c1F]

Step 1 of Intermediate 5 was prepared following a similar procedure to that of Step 1 Intermediate 2 from methyl 9-(((trifluoromethyl)sulfonyl)oxy)-6,7-dihydro-5H-benzo[7]annulene-3-carboxylate (prepared according to WO2017140669) and 2-fluoro-3-hydroxyphenylboronic acid to give 1.15 g (86%) of methyl 9-(2-fluoro-3-hydroxyphenyl)-6,7-dihydro-5H-benzo[7]annulene-3-carboxylate.

LC/MS (m/z, MH+): 313

### Step 2: Methyl (R)-9-(2-fluoro-3-((1-(3-fluoropropyl)pyrrolidin-3-yl)oxy)phenyl)-6,7-dihydro-5H-benzo[7]annulene-3-carboxylate

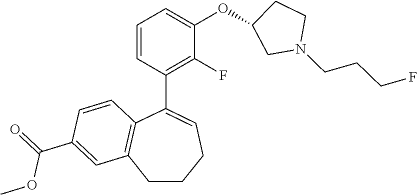 [SMILES: COC(=O)c1ccc2c(c1)CCCC=C2c1cccc(O[C@@H]2CCN(CCCF)C2)c1F]

Step 2 of Intermediate 5 was prepared following a similar procedure to that of Step 2 Method 1 of Intermediate 2 from methyl 9-(2-fluoro-3-hydroxyphenyl)-6,7-dihydro-5H-benzo[7]annulene-3-carboxylate and (S)-1-(3-fluoropropyl)pyrrolidin-3-ol (prepared according WO2018091153), to give 1.5 g (92%) of methyl (R)-9-(2-fluoro-3-((1-(3-fluoropropyl)pyrrolidin-3-yl)oxy)phenyl)-6,7-dihydro-5H-benzo[7]annulene-3-carboxylate.

LC/MS (m/z, MH+): 442

### Step 3: Methyl (R)-8-bromo-9-(2-fluoro-3-((1-(3-fluoropropyl)pyrrolidin-3-yl)oxy)phenyl)-6,7-dihydro-5H-benzo[7]annulene-3-carboxylate

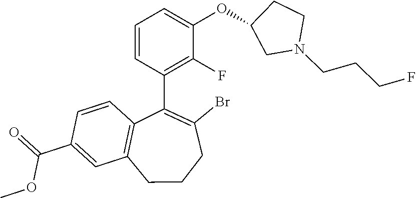 [SMILES: COC(=O)c1ccc2c(c1)CCCC(Br)=C2c1cccc(O[C@@H]2CCN(CCCF)C2)c1F]

Step 3 of Intermediate 5 was prepared following a similar procedure to that of Step 3 of Intermediate 2 from methyl (R)-9-(2-fluoro-5-((1-(3-fluoropropyl)pyrrolidin-3-yl)oxy)phenyl)-6,7-dihydro-5H-benzo[7]annulene-3-carboxylate to give 0.4 g (23%) of methyl (R)-8-bromo-9-(2-fluoro-3-((1-(3-fluoropropyl)pyrrolidin-3-yl)oxy)phenyl)-6,7-dihydro-5H-benzo[7]annulene-3-carboxylate.

LC/MS (m/z, MH+): 520

### Intermediate 6: Methyl (R)-8-bromo-9-(3-((1-(3-fluoropropyl)pyrrolidin-3-yl)oxy)-2-methylphenyl)-6,7-dihydro-5H-benzo[7]annulene-3-carboxylate

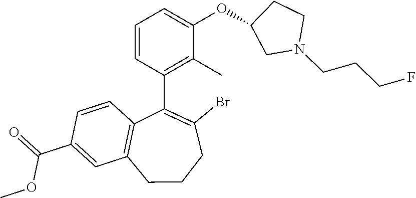 [SMILES: COC(=O)c1ccc2c(c1)CCCC(Br)=C2c1cccc(O[C@@H]2CCN(CCCF)C2)c1C]

### Step 1: Methyl 9-(3-hydroxy-2-methylphenyl)-6,7-dihydro-5H-benzo[7]annulene-3-carboxylate

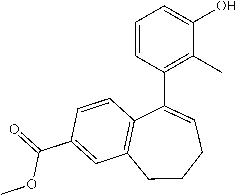

Step 1 of Intermediate 6 was prepared following a similar procedure to that of Step 1 Intermediate 2 from methyl 9-(((trifluoromethyl)sulfonyl)oxy)-6,7-dihydro-5H-benzo[7]annulene-3-carboxylate (prepared according to WO2017140669) and 2-methyl-3-(4,4,5,5-tetramethyl-1,3,2-dioxaborolan-2-yl)phenol to give 1.15 g (87%) of methyl 9-(3-hydroxy-2-methylphenyl)-6,7-dihydro-5H-benzo[7]annulene-3-carboxylate.

LC/MS (m/z, MH+): 309

### Step 2: Methyl (R)-9-(3-((1-(3-fluoropropyl)pyrrolidin-3-yl)oxy)-2-methylphenyl)-6,7-dihydro-5H-benzo[7]annulene-3-carboxylate

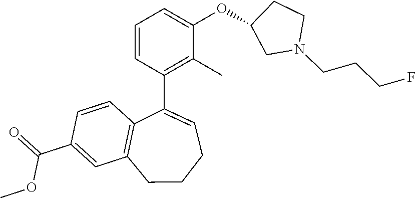 [SMILES: COC(=O)c1ccc2c(c1)CCCC=C2c1cccc(O[C@@H]2CCN(CCCF)C2)c1C]

Step 2 of Intermediate 6 was prepared following a similar procedure to that of Step 2 Method 1 of Intermediate 2 from methyl 9-(5-hydroxy-2-methylphenyl)-6,7-dihydro-5H-benzo[7]annulene-3-carboxylate and (S)-1-(3-fluoropropyl)pyrrolidin-3-ol (prepared according WO2018091153), to give 1.5 g (92%) of methyl (R)-9-(3-((1-(3-fluoropropyl)pyrrolidin-3-yl)oxy)-2-methylphenyl)-6,7-dihydro-5H-benzo[7]annulene-3-carboxylate.

LC/MS (m/z, MH+): 438

### Step 3: Methyl (R)-8-bromo-9-(3-((1-(3-fluoropropyl)pyrrolidin-3-yl)oxy)-2-methylphenyl)-6,7-dihydro-5H-benzo[7]annulene-3-carboxylate

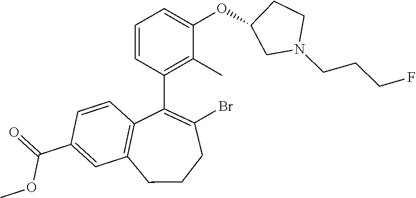 [SMILES: COC(=O)c1ccc2c(c1)CCCC(Br)=C2c1cccc(O[C@@H]2CCN(CCCF)C2)c1C]

Step 3 of Intermediate 6 was prepared following a similar procedure to that of Step 3 of Intermediate 2 from methyl (R)-9-(3-((1-(3-fluoropropyl)pyrrolidin-3-yl)oxy)-2-methylphenyl)-6,7-dihydro-5H-benzo[7]annulene-3-carboxylate to give 0.58 g (38%) of methyl (R)-8-bromo-9-(3-((1-(3-fluoropropyl)pyrrolidin-3-yl)oxy)-2-methylphenyl)-6,7-dihydro-5H-benzo[7]annulene-3-carboxylate.

LC/MS (m/z, MH+): 516

### Intermediate 7: Methyl 8-bromo-9-(3-((1-(3-fluoropropyl)azetidin-3-yl)oxy)phenyl)-6,7-dihydro-5H-benzo[7]annulene-3-carboxylate

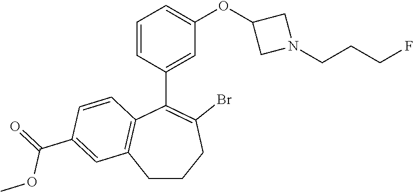 [SMILES: COC(=O)c1ccc2c(c1)CCCC(Br)=C2c1cccc(OC2CN(CCCF)C2)c1]

### Step 1: Tert-butyl 3-(3-(4,4,5,5-tetramethyl-1,3,2-dioxaborolan-2-yl)phenoxy)azetidine-1-carboxylate

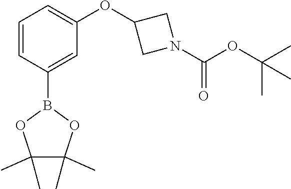 [SMILES: CC(C)(C)OC(=O)N1CC(Oc2cccc(B3OC(C)(C)C(C)(C)O3)c2)C1]

To a mixture of 3-(4,4,5,5-tetramethyl-1,3,2-dioxaborolan-2-yl)phenol (13 g, 59.07 mmol), 1-boc-3-iodoazetidine (25.86 g, 88.61 mmol) DMF (150 ml) was added Cs₂CO₃ (38.53 g, 118.14 mmol). The reaction mixture was stirred at 65° C. for 20 hours. After cooling to RT, the reaction mixture was poured into water (1500 ml) and EtOAc (500 ml). The organic phase was dried over Na₂SO₄ filtered and concentrated under reduced pressure and the residue obtained was triturated with isopropyl ether. The solid was filtered and dried to give 10.6 g (48%) of tert-butyl 3-(3-(4,4,5,5-tetramethyl-1,3,2-dioxaborolan-2-yl)phenoxy)azetidine-1-carboxylate.

LC/MS (m/z, MH+): 376

### Step 2: (R)-3-(3-(4,4,5,5-Tetramethyl-1,3,2-dioxaborolan-2-yl)phenoxy)pyrrolidine, hydrochloride

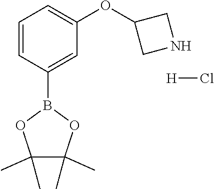

Step 2 of Intermediate 7 was prepared following a similar procedure to that of Step 2 of Intermediate 1 from tert-butyl 3-(3-(4,4,5,5-tetramethyl-1,3,2-dioxaborolan-2-yl)phenoxy)azetidine-1-carboxylate to give 9.1 g (crude) of tert-butyl 3-(3-(4,4,5,5-tetramethyl-1,3,2-dioxaborolan-2-yl)phenoxy)azetidine-1-carboxylate hydrochloride.

LC/MS (m/z, MH+): 276

### Step 3: 1-(3-Fluoropropyl)-3-(3-(4,4,5,5-tetramethyl-1,3,2-dioxaborolan-2-yl)phenoxy)azetidine

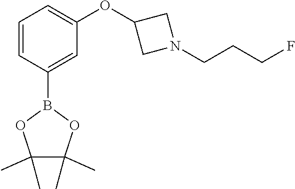 [SMILES: CC1(C)OB(c2cccc(OC3CN(CCCF)C3)c2)OC1(C)C]

Step 3 of Intermediate 7 was prepared following a similar procedure to that of Step 3 of Intermediate 1 from tert-butyl 3-(3-(4,4,5,5-tetramethyl-1,3,2-dioxaborolan-2-yl)phenoxy)azetidine-1-carboxylate hydrochloride to give 1.53 g (16%) of 1-(3-fluoropropyl)-3-(3-(4,4,5,5-tetramethyl-1,3,2-dioxaborolan-2-yl)phenoxy)azetidine.

LC/MS (m/z, MH+): 336

### Step 4: Methyl 9-(3-((1-(3-fluoropropyl)azetidin-3-yl)oxy)phenyl)-6,7-dihydro-5H-benzo[7]annulene-3-carboxylate

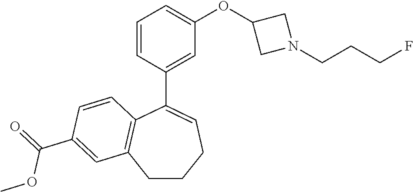 [SMILES: COC(=O)c1ccc2c(c1)CCCC=C2c1cccc(OC2CN(CCCF)C2)c1]

Step 4 of Intermediate 7 was prepared following a similar procedure to that of Step 1 of Intermediate 2 from methyl 9-(((trifluoromethyl)sulfonyl)oxy)-6,7-dihydro-5H-benzo[7]annulene-3-carboxylate (prepared according to WO2017140669) and 1-(3-fluoropropyl)-3-(3-(4,4,5,5-tetramethyl-1,3,2-dioxaborolan-2-yl)phenoxy)azetidine to give 0.57 g (94%) of methyl 9-(3-((1-(3-fluoropropyl)azetidin-3-yl)oxy)phenyl)-6,7-dihydro-5H-benzo[7]annulene-3-carboxylate.

LC/MS (m/z, MH+): 410

### Step 5: Methyl 8-bromo-9-(3-((1-(3-fluoropropyl)azetidin-3-yl)oxy)phenyl)-6,7-dihydro-5H-benzo[7]annulene-3-carboxylate

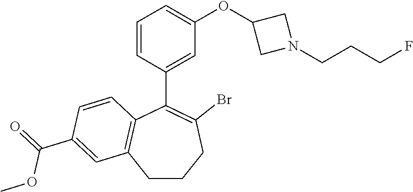 [SMILES: COC(=O)c1ccc2c(c1)CCCC(Br)=C2c1cccc(OC2CN(CCCF)C2)c1]

Step 5 of Intermediate 7 was prepared following a similar procedure to that of Step 3 of Intermediate 2 from methyl 9-(3-((1-(3-fluoropropyl)azetidin-3-yl)oxy)phenyl)-6,7-dihydro-5H-benzo[7]annulene-3-carboxylate to give 0.56 g (82%) of methyl 8-bromo-9-(3-((1-(3-fluoropropyl)azetidin-3-yl)oxy)phenyl)-6,7-dihydro-5H-benzo[7]annulene-3-carboxylate.

LC/MS (m/z, MH+): 488

### Intermediate 8: Methyl 9-(4,4,5,5-tetramethyl-1,3,2-dioxaborolan-2-yl)-6,7-dihydro-5H-benzo[7]annulene-3-carboxylate

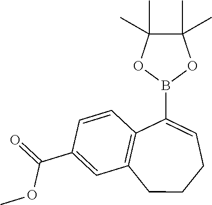

A mixture of methyl 9-(((trifluoromethyl)sulfonyl)oxy)-6,7-dihydro-5H-benzo[7]annulene-3-carboxylate (15 g, 42.82 mmol) (prepared according to WO2017140669), in toluene (150 ml), Pd(PPh₃)₂Cl₂ (1.53 g, 2.14 mmol), PPh₃ (673.87 mg, 2.57 mmol), bis(pinacolato)diboron (13.38 g, 52.67 mmol) and PhOK (8.04 g, 60.80 mmol) was heated to 75° C. for 1.5 h. The yellow suspension becomes orange then brown. After cooling to room temperature, DCM (150 mL) and water (150 mL) were added, and decantation was done by hydrophobic column. The organic phase was concentrated under reduced pressure. The residue obtained was purified by flash chromatography, eluting with a gradient of heptane/DCM: from 85/15 to 20/80 to give 10.1 g (72%) of methyl 9-(4,4,5,5-tetramethyl-1,3,2-dioxaborolan-2-yl)-6,7-dihydro-5H-benzo[7]annulene-3-carboxylate as a white solid.

LC/MS (m/z, MH+): 329

### Intermediate 9: Methyl 8-(2,4-dichlorophenyl)-9-(4,4,5,5-tetramethyl-1,3,2-dioxaborolan-2-yl)-6,7-dihydro-5H-benzo[7]annulene-3-carboxylate

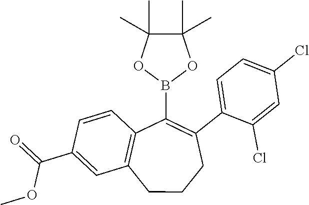 [SMILES: COC(=O)c1ccc2c(c1)CCCC(c1ccc(Cl)cc1Cl)=C2B1OC(C)(C)C(C)(C)O1]

Intermediate 9 was prepared following a similar procedure to that of Intermediate 8 from methyl 8-(2,4-dichlorophenyl)-9-(((trifluoromethyl)sulfonyl)oxy)-6,7-dihydro-5H-benzo[7]annulene-3-carboxylate (prepared according to WO2020/049153) to give 3.9 g (82%) of methyl 8-(2,4-dichlorophenyl)-9-(4,4,5,5-tetramethyl-1,3,2-dioxaborolan-2-yl)-6,7-dihydro-5H-benzo[7]annulene-3-carboxylate as a white solid.

LC/MS (m/z, MH+): 473

### Intermediate 10: Methyl 8-bromo-9-(3-((1-(3-fluoropropyl)azetidin-3-yl)methyl)phenyl)-6,7-dihydro-5H-benzo[7]annulene-3-carboxylate

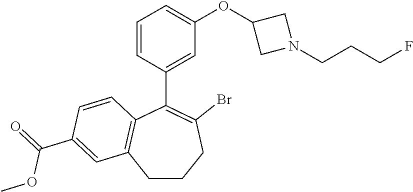 [SMILES: COC(=O)c1ccc2c(c1)CCCC(Br)=C2c1cccc(OC2CN(CCCF)C2)c1]

### Step 1: 3-(3-Bromobenzyl)-1-(3-fluoropropyl)azetidine

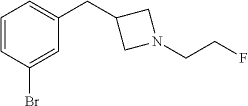 [SMILES: FCCN1CC(Cc2cccc(Br)c2)C1]

Step 1 of Intermediate 10 was prepared following a similar procedure to that of Step 3 of Intermediate 1 from 3-(3-bromobenzyl)azetidine, 2,2,2-trifluoroacetate to give 1.4 g (33%) of 3-(3-bromobenzyl)-1-(3-fluoropropyl)azetidine.

LC/MS (m/z, MH+): 286

### Step 2: Methyl 9-(3-((1-(3-fluoropropyl)azetidin-3-yl)methyl)phenyl)-6,7-dihydro-5H-benzo[7]annulene-3-carboxylate

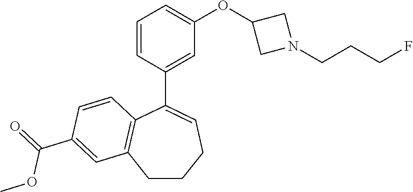 [SMILES: COC(=O)c1ccc2c(c1)CCCC=C2c1cccc(OC2CN(CCCF)C2)c1]

A mixture of methyl 9-(4,4,5,5-tetramethyl-1,3,2-dioxaborolan-2-yl)-6,7-dihydro-5H-benzo[7]annulene-3-carboxylate (0.62 g, 1.89 mmol) (Intermediate 8), 3-(3-bromobenzyl)-1-(3-fluoropropyl)azetidine (0.59 g, 1.87 mmol), Cs₂CO₃ (1.83 g, 5.61 mmol), Pd(dppf)Cl₂ complex with DCM (86 mg, 0.11 mmol), in dioxane (6 ml) and water (2.5 ml) was heated to 65° C. for 1 hour. After cooling to room temperature, addition of DCM (10 ml) and water (5 ml). After decantation on hydrophobic column, the organic phase was dried over Na₂SO₄ filtered and concentrated under reduced pressure and the residue obtained was purified by flash chromatography, eluting with DCM/MeOH: 95/05 to give 0.67 g (88%) of methyl 9-(3-((1-(3-fluoropropyl)azetidin-3-yl)methyl)phenyl)-6,7-dihydro-5H-benzo[7]annulene-3-carboxylate.

LC/MS (m/z, MH+): 408

### Step 3: Methyl 8-bromo-9-(3-((1-(3-fluoropropyl)azetidin-3-yl)methyl)phenyl)-6,7-dihydro-5H-benzo[7]annulene-3-carboxylate

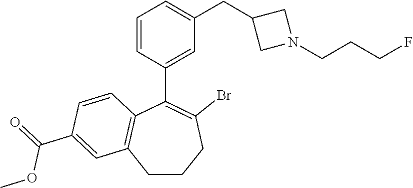 [SMILES: COC(=O)c1ccc2c(c1)CCCC(Br)=C2c1cccc(CC2CN(CCCF)C2)c1]

Step 3 of Intermediate 10 was prepared following a similar procedure to that of Step 3 of Intermediate 2 from methyl 9-(3-((1-(3-fluoropropyl)azetidin-3-yl)methyl)phenyl)-6,7-dihydro-5H-benzo[7]annulene-3-carboxylate to give 0.66 g (83%) of methyl 8-bromo-9-(3-((1-(3-fluoropropyl)azetidin-3-yl)methyl)phenyl)-6,7-dihydro-5H-benzo[7]annulene-3-carboxylate.

LC/MS (m/z, MH+): 488

### Intermediate 11: 3-[(3-Bromophenyl)methyl]-1-(3-fluoropropyl)pyrrolidine, Isomer 1

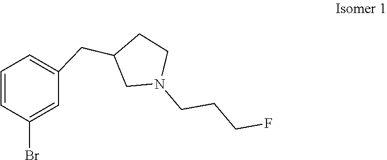 [SMILES: FCCCN1CCC(Cc2cccc(Br)c2)C1]

### Step 1: Tert-butyl 3-[(3-bromophenyl)methyl]pyrrolidine-1-carboxylate, Isomer 1 and Isomer 2

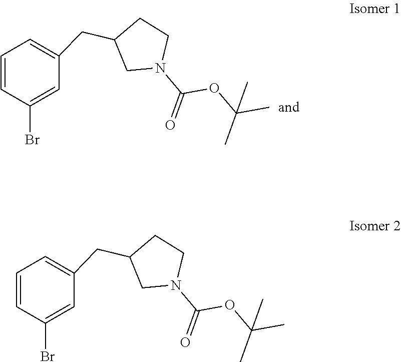 [SMILES: CC(C)(C)OC(=O)N1CCC(Cc2cccc(Br)c2)C1 ; CC(C)(C)OC(=O)N1CCC(Cc2cccc(Br)c2)C1]

To a solution of 3-[(3-bromophenyl)methyl]pyrrolidine racemic mixture (58.0 g, 242 mmol) and TEA (48.9 g, 483 mmol) in DCM (420 ml) was added DMAP (2.95 g, 24.2 mmol) and BOC₂O (55.4 g, 254 mmol) at RT. The reaction mixture was stirred at RT for 12 hours. The reaction mixture was quenched by addition of water (300 mL) and extracted with DCM (100 mL). The organic phase was washed with brine (200 mL), dried over Na₂SO₄ filtered, concentrated under reduced pressure and the residue obtained purified by flash chromatography eluting with a gradient of petroleum ether/EtOAc from 98/02 to 50/50 to give 45 g (51%) of tert-butyl 3-[(3-bromophenyl)methyl]pyrrolidine-1-carboxylate, racemic mixture.

The racemic mixture of tert-butyl 3-[(3-bromophenyl)methyl]pyrrolidine-1-carboxylate was separated by preparative SFC (column: DAICEL CHIRALCEL OJ (250×50 mm, 10 μm); supercritical CO₂ 85%/EtOH [0.1% NH₄OH]₁₅% to give 19 g of tert-butyl 3-[(3-bromophenyl)methyl]pyrrolidine-1-carboxylate Isomer 1 and 19 g of tert-butyl 3-[(3-bromophenyl)methyl]pyrrolidine-1-carboxylate Isomer 2.

LC/MS (m/z, MH+): 340

### Step 2: 3-[(3-Bromophenyl)methyl]pyrrolidine, hydrochloride Isomer 1

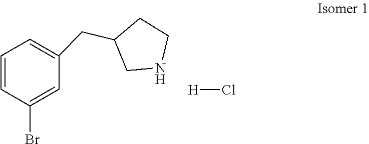

Step 2 of Intermediate 11 was prepared following a similar procedure to that of Step 2 of Intermediate 1 from tert-butyl 3-[(3-bromophenyl)methyl]pyrrolidine-1-carboxylate Isomer 1 to give 17 g (crude) of 3-[(3-bromophenyl)methyl]pyrrolidine, hydrochloride Isomer 1.

LC/MS (m/z, MH+): 240

### Step 3: 3-[(3-Bromophenyl)methyl]-1-(3-fluoropropyl)pyrrolidine, Isomer 1

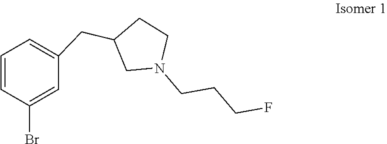 [SMILES: FCCCN1CCC(Cc2cccc(Br)c2)C1]

Step 3 of Intermediate 11 was prepared following a similar procedure to that of Step 3 of Intermediate 1 from 3-[(3-bromophenyl)methyl]pyrrolidine, hydrochloride Isomer 1 to give 10.1 g (74%) of 3-[(3-bromophenyl)methyl]-1-(3-fluoropropyl)pyrrolidine, Isomer 1.

LC/MS (m/z, MH+): 300

### Intermediate 12: Methyl 8-(2,4-dichlorophenyl)-9-(3-(((trifluoromethyl)sulfonyl)oxy)phenyl)-6,7-dihydro-5H-benzo[7]annulene-3-carboxylate

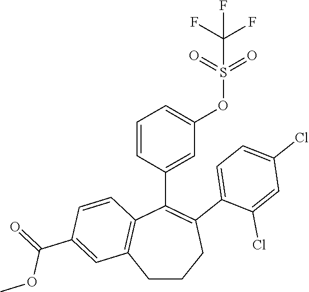 [SMILES: COC(=O)c1ccc2c(c1)CCCC(c1ccc(Cl)cc1Cl)=C2c1cccc(OS(=O)(=O)C(F)(F)F)c1]

### Step 1: Methyl 8-(2,4-dichlorophenyl)-9-(3-hydroxyphenyl)-6,7-dihydro-5H-benzo[7]annulene-3-carboxylate

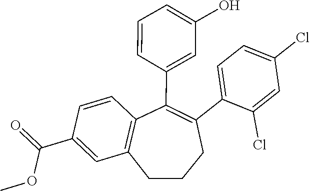 [SMILES: COC(=O)c1ccc2c(c1)CCCC(c1ccc(Cl)cc1Cl)=C2c1cccc(O)c1]

Step 1 of Intermediate 12 was prepared following a similar procedure to that of Step 1 of Intermediate 2 from 3-hydroxyphenylboronic acid and methyl 8-(2,4-dichlorophenyl)-9-(((trifluoromethyl)sulfonyl)oxy)-6,7-dihydro-5H-benzo[7]annulene-3-carboxylate (prepared according to WO2020/049153) to give 2.29 g (86%) of methyl 8-(2,4-dichlorophenyl)-9-(3-hydroxyphenyl)-6,7-dihydro-5H-benzo[7]annulene-3-carboxylate. LC/MS (m/z, MH+): 439

### Step 2: Methyl 8-(2,4-dichlorophenyl)-9-(3-(((trifluoromethyl)sulfonyl)oxy)phenyl)-6,7-dihydro-5H-benzo[7]annulene-3-carboxylate

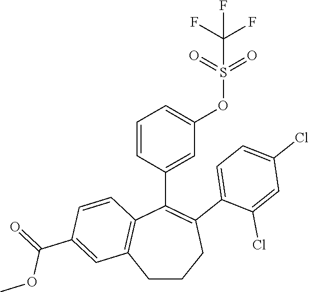 [SMILES: COC(=O)c1ccc2c(c1)CCCC(c1ccc(Cl)cc1Cl)=C2c1cccc(OS(=O)(=O)C(F)(F)F)c1]

To a mixture of methyl 8-(2,4-dichlorophenyl)-9-(3-hydroxyphenyl)-6,7-dihydro-5H-benzo[7]annulene-3-carboxylate (0.32 g, 0.73 mol) and pyridine (0.12 g, 1.46 mmol) in DCM (5 ml) cooled at 5° C. was added trifluoromethanesulfonic anhydride (0.42 g, 1.49 mmol). The reaction mixture was stirred at 5° C. for 1 hour. After addition of cooled water (5 ml) and decantation on hydrophobic column, the organic phase was dried over Na₂SO₄ filtered and concentrated under reduced pressure to give 0.48 g (crude) of methyl 8-(2,4-dichlorophenyl)-9-(3-(((trifluoromethyl)sulfonyl)oxy)phenyl)-6,7-dihydro-5H-benzo[7]annulene-3-carboxylate.

LC/MS (m/z, MH+): 571

### Intermediate 13: 8-(2,4-Dichlorophenyl)-9-(((trifluoromethyl)sulfonyl)oxy)-6,7-dihydro-5H-benzo[7]annulen-3-yl pivalate

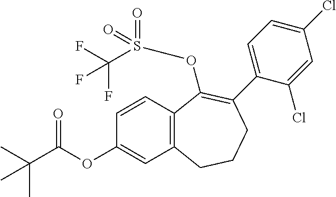 [SMILES: CC(C)(C)C(=O)Oc1ccc2c(c1)CCCC(c1ccc(Cl)cc1Cl)=C2OS(=O)(=O)C(F)(F)F]

### Step 1: 6-(2,4-Dichlorophenyl)-2-methoxy-6,7,8,9-tetrahydro-5H-benzo[7]annulen-5-one

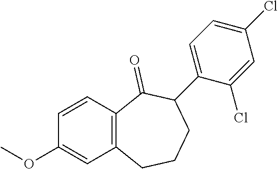 [SMILES: COc1ccc2c(c1)CCCC(c1ccc(Cl)cc1Cl)C2=O]

A mixture of 1-bromo-2,4-dichloro-benzene (890 mg, 3.94 mmol), 2-methoxy-6,7,8,9-tetrahydro-5H-benzo[7]annulen-5-one (500 m, 2.63 mmol), Cs₂CO₃ (2.03 g, 10.5 mmol), 4,5-bis(diphenylphosphino)-9,9-dimethylxanthene (0.304 g, 0.53 mmol) and tris(dibenzylideneacetone)dipalladium(0) (0.3 g, 0.53 mmol) in xylene (10 ml) was heated to reflux for 16 hours. After cooling to room temperature, water (50 ml) and Et₂O (50 ml) were added. The organic phase was dried over MgSO₄, filtered and concentrated under reduced pressure. The residue obtained was purified by flash chromatography, eluting with a gradient of cyclohexane/EtOAc from 100/00 to 95/05 to give 470 mg (42%) of 6-(2,4-dichlorophenyl)-2-methoxy-6,7,8,9-tetrahydro-5H-benzo[7]annulen-5-one.

LC/MS (m/z, MH+): 335

### Step 2: 6-(2,4-Dichlorophenyl)-2-hydroxy-6,7,8,9-tetrahydro-5H-benzo[7]annulen-5-one

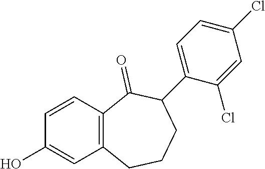 [SMILES: O=C1c2ccc(O)cc2CCCC1c1ccc(Cl)cc1Cl]

A mixture of 6-(2,4-dichlorophenyl)-2-methoxy-6,7,8,9-tetrahydro-5H-benzo[7]annulen-5-one (470 mg, 1.23 mmol) and aluminum chloride (395 mg, 2.96 mmol) in toluene (8 ml) was heated at 90° C. for 2 hours. After cooling to room temperature, water (20 ml) and EtOAc (50 ml) were added. The organic phase was dried over MgSO₄, filtered and concentrated under reduced pressure. The residue obtained was purified by flash chromatography, eluting with a gradient of cyclohexane/EtOAc from 100/00 to 80/20 to give 165 mg (42%) of 6-(2,4-dichlorophenyl)-2-hydroxy-6,7,8,9-tetrahydro-5H-benzo[7]annulen-5-one.

LC/MS (m/z, MH+): 321

### Step 3: 6-(2,4-Dichlorophenyl)-5-oxo-6,7,8,9-tetrahydro-5H-benzo[7]annulen-2-yl pivalate

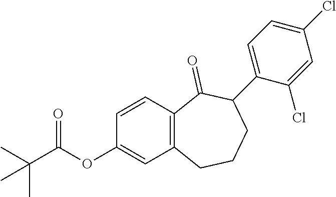 [SMILES: CC(C)(C)C(=O)Oc1ccc2c(c1)CCCC(c1ccc(Cl)cc1Cl)C2=O]

A mixture of 6-(2,4-dichlorophenyl)-2-hydroxy-6,7,8,9-tetrahydro-5H-benzo[7]annulen-5-one (163 mg, 0.51 mmol), K₂CO₃ (77 mg, 0.56 mmol) and pivaloyl chloride (67 mg, 0.56 mmol) in acetone (5 ml) was heated at 40° C. for 1 hour. After cooling to room temperature, the reaction mixture was filtered. The filtrate was concentrated under reduced pressure and the residue obtained was purified by flash chromatography with a gradient of cyclohexane/EtOAc from 100/00 to 90/10 to give 205 mg (96%) of 6-(2,4-dichlorophenyl)-5-oxo-6,7,8,9-tetrahydro-5H-benzo[7]annulen-2-yl pivalate.

LC/MS (m/z, MH+): 405

### Step 4: 8-(2,4-Dichlorophenyl)-9-(((trifluoromethyl)sulfonyl)oxy)-6,7-dihydro-5H-benzo[7]annulen-3-yl pivalate

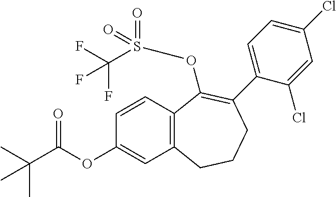 [SMILES: CC(C)(C)C(=O)Oc1ccc2c(c1)CCCC(c1ccc(Cl)cc1Cl)=C2OS(=O)(=O)C(F)(F)F]

To a solution of 6-(2,4-dichlorophenyl)-5-oxo-6,7,8,9-tetrahydro-5H-benzo[7]annulen-2-yl pivalate (197 mg, 0.48 mmol) in THF (5 ml) was added KHMDS (1 M, 0.58 ml, 0.58 mmol) at −50° C. under Ar atmosphere. The mixture was stirred for 30 min and 1,1,1-trifluoro-N-phenyl-N-(trifluoromethylsulfonyl)methanesulfonamide (173 mg, 0.49 mmol) was added to the resulting mixture. The reaction mixture was slowly warmed up to 20° C. and stirred for 90 minutes. The reaction mixture was quenched with saturated aqueous citric acid solution, then diluted with water and extracted twice with Et₂O. The combined organic layers were dried over anhydrous Na₂SO₄, filtered, and concentrated under reduced pressure. The residue was purified by flash chromatography with a gradient of cyclohexane/EtOAc from 100/00 to 90/10 to give 160 mg (61%) of 8-(2,4-dichlorophenyl)-9-(((trifluoromethyl)sulfonyl)oxy)-6,7-dihydro-5H-benzo[7]annulen-3-yl pivalate.

LC/MS (m/z, MH+): 537

### Intermediate 14: Methyl 4-(2,4-dichlorophenyl)-5-(((trifluoromethyl)sulfonyl)oxy)-2,3-dihydrobenzo[b]oxepine-8-carboxylate

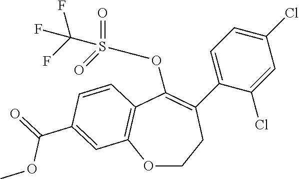 [SMILES: COC(=O)c1ccc2c(c1)OCCC(c1ccc(Cl)cc1Cl)=C2OS(=O)(=O)C(F)(F)F]

### Step 1: 5-Oxo-2,3,4,5-tetrahydrobenzo[b]oxepine-8-yl trifluoromethanesulfonate

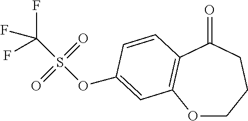 [SMILES: O=C1CCCOc2cc(OS(=O)(=O)C(F)(F)F)ccc21]

To a mixture of 8-hydroxy-3,4-dihydrobenzo[b]oxepine-5(2H)-one (4.2 g, 23.57 mmol) (prepared according to WO2018091153) and pyridine (2.82 g, 2.89 ml, 35.36 mmol) in DCM (120 ml) cooled at −20° C. was dropwise added trifluoromethanesulfonic anhydride (8.14 g, 6 ml, 28.28 mmol). The reaction mixture was stirred at 0° C. for 30 minutes. Water (50 ml) was added. The organic phase was separated and washed with an aqueous saturated solution of NaHCO₃ (50 ml). The organic phase was dried over MgSO₄, filtered, and concentrated under reduced pressure to give 7.40 g (crude) of 5-oxo-2,3,4,5-tetrahydrobenzo[b]oxepine-8-yl trifluoromethanesulfonate.

LC/MS (m/z, MH+): 311

### Step 2: Methyl 5-oxo-2,3,4,5-tetrahydrobenzo[b]oxepine-8-carboxylate

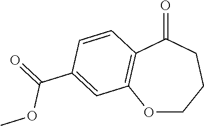 [SMILES: COC(=O)c1ccc2c(c1)OCCCC2=O]

To a solution of compound 5-oxo-2,3,4,5-tetrahydrobenzo[b]oxepine-8-yl trifluoromethanesulfonate (7.4 g, 23.85 mmol) in DMF (30 ml) and MeOH (15 ml) was added DIEA (3.15 g, 4.16 ml, 23.85 mmol) and Pd(dppf)Cl₂ complex with DCM (1.10 g, 1.43 mmol), the suspension was degassed and purged three times with CO. The mixture was stirred under CO (5 bars) at 75° C. for 2 hours. The reaction was filtered through celite. The filtrate was diluted with water (400 ml) and extracted three times with EtOAc (300 ml). The combined organic phases were dried over Na₂SO₄, filtered, concentrated under reduced pressure, and the residue obtained was purified by flash chromatography eluting with a gradient of heptane/ethyl acetate: from 85/15 to 80/20 to give 4.6 g (88%) methyl 5-oxo-2,3,4,5-tetrahydrobenzo[b]oxepine-8-carboxylate.

LC/MS (m/z, MH+): 221

### Step 3: Methyl 4-(2,4-dichlorophenyl)-5-oxo-2,3,4,5-tetrahydrobenzo[b]oxepine-8-carboxylate

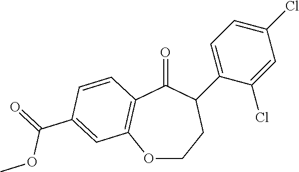 [SMILES: COC(=O)c1ccc2c(c1)OCCC(c1ccc(Cl)cc1Cl)C2=O]

Step 3 of Intermediate 14 was prepared following a similar procedure to that of step 1 of Intermediate 13 from methyl 5-oxo-2,3,4,5-tetrahydrobenzo[b]oxepine-8-carboxylate to give 1.13 g (67%) of methyl 4-(2,4-dichlorophenyl)-5-oxo-2,3,4,5-tetrahydrobenzo[b]oxepine-8-carboxylate.

LC/MS (m/z, MH+): 365

### Step 4: Methyl 4-(2,4-dichlorophenyl)-5-(((trifluoromethyl)sulfonyl)oxy)-2,3-dihydrobenzo[b]oxepine-8-carboxylate

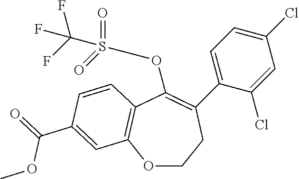 [SMILES: COC(=O)c1ccc2c(c1)OCCC(c1ccc(Cl)cc1Cl)=C2OS(=O)(=O)C(F)(F)F]

Step 4 of Intermediate 14 was prepared following a similar procedure to that of step 4 of Intermediate 13 from methyl 4-(2,4-dichlorophenyl)-5-oxo-2,3,4,5-tetrahydrobenzo[b]oxepine-8-carboxylate to give 0.48 g (31%) of methyl 4-(2,4-dichlorophenyl)-5-(((trifluoromethyl)sulfonyl)oxy)-2,3-dihydrobenzo[b]oxepine-8-carboxylate.

LC/MS (m/z, MH+): 496

### Intermediate 15: Methyl 6-(2,4-dichlorophenyl)-5-(trifluoromethylsulfonyloxy)-7,8-dihydronaphthalene-2-carboxylate

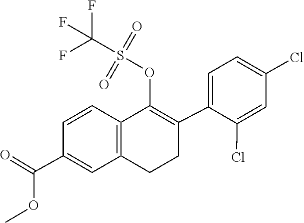 [SMILES: COC(=O)c1ccc2c(c1)CCC(c1ccc(Cl)cc1Cl)=C2OS(=O)(=O)C(F)(F)F]

### Step 1: Methyl 2-(2,4-dichlorophenyl)-1-oxo-tetralin-6-carboxylate

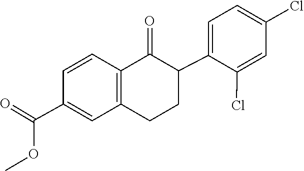 [SMILES: COC(=O)c1ccc2c(c1)CCC(c1ccc(Cl)cc1Cl)C2=O]

Step 1 of Intermediate 15 was prepared following a similar procedure to that of step 1 of Intermediate 13 from methyl 1-oxotetralin-6-carboxylate to give 459 mg (19%) of methyl 4-(2,4-dichlorophenyl)-5-oxo-2,3,4,5-tetrahydrobenzo[b]oxepine-8-carboxylate.

LC/MS (m/z, MH+): 349

### Step 2: Methyl 6-(2,4-dichlorophenyl)-5-(trifluoromethylsulfonyloxy)-7,8-dihydronaphthalene-2-carboxylate

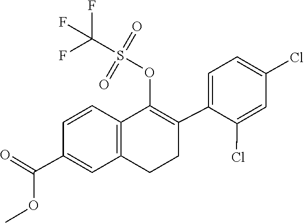 [SMILES: COC(=O)c1ccc2c(c1)CCC(c1ccc(Cl)cc1Cl)=C2OS(=O)(=O)C(F)(F)F]

Step 2 of Intermediate 15 was prepared following a similar procedure to that of step 4 of Intermediate 13 from methyl 2-(2,4-dichlorophenyl)-1-oxo-tetralin-6-carboxylate to give 0.48 g (31%) of methyl 6-(2,4-dichlorophenyl)-5-(trifluoromethylsulfonyloxy)-7,8-dihydronaphthalene-2-carboxylate.

LC/MS (m/z, MH+): 481

### Intermediate 16: (R)-3-(3-(8-bromo-6,7-dihydro-5H-benzo[7]annulen-9-yl)phenoxy)-1-(3-fluoropropyl)pyrrolidine

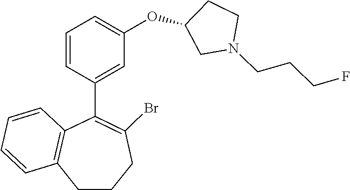 [SMILES: FCCCN1CC[C@@H](Oc2cccc(C3=C(Br)CCCc4ccccc43)c2)C1]

### Step 1: 6,7-Dihydro-5H-benzo[7]annulen-9-yl trifluoromethanesulfonate

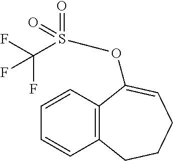 [SMILES: O=S(=O)(OC1=CCCCc2ccccc21)C(F)(F)F]

To a mixture of 6,7,8,9-tetrahydro-5H-benzo[7]annulen-5-one (1.02 g, 6.37 mmol) and pyridine (0.765 g, 0.78 ml, 9.68 mmol) in DCM (30 ml) cooled at 0° C. was dropwise added trifluoromethanesulfonic anhydride (3.61 g, 2.15 ml, 12.8 mmol). The reaction mixture was stirred at 0° C. for 30 minutes and at RT for 24 hours. Water (30 ml) was added. The organic phase was separated and washed with an aqueous saturated solution of NaHCO₃ (50 ml). The organic phase was dried over MgSO₄, filtered, and concentrated under reduced pressure to give 1.18 g (63%) of 6,7-dihydro-5H-benzo[7]annulen-9-yl trifluoromethanesulfonate LC/MS (m/z, MH+): 293

### Step 2: (R)-3-(3-(6,7-dihydro-5H-benzo[7]annulen-9-yl)phenoxy)-1-(3-fluoropropyl)pyrrolidine

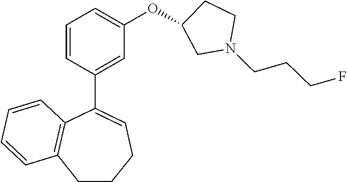 [SMILES: FCCCN1CC[C@@H](Oc2cccc(C3=CCCCc4ccccc43)c2)C1]

Step 2 of Intermediate 16 was prepared following a similar procedure to that of step 1 of Intermediate 2 from (R)-1-(3-fluoropropyl)-3-[3-(4,4,5,5-tetramethyl-1,3,2-dioxaborolan-2-yl)phenoxy]pyrrolidine (Intermediate 1) and 6,7-dihydro-5H-benzo[7]annulen-9-yl trifluoromethanesulfonate to give 0.34 g (46%) of (R)-3-(3-(6,7-dihydro-5H-benzo[7]annulen-9-yl)phenoxy)-1-(3-fluoropropyl)pyrrolidine.

LC/MS (m/z, MH+): 366

### Step 3: (R)-3-(3-(8-bromo-6,7-dihydro-5H-benzo[7]annulen-9-yl)phenoxy)-1-(3-fluoropropyl)pyrrolidine

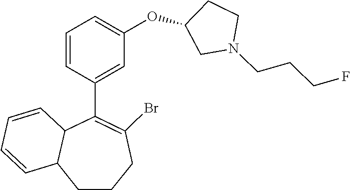 [SMILES: FCCCN1CC[C@@H](Oc2cccc(C3=C(Br)CCCC4C=CC=CC34)c2)C1]

Step 3 of Intermediate 16 was prepared following a similar procedure to that of step 3 of Intermediate 2 from (R)-3-(3-(6,7-dihydro-5H-benzo[7]annulen-9-yl)phenoxy)-1-(3-fluoropropyl)pyrrolidine to give 0.41 g (97%) of (R)-3-(3-(8-bromo-6,7-dihydro-5H-benzo[7]annulen-9-yl)phenoxy)-1-(3-fluoropropyl)pyrrolidine.

LC/MS (m/z, MH+): 444

### Intermediate 17: Methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate, Isomer 1 and Isomer 2

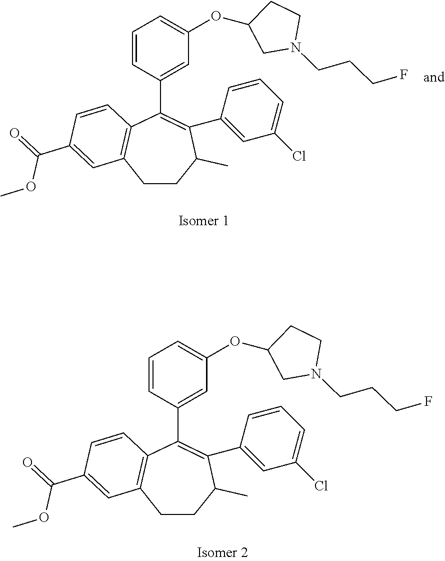 [SMILES: COC(=O)c1ccc2c(c1)CCC(C)C(c1cccc(Cl)c1)=C2c1cccc(OC2CCN(CCCF)C2)c1 ; COC(=O)c1ccc2c(c1)CCC(C)C(c1cccc(Cl)c1)=C2c1cccc(OC2CCN(CCCF)C2)c1]

### Step 1: Methyl 6-bromo-5-oxo-6,7,8,9-tetrahydro-5H-benzo[7]annulene-2-carboxylate

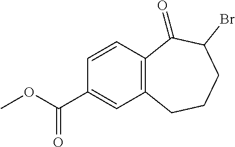 [SMILES: COC(=O)c1ccc2c(c1)CCCC(Br)C2=O]

To a mixture of methyl 5-oxo-6,7,8,9-tetrahydro-5H-benzo[7]annulene-2-carboxylate (9.42 g, 43.2 mmol) in DCM (400 mL) was portion wise added pyridinium tribromide (16.12 g, 45.4 mmol). The reaction mixture was stirred overnight at room temperature. Water (500 ml) and ether (1 L) were added. The organic phase was separated and washed twice with water, dried over MgSO₄, filtered and concentrated under reduced pressure to give 14.4 g (90%) of methyl 6-bromo-5-oxo-6,7,8,9-tetrahydro-5H-benzo[7]annulene-2-carboxylate.

LC/MS (m/z, MH+): 297

### Step 2: Methyl 5-oxo-8,9-dihydro-5H-benzo[7]annulene-2-carboxylate

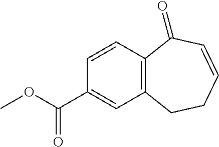 [SMILES: COC(=O)c1ccc2c(c1)CCC=CC2=O]

To a solution of methyl 6-bromo-5-oxo-6,7,8,9-tetrahydro-5H-benzo[7]annulene-2-carboxylate (10 g, 33.66 mmol) in acetonitrile (100 mL) was added DABCO (7.4 mL, 67.32 mmol). The reaction mixture was heated to 55° C. for 2.5 hours under Ar. Ether and 1N HCl were added. The organic phase was separated and washed twice with water, with brine, dried over MgSO₄, filtered and concentrated under reduced pressure. The resulting residue was then purified by flash chromatography eluting with a mixture of cyclohexane/EtOAc 85/15 to give 1.88 g (26%) of methyl 5-oxo-8,9-dihydro-5H-benzo[7]annulene-2-carboxylate.

LC/MS (m/z, MH+): 217

### Step 3: Methyl 7-methyl-5-oxo-6,7,8,9-tetrahydro-5H-benzo[7]annulene-2-carboxylate, racemic mixture

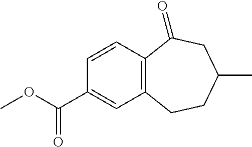

To a solution of methyl 5-oxo-8,9-dihydro-5H-benzo[7]annulene-2-carboxylate (1.88 g, 8.6 mmol) in THF (30 mL) under Ar at 0° C. was added a 0.328 M cuprate solution (35 mL, 11.5 mmol) prepared by addition of 15 mL of a 1.6 N solution of methyl lithium in ether to a suspension of 2.5 g of CuI (13 mmol) in 25 mL of ether under Ar at 0° C. The reaction mixture was stirred for 30 minutes at 0° C. Ether (200 mL) and 1N HCl (200 mL) were added. The organic phase was separated and the aqueous phase extracted with ether. The combined organic phases were washed with water, dried over MgSO₄, filtered and concentrated under reduced pressure. The resulting residue was then purified by flash chromatography eluting with a mixture of cyclohexane/EtOAc 95/5 to give 1.95 g (86%) of methyl 7-methyl-5-oxo-6,7,8,9-tetrahydro-5H-benzo[7]annulene-2-carboxylate as a racemic mixture.

LC/MS (m/z, MH+): 233

### Step 4: Methyl 7-methyl-9-(((trifluoromethyl)sulfonyl)oxy)-6,7-dihydro-5H-benzo[7]annulene-3-carboxylate, racemic mixture

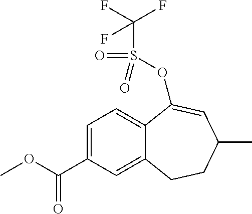

Step 4 of Intermediate 17 was prepared following a similar procedure to that of step 1 of Intermediate 16 from methyl 7-methyl-5-oxo-6,7,8,9-tetrahydro-5H-benzo[7]annulene-2-carboxylate to give 2.5 g (86%) of methyl 7-methyl-9-(((trifluoromethyl)sulfonyl)oxy)-6,7-dihydro-5H-benzo[7]annulene-3-carboxylate as a racemic mixture.

LC/MS (m/z, MH+): 365

### Step 5: Methyl 9-(3-(((R)-1-(3-fluoropropyl)pyrrolidin-3-yl)oxy)phenyl)-7-methyl-6,7-dihydro-5H-benzo[7]annulene-3-carboxylate, equimolar mixture of stereoisomers

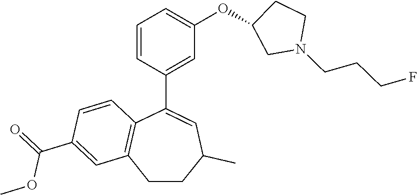 [SMILES: COC(=O)c1ccc2c(c1)CCC(C)C=C2c1cccc(O[C@@H]2CCN(CCCF)C2)c1]

Step 5 of Intermediate 17 was prepared following a similar procedure to that of step 1 of Intermediate 2 from (R)-1-(3-fluoropropyl)-3-[3-(4,4,5,5-tetramethyl-1,3,2-dioxaborolan-2-yl)phenoxy]pyrrolidine (Intermediate 1) and methyl 7-methyl-9-(((trifluoromethyl)sulfonyl)oxy)-6,7-dihydro-5H-benzo[7]annulene-3-carboxylate to give 0.306 g (48%) of methyl 9-(3-(((R)-1-(3-fluoropropyl)pyrrolidin-3-yl)oxy)phenyl)-7-methyl-6,7-dihydro-5H-benzo[7]annulene-3-carboxylate as an equimolar mixture of stereoisomers.

LC/MS (m/z, MH+): 438

### Step 6: Methyl 8-bromo-9-(3-(((R)-1-(3-fluoropropyl)pyrrolidin-3-yl)oxy)phenyl)-7-methyl-6,7-dihydro-5H-benzo[7]annulene-3-carboxylate, equimolar mixture of stereoisomers

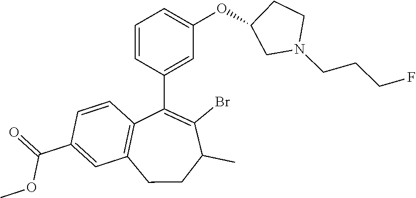 [SMILES: COC(=O)c1ccc2c(c1)CCC(C)C(Br)=C2c1cccc(O[C@@H]2CCN(CCCF)C2)c1]

Step 6 of Intermediate 17 was prepared following a similar procedure to that of step 3 of Intermediate 2 from methyl 9-(3-(((R)-1-(3-fluoropropyl)pyrrolidin-3-yl)oxy)phenyl)-7-methyl-6,7-dihydro-5H-benzo[7]annulene-3-carboxylate as an equimolar mixture of stereoisomers to give 0.48 g (31%) of (R)-3-(3-(6,7-dihydro-5H-benzo[7]annulen-9-yl)phenoxy)-1-(3-fluoropropyl)pyrrolidine to give 0.294 g (81%) of methyl 8-bromo-9-(3-(((R)-1-(3-fluoropropyl)pyrrolidin-3-yl)oxy)phenyl)-7-methyl-6,7-dihydro-5H-benzo[7]annulene-3-carboxylate, equimolar mixture of stereoisomers.

LC/MS (m/z, MH): 516

### Step 7: Methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate, Isomer 1 and Isomer 2

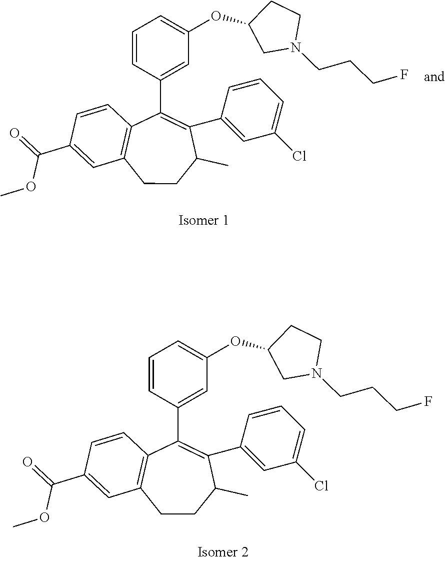 [SMILES: COC(=O)c1ccc2c(c1)CCC(C)C(c1cccc(Cl)c1)=C2c1cccc(O[C@@H]2CCN(CCCF)C2)c1 ; COC(=O)c1ccc2c(c1)CCC(C)C(c1cccc(Cl)c1)=C2c1cccc(O[C@@H]2CCN(CCCF)C2)c1]

A mixture of methyl 8-bromo-9-(3-(((R)-1-(3-fluoropropyl)pyrrolidin-3-yl)oxy)phenyl)-7-methyl-6,7-dihydro-5H-benzo[7]annulene-3-carboxylate, equimolar mixture of stereoisomers (294 mg, 0.57 mmol), 3-chlorophenylboronic acid (98 mg, 0.63 mmol), Cs₂CO₃ (464 mg, 1.42 mmol), Pd(dppf)Cl₂ complex with DCM (28 mg, 34 μmol), in dioxane (7.6 ml) and water (32 ml) was heated at 80° C. for 1 hour. After cooling to room temperature, DCM (10 ml) and water (2 ml) were added and the mixture was separated with hydrophobic column. The organic phase was dried over Na₂SO₄ filtered and concentrated under reduced pressure and the residue obtained was purified by flash chromatography, eluting with a gradient of DCM/MeOH from 100/00 to 95/05 to give 238 mg (76%) of methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate as an equimolar mixture of stereoisomers. The mixture of methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate was separated by chiral chromatography (column: I-Amylose-3 (250×30 mm, 5 μm); heptane 90%/EtOH [0.1% TEA]₁₀%) to give 88 mg of methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate Isomer 1 and 88 mg of methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate Isomer 2.

LC/MS (m/z, MH+): 548

### Intermediate 18: (R)-4-(2,4-Dichlorophenyl)-5-(3-((1-(3-fluoropropyl)pyrrolidin-3-yl)oxy)phenyl)-2,3-dihydrobenzo[b]thiepin-8-yl pivalate

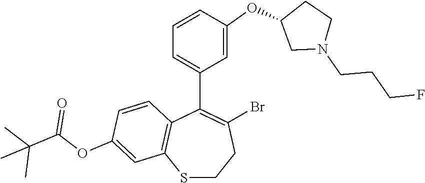 [SMILES: CC(C)(C)C(=O)Oc1ccc2c(c1)SCCC(Br)=C2c1cccc(O[C@@H]2CCN(CCCF)C2)c1]

### Step 1: (R)-5-(3-((1-(3-Fluoropropyl)pyrrolidin-3-yl)oxy)phenyl)-2,3-dihydrobenzo[b]thiepin-8-yl pivalate

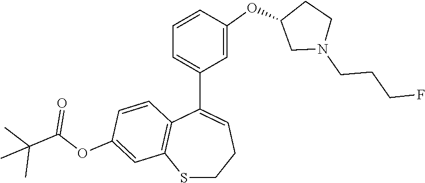 [SMILES: CC(C)(C)C(=O)Oc1ccc2c(c1)SCCC=C2c1cccc(O[C@@H]2CCN(CCCF)C2)c1]

Step 1 of Intermediate 18 was prepared following a similar procedure to that of Intermediate 2 Method 2 from 5-(((trifluoromethyl)sulfonyl)oxy)-2,3-dihydrobenzo[b]thiepin-8-yl pivalate (prepared according to WO2018091153) and (R)-1-(3-fluoropropyl)-3-[3-(4,4,5,5-tetramethyl-1,3,2-dioxaborolan-2-yl)phenoxy]pyrrolidine (Intermediate 1) to give 160 mg (77%) of (R)-5-(3-((1-(3-fluoropropyl)pyrrolidin-3-yl)oxy)phenyl)-2,3-dihydrobenzo[b]thiepin-8-yl pivalate.

LC/MS (m/z, MH+): 484

### Step 2: (R)-4-Bromo-5-(3-((1-(3-fluoropropyl)pyrrolidin-3-yl)oxy)phenyl)-2,3-dihydrobenzo[b]thiepin-8-yl pivalate

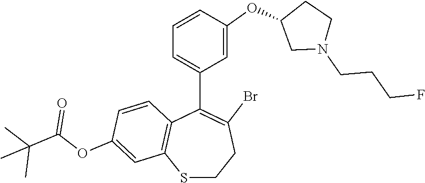 [SMILES: CC(C)(C)C(=O)Oc1ccc2c(c1)SCCC(Br)=C2c1cccc(O[C@@H]2CCN(CCCF)C2)c1]

Step 2 of Intermediate 18 was prepared following a similar procedure to that Step 3 of Intermediate 2 from (R)-5-(3-((1-(3-fluoropropyl)pyrrolidin-3-yl)oxy)phenyl)-2,3-dihydrobenzo[b]thiepin-8-yl pivalate to give 114 mg (62%) of (R)-4-bromo-5-(3-((1-(3-fluoropropyl)pyrrolidin-3-yl)oxy)phenyl)-2,3-dihydrobenzo[b]thiepin-8-yl pivalate.

LC/MS (m/z, MH+): 562

### Method A

### Example 1

### (R)-8-(2,4-Dichlorophenyl)-9-(3-((1-(3-fluoropropyl)pyrrolidin-3-yl)oxy)phenyl)-6,7-dihydro-5H-benzo[7]annulene-3-carboxylic acid

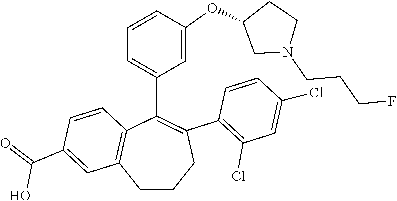 [SMILES: O=C(O)c1ccc2c(c1)CCCC(c1ccc(Cl)cc1Cl)=C2c1cccc(O[C@@H]2CCN(CCCF)C2)c1]

### Step 1: Methyl (R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylate

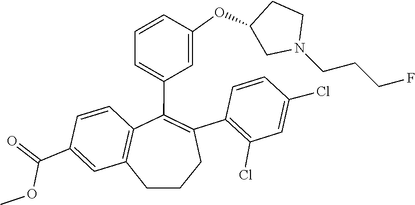

A mixture of methyl (R)-8-bromo-9-(3-((1-(3-fluoropropyl)pyrrolidin-3-yl)oxy)phenyl)-6,7-dihydro-5H-benzo[7]annulene-3-carboxylate (Intermediate 2) (100 mg, 0.20 mmol), 2,4-dichlorophenylboronic acid (43 mg, 0.22 mmol), Cs₂CO₃ (207 mg, 0.62 mmol), Pd(dppf)Cl₂ complex with DCM (11 mg, 12.7 μmol), in dioxane (1 ml) and water (0.4 ml) was heated to 80° C. under microwave irradiations for 30 minutes. After cooling to room temperature, DCM (10 ml) and water (2 ml) were added and the mixture was separated with hydrophobic column. The organic phase was dried over Na₂SO₄ filtered and concentrated under reduced pressure and the residue obtained was purified by flash chromatography, eluting with a gradient of DCM/MeOH from 100/00 to 95/05 to give 72 mg (63%) of methyl (R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylate.

LC/MS (m/z, MH+): 568

### Step 2: (R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylic acid

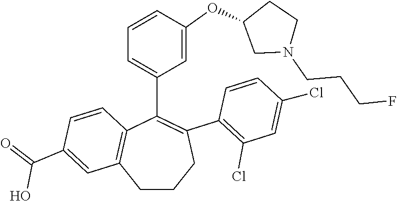 [SMILES: O=C(O)c1ccc2c(c1)CCCC(c1ccc(Cl)cc1Cl)=C2c1cccc(O[C@@H]2CCN(CCCF)C2)c1]

A mixture of methyl (R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylate (72 mg, 0.13 mmol) in MeOH (3 ml) was added NaOH 2M (185 μl, 0.37 mmol) and the reaction mixture was stirred at reflux for 3 hours. After cooling to room temperature, water (3 ml), DCM (3 ml) and HCl 1N were added to obtain a pH of 6-7. After separation with hydrophobic column, the organic phase was concentrated under reduced pressure and the residue was purified by flash chromatography, eluting with a gradient of DCM/MeOH from 100/00 to 90/10 to give 47 mg (66%) of (R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylic acid.

### Method B

### Example 10: (R)-8-(4,4-Difluorocyclohexyl)-9-(3-((1-(3-fluoropropyl)pyrrolidin-3-yl)oxy)phenyl)-6,7-dihydro-5H-benzo[7]annulene-3-carboxylic acid hydrochloride

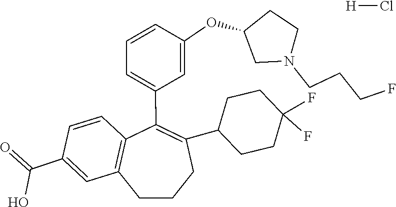 [SMILES: Cl ; O=C(O)c1ccc2c(c1)CCCC(C1CCC(F)(F)CC1)=C2c1cccc(O[C@@H]2CCN(CCCF)C2)c1]

### Step 1: Methyl (R)-8-(4,4-difluorocyclohex-1-en-1-yl)-9-(3-((1-(3-fluoropropyl)pyrrolidin-3-yl)oxy)phenyl)-6,7-dihydro-5H-benzo[7]annulene-3-carboxylate

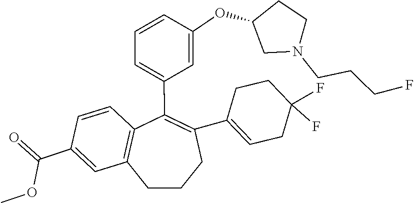 [SMILES: COC(=O)c1ccc2c(c1)CCCC(C1=CCC(F)(F)CC1)=C2c1cccc(O[C@@H]2CCN(CCCF)C2)c1]

Step 1 of Example 10 was prepared following a similar procedure to that of step 1 of Example 1 from methyl (R)-8-bromo-9-(3-((1-(3-fluoropropyl)pyrrolidin-3-yl)oxy)phenyl)-6,7-dihydro-5H-benzo[7]annulene-3-carboxylate (Intermediate 2) and (4,4-difluorocyclohex-1-en-1-yl)boronic acid to give 130 mg (61%) of methyl (R)-8-(4,4-difluorocyclohex-1-en-1-yl)-9-(3-((1-(3-fluoropropyl)pyrrolidin-3-yl)oxy)phenyl)-6,7-dihydro-5H-benzo[7]annulene-3-carboxylate.

LC/MS (m/z, MH+): 540.

### Step 2: Methyl (R)-8-(4,4-difluorocyclohexyl)-9-(3-((1-(3-fluoropropyl)pyrrolidin-3-yl)oxy)phenyl)-6,7-dihydro-5H-benzo[7]annulene-3-carboxylate

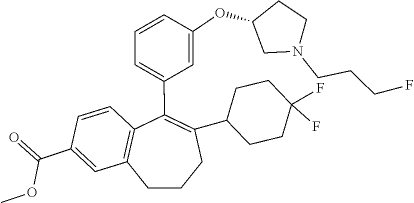 [SMILES: COC(=O)c1ccc2c(c1)CCCC(C1CCC(F)(F)CC1)=C2c1cccc(O[C@@H]2CCN(CCCF)C2)c1]

A mixture of methyl (R)-8-(4,4-difluorocyclohex-1-en-1-yl)-9-(3-((1-(3-fluoropropyl)pyrrolidin-3-yl)oxy)phenyl)-6,7-dihydro-5H-benzo[7]annulene-3-carboxylate (127 mg, 235 μmol), Pd/C 10% (12 mg, 117 μmol) in EtOH (9 ml) and EtOAc (5 ml) was hydrogenated at 65° C. and 7 bars of H₂ for 24 hours. The reaction mixture was filtered and the filtrate was concentrated under reduced pressure to give 135 mg (crude) of methyl (R)-8-(4,4-difluorocyclohexyl)-9-(3-((1-(3-fluoropropyl)pyrrolidin-3-yl)oxy)phenyl)-6,7-dihydro-5H-benzo[7]annulene-3-carboxylate which was used as such in the next step.

LC/MS (m/z, MH+): 542

### Step 3: (R)-8-(4,4-Difluorocyclohexyl)-9-(3-((1-(3-fluoropropyl)pyrrolidin-3-yl)oxy)phenyl)-6,7-dihydro-5H-benzo[7]annulene-3-carboxylic acid hydrochloride Isomer 1

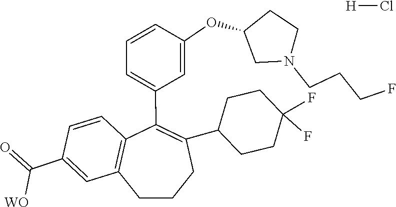

Step 3 of Example 10 was prepared following a similar procedure to that of step 2 of Example 1 from methyl (R)-8-(4,4-difluorocyclohexyl)-9-(3-((1-(3-fluoropropyl)pyrrolidin-3-yl)oxy)phenyl)-6,7-dihydro-5H-benzo[7]annulene-3-carboxylate to give 100 mg (73.9%) of (R)-8-(4,4-difluorocyclohexyl)-9-(3-((1-(3-fluoropropyl)pyrrolidin-3-yl)oxy)phenyl)-6,7-dihydro-5H-benzo[7]annulene-3-carboxylic acid hydrochloride Isomer 1.

### Method C

### Example 28: 8-(2,4-Dichlorophenyl)-9-(3-((1-(3-fluoropropyl)pyrrolidin-3-yl)methyl)phenyl)-6,7-dihydro-5H-benzo[7]annulene-3-carboxylic acid, Isomer 1

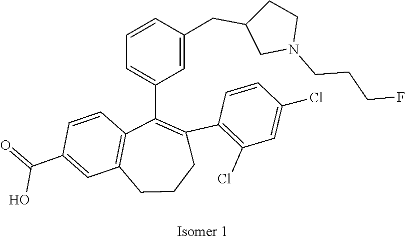 [SMILES: O=C(O)c1ccc2c(c1)CCCC(c1ccc(Cl)cc1Cl)=C2c1cccc(CC2CCN(CCCF)C2)c1]

### Step 1: Methyl 8-(2,4-dichlorophenyl)-9-(3-((1-(3-fluoropropyl)pyrrolidin-3-yl)methyl)phenyl)-6,7-dihydro-5H-benzo[7]annulene-3-carboxylate, Isomer 1

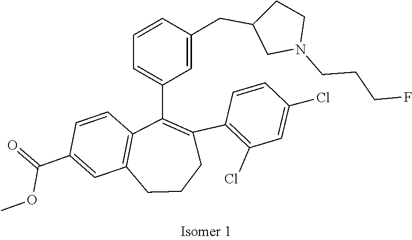 [SMILES: COC(=O)c1ccc2c(c1)CCCC(c1ccc(Cl)cc1Cl)=C2c1cccc(CC2CCN(CCCF)C2)c1]

Step 1 of Example 28 was prepared following a similar procedure to that of Step 1 of Example 1 from 3-(3-bromobenzyl)-1-(3-fluoropropyl)pyrrolidine Isomer 1 (Intermediate 11) and methy 8-(2,4-dichlorophenyl)-9-(4,4,5,5-tetramethyl-1,3,2-dioxaborolan-2-yl)-6,7-dihydro-5H-benzo[7]annulene-3-carboxylate (Intermediate 9) to give 100 mg (82%) of methyl 8-(2,4-dichlorophenyl)-9-(3-((1-(3-fluoropropyl)pyrrolidin-3-yl)ethyl)phenyl)-6,7-dihydro-5H-benzo[7]annulene-3-carboxylate, Isomer 1.

LC/MS (m/z, M+): 566

### Step 2: Methyl 8-(2,4-dichlorophenyl)-9-(3-((1-(3-fluoropropyl)pyrrolidin-3-yl)methyl)phenyl)-6,7-dihydro-5H-benzo[7]annulene-3-carboxylic acid, Isomer 1

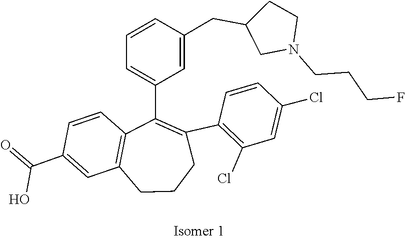 [SMILES: O=C(O)c1ccc2c(c1)CCCC(c1ccc(Cl)cc1Cl)=C2c1cccc(CC2CCN(CCCF)C2)c1]

Step 2 of Example 28 was prepared following a similar procedure to that of Step 2 of Example 1 from methyl 8-(2,4-dichlorophenyl)-9-(3-((1-(3-fluoropropyl)pyrrolidin-3-yl)methyl)phenyl)-6,7-dihydro-5H-benzo[7]annulene-3-carboxylate, Isomer 1 to give 55 mg (56%) of 8-(2,4-dichlorophenyl)-9-(3-((1-(3-fluoropropyl)pyrrolidin-3-yl)methyl)phenyl)-6,7-dihydro-5H-benzo[7]annulene-3-carboxylic acid, Isomer 1.

### Method D

### Example 32: 8-(2,4-Dichlorophenyl)-9-(3-((1-(3-fluoropropyl)azetidin-3-yl)amino)phenyl)-6,7-dihydro-5H-benzo[7]annulene-3-carboxylic acid

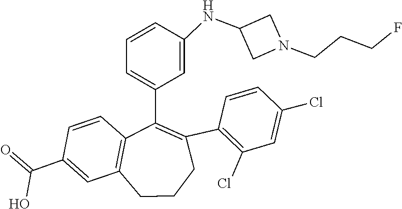 [SMILES: O=C(O)c1ccc2c(c1)CCCC(c1ccc(Cl)cc1Cl)=C2c1cccc(NC2CN(CCCF)C2)c1]

### Step 1: Tert-butyl 3-((3-(8-(2,4-dichlorophenyl)-3-(methoxycarbonyl)-6,7-dihydro-5H-benzo[7]annulen-9-yl)phenyl)amino)azetidine-1-carboxylate

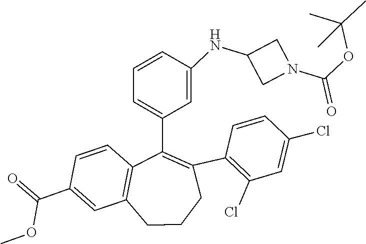 [SMILES: COC(=O)c1ccc2c(c1)CCCC(c1ccc(Cl)cc1Cl)=C2c1cccc(NC2CN(C(=O)OC(C)(C)C)C2)c1]

To a mixture of methyl 8-(2,4-dichlorophenyl)-9-(3-(((trifluoromethyl)sulfonyl)oxy)phenyl)-6,7-dihydro-5H-benzo[7]annulene-3-carboxylate (Intermediate 12) (0.42 g, 0.73 mol), tert-butyl 3-aminoazetidine-1-carboxylate (0.26 g, 1.48 mmol), Cs₂CO₃ (0.74 g, 2.22 mmol), Pd(OAc)₂ (18 mg, 0.075 mmol), 4,5-bis(diphenylphosphino)-9,9-dimethylxanthene (62 mg, 0.10 μmol) in dioxane (5 ml) was heated to 130° C. under microwave irradiations for 1 hour. After cooling to room temperature, DCM (5 ml) and water (1 ml) were added. After separation on hydrophobic column, the organic phase was dried over Na₂SO₄ filtered and concentrated under reduced pressure and the residue obtained was purified by flash chromatography eluting with a gradient of petroleum DCM/MeOH from 100/00 to 98/02 to give 0.26 g (60%) of tert-butyl 3-((3-(8-(2,4-dichlorophenyl)-3-(methoxycarbonyl)-6,7-dihydro-5H-benzo[7]annulen-9-yl)phenyl)amino)azetidine-1-carboxylate.

LC/MS (m/z, MH+): 593

### Step 2: Methyl 9-(3-(azetidin-3-ylamino)phenyl)-8-(2,4-dichlorophenyl)-6,7-dihydro-5H-benzo[7]annulene-3-carboxylate

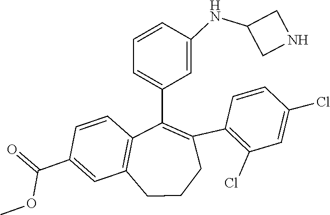 [SMILES: COC(=O)c1ccc2c(c1)CCCC(c1ccc(Cl)cc1Cl)=C2c1cccc(NC2CNC2)c1]

To a solution of tert-butyl 3-((3-(8-(2,4-dichlorophenyl-3-(bonyl)-6,7-dihydro-5H-benzo[7]annulen-9-yl)phenyl)amino)azetidine-1-carboxylate (0.26 g, 0.438 mmol) in DCM (3 ml) was added TFA (0.35 ml, 4.52 mol). The reaction mixture was stirred at RT for 2 hours. The reaction mixture was concentrated under reduced pressure. The residue obtained was purified on SCX-2 column to give 0.17 g (81%) of methyl 9-(3-(azetidin-3-ylamino)phenyl)-8-(2,4-dichlorophenyl)-6,7-dihydro-5H-benzo[7]annulene-3-carboxylate.

LC/MS (m/z, MH+): 493.

### Step 3: Methyl 8-(2,4-dichlorophenyl)-9-(3-((1-(3-fluoropropyl)azetidin-3-yl)amino)phenyl)-6,7-dihydro-5H-benzo[7]annulene-3-carboxylate

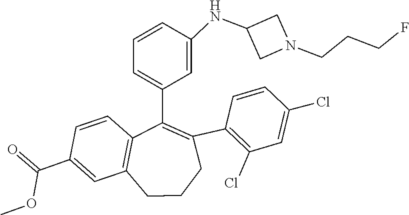 [SMILES: COC(=O)c1ccc2c(c1)CCCC(c1ccc(Cl)cc1Cl)=C2c1cccc(NC2CN(CCCF)C2)c1]

Step 3 of Example 32 was prepared following a similar procedure to that of Step 3 of Intermediate 1 from of methyl 9-(3-(azetidin-3-ylamino)phenyl)-8-(2,4-dichlorophenyl)-6,7-dihydro-5H-benzo[7]annulene-3-carboxylate to give 63 mg (33%) of methyl 8-(2,4-dichlorophenyl)-9-(3-((1-(3-fluoropropyl)azetidin-3-yl)amino)phenyl)-6,7-dihydro-5H-benzo[7]annulene-3-carboxylate.

LC/MS (m/z, MH+): 554

### Step 4: 8-(2,4-Dichlorophenyl)-9-(3-((1-(3-fluoropropyl)azetidin-3-yl)amino)phenyl)-6,7-dihydro-5H-benzo[7]annulene-3-carboxylic acid

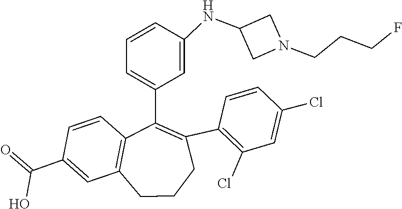 [SMILES: O=C(O)c1ccc2c(c1)CCCC(c1ccc(Cl)cc1Cl)=C2c1cccc(NC2CN(CCCF)C2)c1]

Step 4 of Example 32 was prepared following a similar procedure to that of Step 2 of Example 1 from methyl 8-(2,4-dichlorophenyl)-9-(3-((1-(3-fluoropropyl)azetidin-3-yl)amino)phenyl)-6,7-dihydro-5H-benzo[7]annulene-3-carboxylate to give 45.6 mg (74%) of 8-(2,4-dichlorophenyl)-9-(3-((1-(3-fluoropropyl)azetidin-3-yl)amino)phenyl)-6,7-dihydro-5H-benzo[7]annulene-3-carboxylic acid.

### Example 33: (R)-8-(2,4-Dichlorophenyl)-9-(3-((1-(3-fluoropropyl)pyrrolidin-3-yl)amino)phenyl)-6,7-dihydro-5H-benzo[7]annulene-3-carboxylic acid hydrochloride

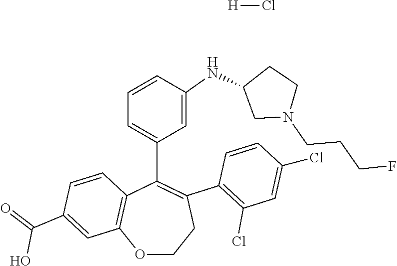 [SMILES: Cl ; O=C(O)c1ccc2c(c1)OCCC(c1ccc(Cl)cc1Cl)=C2c1cccc(N[C@@H]2CCN(CCCF)C2)c1]

### Step 1: Tert-butyl (R)-3-((3-(8-(2,4-dichlorophenyl)-3-(methoxycarbonyl)-6,7-dihydro-5H-benzo[7]annulen-9-yl)phenyl)amino)pyrrolidine-1-carboxylate

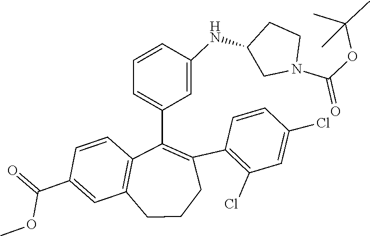 [SMILES: COC(=O)c1ccc2c(c1)CCCC(c1ccc(Cl)cc1Cl)=C2c1cccc(N[C@@H]2CCN(C(=O)OC(C)(C)C)C2)c1]

Step 1 of Example 33 was prepared following a similar procedure to that of Step 1 of Example 32 from methyl 8-(2,4-dichlorophenyl)-9-(3-(((trifluoromethyl)sulfonyl)oxy)phenyl)-6,7-dihydro-5H-benzo[7]annulene-3-carboxylate (Intermediate 12) and (R)-1-BOC-3-aminopyrrolidine to give 160 mg (13%) of tert-butyl (R)-3-((3-(8-(2,4-dichlorophenyl)-3-(methoxycarbonyl)-6,7-dihydro-5H-benzo[7]annulen-9-yl)phenyl)amino)pyrrolidine-1-carboxylate.

LC/MS (m/z, MH+): 607

### Step 2: Methyl (R)-8-(2,4-dichlorophenyl)-9-(3-(pyrrolidin-3-ylamino)phenyl)-6,7-dihydro-5H-benzo[7]annulene-3-carboxylate

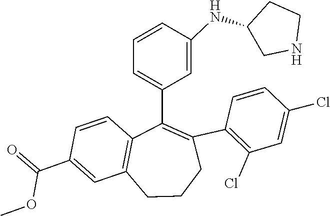 [SMILES: COC(=O)c1ccc2c(c1)CCCC(c1ccc(Cl)cc1Cl)=C2c1cccc(N[C@@H]2CCNC2)c1]

Step 2 of Example 33 was prepared following a similar procedure to that of Step 2 of Example 32 from tert-butyl (R)-3-((3-(8-(2,4-dichlorophenyl)-3-(methoxycarbonyl)-6,7-dihydro-5H-benzo[7]annulen-9-yl)phenyl)amino)pyrrolidine-1-carboxylate to give 120 mg (90%) of methyl (R)-8-(2,4-dichlorophenyl)-9-(3-(pyrrolidin-3-ylamino)phenyl)-6,7-dihydro-5H-benzo[7]annulene-3-carboxylate.

LC/MS (m/z, MH+): 507.

### Step 3: Methyl (R)-8-(2,4-dichlorophenyl)-9-(3-((1-(3-fluoropropyl)pyrrolidin-3-yl)amino)phenyl)-6,7-dihydro-5H-benzo[7]annulene-3-carboxylate

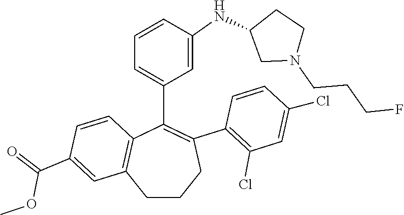 [SMILES: COC(=O)c1ccc2c(c1)CCCC(c1ccc(Cl)cc1Cl)=C2c1cccc(N[C@@H]2CCN(CCCF)C2)c1]

Step 3 of Example 33 was prepared following a similar procedure to that of Step 3 of Intermediate 1 from methyl (R)-8-(2,4-dichlorophenyl)-9-(3-(pyrrolidin-3-ylamino)phenyl)-6,7-dihydro-5H-benzo[7]annulene-3-carboxylate to give 114 mg (44%) of methyl (R)-8-(2,4-dichlorophenyl)-9-(3-((1-(3-fluoropropyl)pyrrolidin-3-yl)amino)phenyl)-6,7-dihydro-5H-benzo[7]annulene-3-carboxylate.

LC/MS (m/z, MH+): 567

### Step 4: (R)-8-(2,4-Dichlorophenyl)-9-(3-((1-(3-fluoropropyl)pyrrolidin-3-yl)amino)phenyl)-6,7-dihydro-5H-benzo[7]annulene-3-carboxylic acid hydrochloride

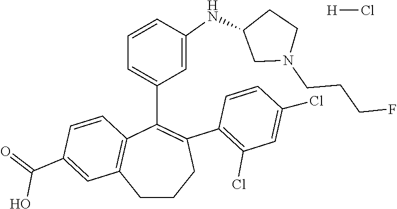

Step 4 of Example 33 was prepared following a similar procedure to that of Step 2 of Example 1 from methyl (R)-8-(2,4-dichlorophenyl)-9-(3-((1-(3-fluoropropyl)pyrrolidin-3-yl)amino)phenyl)-6,7-dihydro-5H-benzo[7]annulene-3-carboxylate to give 98 mg (83%) of (R)-8-(2,4-dichlorophenyl)-9-(3-((1-(3-fluoropropyl)pyrrolidin-3-yl)amino)phenyl)-6,7-dihydro-5H-benzo[7]annulene-3-carboxylic acid, hydrochloride.

### Method E

### Example 35: (R)-4-(2,4-Dichlorophenyl)-5-(3-((1-(3-fluoropropyl)pyrrolidin-3-yl)oxy)phenyl)-2,3-dihydrobenzo[b]oxepine-8-carboxylic acid hydrochloride

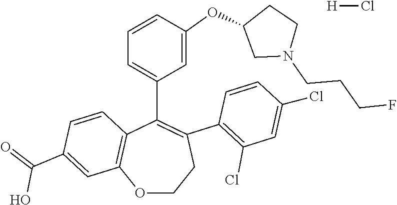

### Step 1: Methyl (R)-4-(2,4-dichlorophenyl)-5-(3-((1-(3-fluoropropyl)pyrrolidin-3-yl)oxy)phenyl)-2,3-dihydrobenzo[b]oxepine-8-carboxylate

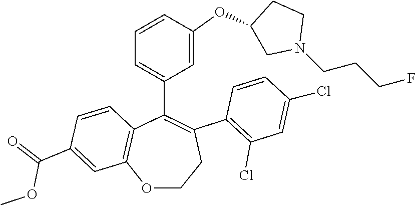

Step 1 of Example 35 was prepared following a similar procedure to that of Step 1 of Example 1 from (R)-1-(3-fluoropropyl)-3-(3-(4,4,5,5-tetramethyl-1,3,2-dioxaborolan-2-yl)phenoxy)pyrrolidine (Intermediate 1) and methyl 4-(2,4-dichlorophenyl)-5-(((trifluoromethyl)sulfonyl)oxy)-2,3-dihydrobenzo[b]oxepine-8-carboxylate (Intermediate 14) to give 57 mg (33%) of methyl (R)-4-(2,4-dichlorophenyl)-5-(3-((1-(3-fluoropropyl)pyrrolidin-3-yl)oxy)phenyl)-2,3-dihydrobenzo[b]oxepine-8-carboxylate.

LC/MS (m/z, MH+): 570

### Step 2: (R)-4-(2,4-Dichlorophenyl)-5-(3-((1-(3-fluoropropyl)pyrrolidin-3-yl)oxy)phenyl)-2,3-dihydrobenzo[b]oxepine-8-carboxylic acidhydrochloride

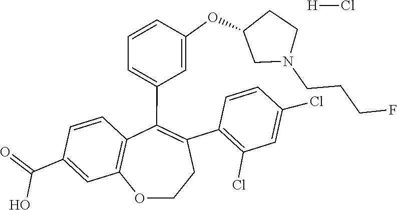

Step 2 of Example 28 was prepared following a similar procedure to that of Step 2 of Example 1 from methyl (R)-4-(2,4-dichlorophenyl)-5-(3-((1-(3-fluoropropyl)pyrrolidin-3-yl)oxy)phenyl)-2,3-dihydrobenzo[b]oxepine-8-carboxylate to give 49 mg (83%) of (R)-4-(2,4-dichlorophenyl)-5-(3-((1-(3-fluoropropyl)pyrrolidin-3-yl)oxy)phenyl)-2,3-dihydrobenzo[b]oxepine-8-carboxylic acid hydrochloride.

### Method F

### Example 34: (R)-8-(2,4-Dichlorophenyl)-9-(3-((1-(3-fluoropropyl)pyrrolidin-3-yl)oxy)phenyl)-6,7-dihydro-5H-benzo[7]annulen-3-ol

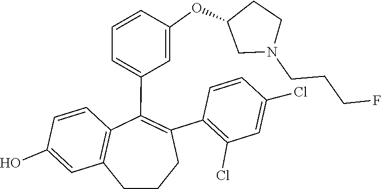

### Step 1: (R)-8-(2,4-Dichlorophenyl)-9-(3-((1-(3-fluoropropyl)pyrrolidin-3-yl)oxy)phenyl)-6,7-dihydro-5H-benzo[7]annulen-3-yl pivalate

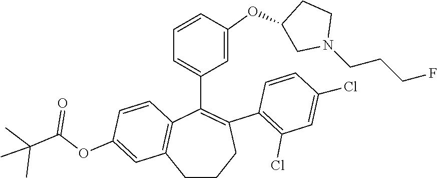

Step 1 of Example 34 was prepared following a similar procedure to that of Step 1 of Example 1 from (R)-1-(3-fluoropropyl)-3-(3-(4,4,5,5-tetramethyl-1,3,2-dioxaborolan-2-yl)phenoxy)pyrrolidine (Intermediate 1) and 8-(2,4-dichlorophenyl)-9-(((trifluoromethyl)sulfonyl)oxy)-6,7-dihydro-5H-benzo[7]annulen-3-yl pivalate (Intermediate 13) to give 105 mg (42%) of (R)-8-(2,4-dichlorophenyl)-9-(3-((1-(3-fluoropropyl)pyrrolidin-3-yl)oxy)phenyl)-6,7-dihydro-5H-benzo[7]annulen-3-yl pivalate.

LC/MS (m/z, MH+): 610

### Step 2: (R)-8-(2,4-Dichlorophenyl)-9-(3-((1-(3-fluoropropyl)pyrrolidin-3-yl)oxy)phenyl)-6,7-dihydro-5H-benzo[7]annulen-3-ol

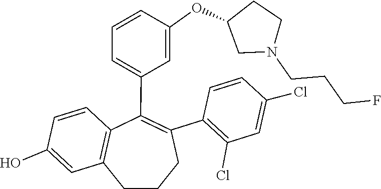

Step 2 of Example 34 was prepared following a similar procedure to that of Step 2 of Example 1 from (R)-8-(2,4-dichlorophenyl)-9-(3-((1-(3-fluoropropyl)pyrrolidin-3-yl)oxy)phenyl)-6,7-dihydro-5H-benzo[7]annulen-3-yl pivalate to give 55 mg (56%) of (R)-8-(2,4-dichlorophenyl)-9-(3-((1-(3-fluoropropyl)pyrrolidin-3-yl)oxy)phenyl)-6,7-dihydro-5H-benzo[7]annulen-3-ol.

### Example 41: (R)-4-(2,4-Dichlorophenyl)-5-(3-((1-(3-fluoropropyl)pyrrolidin-3-yl)oxy)phenyl)-2,3-dihydrobenzo[b]thiepin-8-ol

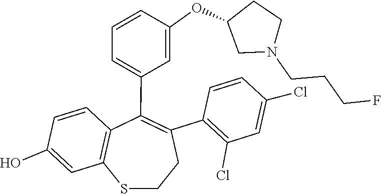

### Step 1: (R)-4-(2,4-Dichlorophenyl)-5-(3-((1-(3-fluoropropyl)pyrrolidin-3-yl)oxy)phenyl)-2,3-dihydrobenzo[b]thiepin-8-yl pivalate

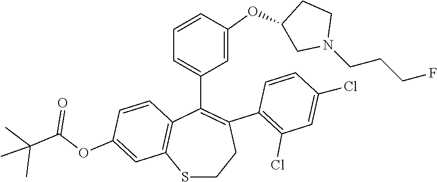

Step 1 of Example 41 was prepared following a similar procedure to that of Step 1 of Example 1 from (R)-4-bromo-5-(3-((1-(3-fluoropropyl)pyrrolidin-3-yl)oxy)phenyl)-2,3-dihydrobenzo[b]thiepin-8-yl pivalate (Intermediate 18) and 2,4-dichlorophenylboronic acid to give 67 mg (69%) of (R)-4-(2,4-dichlorophenyl)-5-(3-((1-(3-fluoropropyl)pyrrolidin-3-yl)oxy)phenyl)-2,3-dihydrobenzo[b]thiepin-8-yl pivalate.

LC/MS (m/z, M+): 628

### Step 2: (R)-4-(2,4-Dichlorophenyl)-5-(3-((1-(3-fluoropropyl)pyrrolidin-3-yl)oxy)phenyl)-2,3-dihydrobenzo[b]thiepin-8-ol

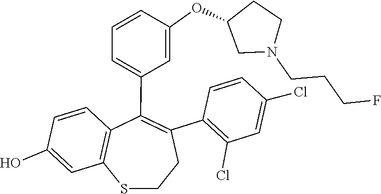

Step 2 of Example 41 was prepared following a similar procedure to that of Step 2 of Example 1 from (R)-4-(2,4-dichlorophenyl)-5-(3-((1-(3-fluoropropyl)pyrrolidin-3-yl)oxy)phenyl)-2,3-dihydrobenzo[b]thiepin-8-yl pivalate to give 23 mg (40%) of (R)-4-(2,4-dichlorophenyl)-5-(3-((1-(3-fluoropropyl)pyrrolidin-3-yl)oxy)phenyl)-2,3-dihydrobenzo[b]thiepin-8-ol.

### Method G

### Example 36: 8-(3-Chlorophenyl)-9-(3-(((R)-1-(3-fluoropropyl)pyrrolidin-3-yl)oxy)phenyl)-7-methyl-6,7-dihydro-5H-benzo[7]annulene-3-carboxylic acid, Isomer 1

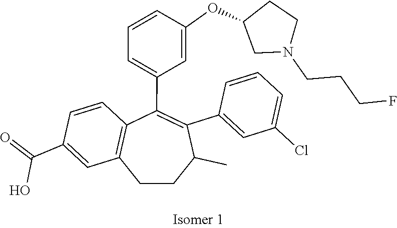

Example 36 was prepared following a similar procedure to that of Step 2 of Example 1 from methyl 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylate Isomer 1 to give 51 mg (59%) of 8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylic acid, Isomer 1.

### EXAMPLES

The compounds according to Table 1a above were subjected to pharmacological tests for determining their degradation effects on estrogen receptors.

### Test: Estrogen Receptor Degradation Activity

Said test involves measuring the in vitro degradation activity of the compounds of the Table 1a.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5 μL at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at room temperature and then washed twice with PBS. Then, 50 μL of LI-COR blocking buffer containing 0.1% Triton was added to plate for 30 minutes at room temperature. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1−(sample−fulvestrant: DMSO−fulvestrant)).

The Table 2 below indicates the estrogen receptor degradation activity results for the compounds of Table 1a tested at 0.3 μM, and demonstrates that compounds of the present invention have a significant degradation activity on estrogen receptors.

TABLE 2

| Compound No. | Degradation IC₅₀ (nM) | % Degradation At 0.3 μM |
|---|---|---|
| 1 | 0.3 | 85 |
| 1a | / | Inactive |
| 2 | 0.7 | 81 |
| 2a | / | Inactive |
| 3 | 0.4 | 84 |
| 4 | 0.6 | 87 |
| 5 | 1.1 | 91 |
| 6 | 1.8 | 89 |
| 7 | 1 | 85 |
| 8 | 0.8 | 93 |
| 9 | 0.5 | 90 |
| 10 | 1.6 | 85 |
| 11 | 6.7 | 76 |
| 12 | 1.1 | 84 |
| 13 | 14 | 57 |
| 14 | 2.2 | 77 |
| 15 | 0.9 | 80 |
| 16 | 0.7 | 97 |
| 17 | 0.8 | 87 |
| 18 | 1.2 | 78 |
| 19 | 0.6 | 82 |
| 20 | 0.3 | 88 |
| 21 | 0.5 | 85 |
| 22 | 2.8 | 76 |
| 23 | 9.7 | 84 |
| 24 | 90 | 61 |
| 25 | 3.6 | 77 |
| 26 | 0.9 | 90 |
| 27 | 1.1 | 89 |
| 28 | 3.8 | 87 |
| 29 | 0.7 | 57 |
| 30 | 0.7 | 55 |
| 31 | 38 | 58 |
| 32 | 40 | 70 |
| 33 | 30 | 79 |
| 34 | 0.6 | 74 |
| 35 | 29 | 65 |
| 36 | 0.9 | 69 |
| 37 | 79 | 67 |
| 38 | 42 | 66 |
| 39 | 41 | 78 |
| 40 | 12 | 88 |
| 41 | 17 | 75 |

It is therefore apparent that the tested compounds according to the invention have degradation activities for estrogen receptors, with IC50 less than 1 μM and with degradation levels greater than 50%. The compounds of formula (I) can therefore be used for preparing medicaments, especially medicaments which are degraders of estrogen receptors.

Further, it is apparent that the comparative compound 1a and 2a, with —O-pyrrolidine side chains in the meta position and which are both (S) stereoisomers, are inactive.

Compound 1a can be compared to compound No 51 of WO 2017/140669 A1 and compound No 43d of Youssef El-Ahmad et al. (J. Med. Chem., 2020, 63, 512-528). Compound 2a can be compared to compound No 217 of WO 2017/140669 A1. The three compounds of prior art have O-pyrrolidine side chains in the para position, are (S) stereoisomers, and have degradation activities for estrogen receptors.

Therefore, the data show that the change of the lateral chain led to a loss of biological activity, when merely moving the lateral chain from the para to the meta position.

Surprisingly, activity was retained only after the stereochemistry was changed from (S) to (R).

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:

| | | |
|---|---|---|
| Compound of formula (I) | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 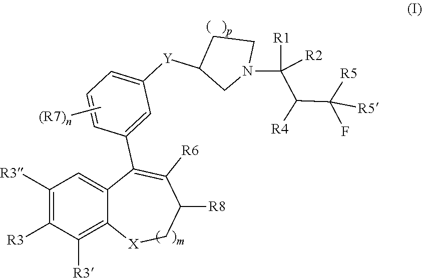
wherein:
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroatoms being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH₂—, —O— or —NH—;
R7 independently represents a (C₁-C₃)alkyl group, a halogen atom, a cyano group, or a (C₁-C₃)fluoroalkyl group;
R8 represents a hydrogen atom, or a (C₁-C₃)alkyl group or a cyclopropyl;
n is 0, 1 or 2;
m is 0 or 1; and
p is 0 or 1;
with the proviso that when Y represents —O— or —NH—, and p is 1, then the asymmetric carbon of the pyrrolidine linked to Y, is of (R) configuration.

2. The compound of formula (I) according to claim 1, or a pharmaceutically acceptable salt thereof, characterized in that R1 and R2 are a hydrogen atom.

3. The compound of formula (I) according to claim 1, or a pharmaceutically acceptable salt thereof, characterized in that R3 is a —COOH group.

4. The compound of formula (I) according to claim 1, or a pharmaceutically acceptable salt thereof, characterized in that R3′ and R3″ represent a hydrogen atom.

5. The compound of formula (I) according to claim 1, or a pharmaceutically acceptable salt thereof, characterized in that R4 represent a hydrogen atom.

6. The compound of formula (I) according to claim 1, or a pharmaceutically acceptable salt thereof, characterized in that X represents —O— or —CH₂—.

7. The compound of formula (I) according to claim 1, or a pharmaceutically acceptable salt thereof, characterized in that R5 and R5′ represent a hydrogen atom.

8. The compound of formula (I) according to claim 1, or a pharmaceutically acceptable salt thereof, characterized in that R6 represents a phenyl group, said phenyl group being optionally substituted with 1 or 2 substituents independently selected from a chlorine atom, a fluorine atom, and a methyl group.

9. The compound of formula (I) according to claim 1, or a pharmaceutically acceptable salt thereof, characterized in that R6 represents a cyclohexyl group, said cyclohexyl group being substituted with 1 or 2 substituents independently selected from a chlorine atom and a methyl group.

10. The compound of formula (I) according to claim 1, or a pharmaceutically acceptable salt thereof, characterized in that R6 represents a cyclopentyl group.

11. The compound of formula (I) according to claim 1, or a pharmaceutically acceptable salt thereof, characterized in that R7 represents a methyl group or a fluorine atom and n is 0 or 1.

12. The compound of formula (I) according to claim 1, or a pharmaceutically acceptable salt thereof, characterized in that R8 represents a hydrogen atom or a methyl group.

13. The compound of formula (I) according to claim 1, or a pharmaceutically acceptable salt thereof, wherein Y represents —O—.

14. The compound of formula (I) according to claim 1, or a pharmaceutically acceptable salt thereof, wherein m is 1.

15. The compound of formula (I) according to claim 1, or a pharmaceutically acceptable salt thereof, wherein p is 1.

16. The compound of formula (I) according to claim 1, or a pharmaceutically acceptable salt thereof, characterized in that said compound is selected from the following compounds:
(R)-8-(2,4-dichlorophenyl)-9-(3-((1-(3-fluoropropyl)pyrrolidin-3-yl)oxy)phenyl)-6,7-dihydro-5H-benzo[7]annulene-3-carboxylic acid, (1)
(R)-9-(3-((1-(3-fluoropropyl)pyrrolidin-3-yl)oxy)phenyl)-8-phenyl-6,7-dihydro-5H-benzo[7]annulene-3-carboxylic acid, (2)
(R)-8-(2-chlorophenyl)-9-(3-((1-(3-fluoropropyl)pyrrolidin-3-yl)oxy)phenyl)-6,7-dihydro-5H-benzo[7]annulene-3-carboxylic acid, (3)
(R)-8-(2-chloro-4-methylphenyl)-9-(3-((1-(3-fluoropropyl)pyrrolidin-3-yl)oxy)phenyl)-6,7-dihydro-5H-benzo[7]annulene-3-carboxylic acid, (4)
(R)-8-(2-chloro-4-fluorophenyl)-9-(3-((1-(3-fluoropropyl)pyrrolidin-3-yl)oxy)phenyl)-6,7-dihydro-5H-benzo[7]annulene-3-carboxylic acid, (5)
(R)-8-(4-chloro-2-fluorophenyl)-9-(3-((1-(3-fluoropropyl)pyrrolidin-3-yl)oxy)phenyl)-6,7-dihydro-5H-benzo[7]annulene-3-carboxylic acid, (6)
(R)-8-(2-fluoro-4-methylphenyl)-9-(3-((1-(3-fluoropropyl)pyrrolidin-3-yl)oxy)phenyl)-6,7-dihydro-5H-benzo[7]annulene-3-carboxylic acid, (7)
(R)-8-(2,4-dimethylphenyl)-9-(3-((1-(3-fluoropropyl)pyrrolidin-3-yl)oxy)phenyl)-6,7-dihydro-5H-benzo[7]annulene-3-carboxylic acid, (8)
(R)-8-(2-chloro-3-fluorophenyl)-9-(3-((1-(3-fluoropropyl)pyrrolidin-3-yl)oxy)phenyl)-6,7-dihydro-5H-benzo[7]annulene-3-carboxylic acid, (9)
(R)-8-(4,4-difluorocyclohexyl)-9-(3-((1-(3-fluoropropyl)pyrrolidin-3-yl)oxy)phenyl)-6,7-dihydro-5H-benzo[7]annulene-3-carboxylic acid hydrochloride, (10)
(R)-9-(3-((1-(3-fluoropropyl)pyrrolidin-3-yl)oxy)phenyl)-8-(4-methylcyclohexyl)-6,7-dihydro-5H-benzo[7]annulene-3-carboxylic acid, Isomer 1, (11)
(R)-9-(3-((1-(3-fluoropropyl)pyrrolidin-3-yl)oxy)phenyl)-8-(4-methylcyclohexyl)-6,7-dihydro-5H-benzo[7]annulene-3-carboxylic acid, Isomer 2, (12)
(R)-8-(4,4-dimethylcyclohexyl)-9-(3-((1-(3-fluoropropyl)pyrrolidin-3-yl)oxy)phenyl)-6,7-dihydro-5H-benzo[7]annulene-3-carboxylic acid, hydrochloride, (13)
(R)-8-cyclopentyl-9-(3-((1-(3-fluoropropyl)pyrrolidin-3-yl)oxy)phenyl)-6,7-dihydro-5H-benzo[7]annulene-3-carboxylic acid, (14)
(R)-9-(3-((1-(3-fluoropropyl)pyrrolidin-3-yl)oxy)-2-methylphenyl)-8-phenyl-6,7-dihydro-5H-benzo[7]annulene-3-carboxylic acid, (15)
(R)-8-(2-chlorophenyl)-9-(3-((1-(3-fluoropropyl)pyrrolidin-3-yl)oxy)-2-methylphenyl)-6,7-dihydro-5H-benzo[7]annulene-3-carboxylic acid, (16)
(R)-8-(2,4-dichlorophenyl)-9-(3-((1-(3-fluoropropyl)pyrrolidin-3-yl)oxy)-2-methylphenyl)-6,7-dihydro-5H-benzo[7]annulene-3-carboxylic acid, (17)
(R)-8-(4-fluoro-2-methylphenyl)-9-(3-((1-(3-fluoropropyl)pyrrolidin-3-yl)oxy)-2-methylphenyl)-6,7-dihydro-5H-benzo[7]annulene-3-carboxylic acid, (18)
(R)-9-(2-fluoro-3-((1-(3-fluoropropyl)pyrrolidin-3-yl)oxy)phenyl)-8-phenyl-6,7-dihydro-5H-benzo[7]annulene-3-carboxylic acid, hydrochloride, (19)
(R)-8-(2-chlorophenyl)-9-(2-fluoro-3-((1-(3-fluoropropyl)pyrrolidin-3-yl)oxy)phenyl)-6,7-dihydro-5H-benzo[7]annulene-3-carboxylic acid, hydrochloride, (20)
(R)-8-(2,4-dichlorophenyl)-9-(2-fluoro-3-((1-(3-fluoropropyl)pyrrolidin-3-yl)oxy)phenyl)-6,7-dihydro-5H-benzo[7]annulene-3-carboxylic acid, hydrochloride, (21)
(R)-9-(5-((1-(3-fluoropropyl)pyrrolidin-3-yl)oxy)-2-methylphenyl)-8-phenyl-6,7-dihydro-5H-benzo[7]annulene-3-carboxylic acid, (22)
(R)-8-(2-chlorophenyl)-9-(5-((1-(3-fluoropropyl)pyrrolidin-3-yl)oxy)-2-methylphenyl)-6,7-dihydro-5H-benzo[7]annulene-3-carboxylic acid, (23)
(R)-8-(2-chlorophenyl)-9-(5-((1-(3-fluoropropyl)pyrrolidin-3-yl)oxy)-2-methylphenyl)-6,7-dihydro-5H-benzo[7]annulene-3-carboxylic acid, (24)
(R)-9-(2-fluoro-5-((1-(3-fluoropropyl)pyrrolidin-3-yl)oxy)phenyl)-8-phenyl-6,7-dihydro-5H-benzo[7]annulene-3-carboxylic acid, hydrochloride, (25)
(R)-8-(2-chlorophenyl)-9-(2-fluoro-5-((1-(3-fluoropropyl)pyrrolidin-3-yl)oxy)phenyl)-6,7-dihydro-5H-benzo[7]annulene-3-carboxylic acid, hydrochloride, (26)
(R)-8-(2,4-dichlorophenyl)-9-(2-fluoro-5-((1-(3-fluoropropyl)pyrrolidin-3-yl)oxy)phenyl)-6,7-dihydro-5H-benzo[7]annulene-3-carboxylic acid hydrochloride, (27)
8-(2,4-dichlorophenyl)-9-(3-((1-(3-fluoropropyl)pyrrolidin-3-yl)methyl)phenyl)-6,7-dihydro-5H-benzo[7]annulene-3-carboxylic acid, Isomer 1, (28)
8-(2-chlorophenyl)-9-(3-((1-(3-fluoropropyl)azetidin-3-yl)oxy)phenyl)-6,7-dihydro-5H-benzo[7]annulene-3-carboxylic acid hydrochloride, (29)
8-(2,4-dichlorophenyl)-9-(3-((1-(3-fluoropropyl)azetidin-3-yl)oxy)phenyl)-6,7-dihydro-5H-benzo[7]annulene-3-carboxylic acid hydrochloride, (30)
8-(2,4-dichlorophenyl)-9-(3-((1-(3-fluoropropyl)azetidin-3-yl)methyl)phenyl)-6,7-dihydro-5H-benzo[7]annulene-3-carboxylic acid, (31)
8-(2,4-dichlorophenyl)-9-(3-((1-(3-fluoropropyl)azetidin-3-yl)amino)phenyl)-6,7-dihydro-5H-benzo[7]annulene-3-carboxylic acid, (32)
(R)-8-(2,4-dichlorophenyl)-9-(3-((1-(3-fluoropropyl)pyrrolidin-3-yl)amino)phenyl)-6,7-dihydro-5H-benzo[7]annulene-3-carboxylic acid hydrochloride, (33)
(R)-8-(2,4-dichlorophenyl)-9-(3-((1-(3-fluoropropyl)pyrrolidin-3-yl)oxy)phenyl)-6,7-dihydro-5H-benzo[7]annulen-3-ol, (34)
(R)-4-(2,4-dichlorophenyl)-5-(3-((1-(3-fluoropropyl)pyrrolidin-3-yl)oxy)phenyl)-2,3-dihydrobenzo[b]oxepine-8-carboxylic acid hydrochloride, (35)
8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylic acid, Isomer 1, (36)
8-(3-chlorophenyl)-9-(3-(((R)-1-(3-fluoropropyl)pyrrolidin-3-yl)oxy)phenyl)-7-methyl-6,7-dihydro-5H-benzo[7]annulene-3-carboxylic acid, Isomer 2, (37)
(R)-3-(3-(8-(2,4-dichlorophenyl)-6,7-dihydro-5H-benzo[7]annulen-9-yl)phenoxy)-1-(3-fluoropropyl)pyrrolidine, (38)
(R)-3-(3-(8-(2chlorophenyl)-6,7-dihydro-5H-benzo[7]annulen-9-yl)phenoxy)-1-(3-fluoropropyl)pyrrolidine, (39)
(R)-6-(2,4-dichlorophenyl)-5-(3-((1-(3-fluoropropyl)pyrrolidin-3-yl)oxy)phenyl)-7,8-dihydronaphthalene-2-carboxylic acid hydrochloride, (40), and
(R)-4-(2,4-dichlorophenyl)-5-(3-((1-(3-fluoropropyl)pyrrolidin-3-yl)oxy)phenyl)-2,3-dihydrobenzo[b]thiepin-8-ol, (41).

17. A process for preparing a compound of formula (I) as defined in claim 1, or a pharmaceutically acceptable salt thereof, wherein a compound of formula 1G: 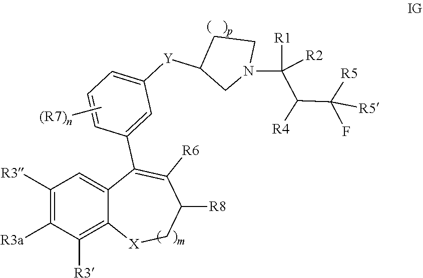
wherein R1, R2, R3′, R3″, R4, R5, R5′, R6, R7, R8, m, n, p, X and Y are as defined in claim 1, and R3a is a hydrogen atom or a carboxylic ester or protected —OH, is converted to compound of formula (I), in presence of a source of hydroxide ions in solution in methanol, said step being optionally preceded by a step for obtaining compound 1G, wherein a compound of formula 1F: 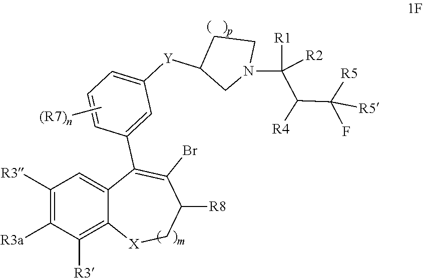
wherein R1, R2, R3′, R3″, R4, R5, R5′, R7, R8, m, n, p, X and Y are as defined in claim 1, and R3a is as defined above,
is subjected to a Suzuki coupling with a boronic reagent R6B(OR′)₂, wherein —B(OR′)₂ is a boronic acid or a pinacolate ester and R6 is as defined in claim 1.

18. A process for preparing a compound of formula (I) as defined in claim 1, or a pharmaceutically acceptable salt thereof, wherein a compound of formula 1Fa: 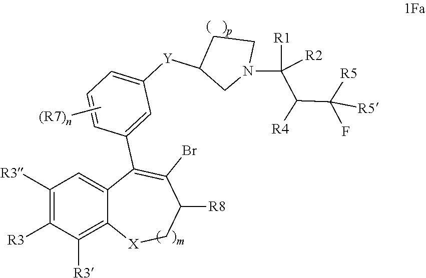
wherein R1, R2, R3, R3′, R3″, R4, R5, R5′, R7, R8, m, n, p, X and Y are as defined in claim 1, is submitted to a Suzuki coupling with a boronic reagent R6B(OR′)₂, wherein —B(OR′)₂ is a boronic acid or a pinacolate ester and R6 is defined as in claim 1, said step being optionally preceded by a step for obtaining compound 1Fa, wherein a compound of formula 1F: 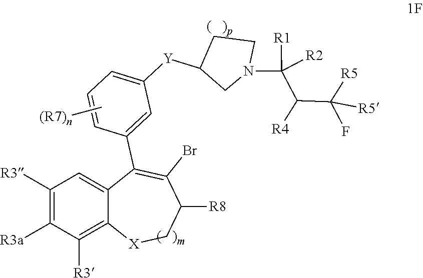
wherein R1, R2, R3′, R3″, R4, R5, R5′, R7, R8, m, n, p, X and Y are as defined in claim 1, and R3a is a hydrogen atom or a carboxylic ester or protected —OH,
is converted to a compound 1Fa in the presence of a source of hydroxide ions in solution in methanol.

19. An intermediate compound selected from a compound of formula 1E, 1F, 1G and 1Fa, or a pharmaceutically acceptable salt thereof: 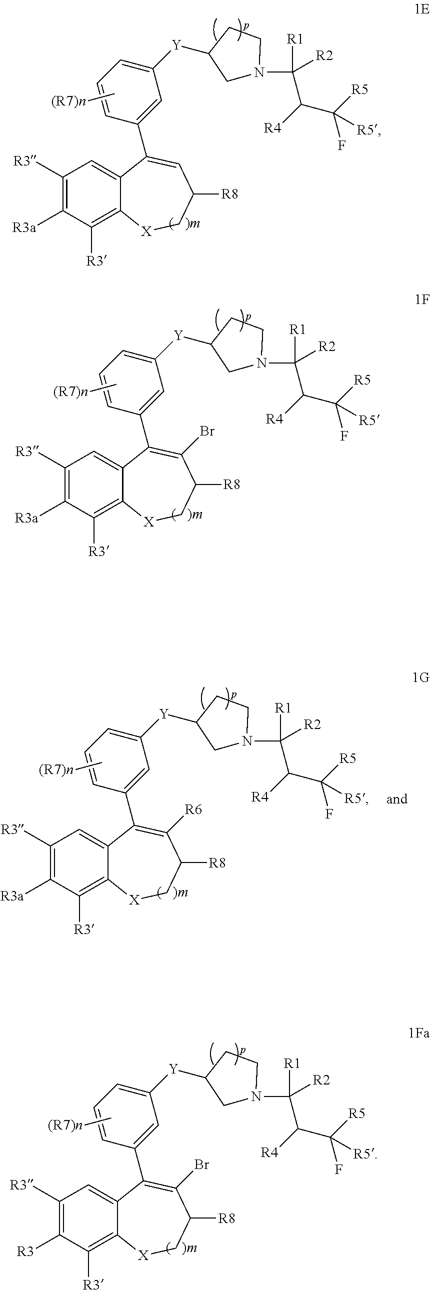
wherein
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroatoms being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group, and a phenyl group, said (C₃-C₆)cycloalkyl or phenyl groups being optionally substituted with one or two halogen atom(s) or (C₁-C₃)alkyl group(s):
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH₂—, —O— or —NH—;
R7 independently represents a (C₁-C₃)alkyl group, a halogen atom, a cyano group, or a (C₁-C₃)fluoroalkyl group;
R8 represents a hydrogen atom, or a (C₁-C₃)alkyl group or a cyclopropyl;
n is 0, 1 or 2;
m is 0 or 1; and
p is 0 or 1;
with the proviso that when Y represents —O— or —NH—, and p is 1, then the asymmetric carbon of the pyrrolidine linked to Y, is of (R) configuration; and wherein
R3a is a hydrogen atom or a carboxylic ester protected —OH.

20. An intermediate compound of formula 1D′, or a pharmaceutically acceptable salt thereof: 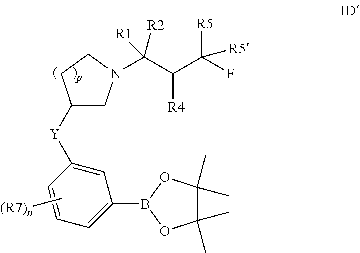
wherein
R1 and R2 independently represent a hydrogen atom or a deuterium atom;
R4 represents a hydrogen atom or a fluorine atom;
R5 and R5′ independently represent a hydrogen atom or a fluorine atom;
Y represents —CH₂—, —O— or —NH—;
R7 independently represents a (C₁-C₃)alkyl group, a halogen atom, a cyano group, or a (C₁-C₃)fluoroalkyl group;
n is 0, 1 or 2; and
p is 0 or 1;
with the proviso that when Y represents —O— or —NH—, and p is 1, then the asymmetric carbon of the pyrrolidine linked to Y, is of (R) configuration.

21. (canceled)

22. A pharmaceutical composition, characterized in that it comprises a compound of formula (I) according to claim 1, or a pharmaceutically acceptable salt thereof, and at least one pharmaceutically acceptable excipient.

23. A method for inhibiting and degrading estrogen receptors, which method comprises administering to a subject in need thereof a therapeutically effective amount of a compound of formula (I) according to claim 1, or a pharmaceutically acceptable salt thereof.

24. A method of treating ovulatory dysfunction, cancer, endometriosis, benign prostatic hypertrophy or inflammation, which method comprises administering to a subject in need thereof a therapeutically effective amount of a compound of formula (I) according to claim 1, or a pharmaceutically acceptable salt thereof.

25. The method of claim 24, which method comprises the treatment of cancer.
